(19) United States
(12) Patent Application Publication
(10) Pub. No.: US 2023/0285261 A1
(43) Pub. Date: Sep. 14, 2023

(54) FRAGRANCE COMPOSITION COMPRISING A FRAGRANCE COMPONET AND A NON-ODOROUS FRAGRANCE MODULATOR

(71) Applicant: Coty Inc., New York, NY (US)

(72) Inventors: Mariano Agustin Rodriguez-Fraticelli, Versoix (CH); Jose Maria Velazquez Mendoza, Nyon (CH); Marta Alexandra de Sousa Oliveira Maia, Versoix (CH)

(21) Appl. No.: 18/000,678

(22) PCT Filed: Jun. 3, 2021
(86) PCT No.: PCT/US2021/035668

§ 371 Date: Dec. 4, 2022

Related U.S. Application Data
(60) Provisional application No. 63/035,083, filed on Jun. 5, 2020.

(51) Int. Cl. A61K 8/60; A61K 8/11; A61Q 13/00

(52) U.S. Cl. CPC A61K 8/608; A61K 8/11; A61Q 13/00; A61K 2800/56

(57) ABSTRACT

Various embodiments according to the present disclosure provide a coating composition. The coating composition includes a carrier. The coating composition further includes a substantially non-odorous fragrance modulator present in the amount of from about 0.1 wt % to about 50 wt %, relative to the total weight of the composition.

### CROSS-REFERENCE TO RELATED APPLICATIONS

This application claims the benefit of priority to U.S. Provisional Patent Application Ser. No. 63/035,083 entitled “COATING COMPOSITIONS,” filed Jun. 5, 2020, the disclosure of which is incorporated herein in its entirety by reference.

### BACKGROUND

Fragrances can include fragrance components that can be classified, in part, by their volatility. Accordingly, these fragrance components may be referred to as a high-, middle-, or low-volatility fragrance components. Different fragrances may be dominated by any one or more of these components such that the fragrance may be associated with different perceptions by a user.

### SUMMARY OF THE DISCLOSURE

Various embodiments according to the present disclosure provide a coating composition. The coating composition includes a carrier. The coating composition further includes a substantially non-odorous fragrance modulator present in the amount of from about 0.1 wt % to about 20 wt %, relative to the total weight of the composition.

Various embodiments according to the present disclosure provide a multi-component fragrance composition. The multi-component fragrance composition includes a coating composition. The coating composition includes a carrier. The coating composition further includes a substantially non-odorous fragrance modulator present in the amount of from about 0.1 wt % to about 20 wt %, relative to the total weight of the composition. The multi-component fragrance composition further includes a fragrance composition in contact with the coating composition.

Various embodiments according to the present disclosure further provide a method of using a coating composition. The coating composition includes a carrier. The coating composition further includes a substantially non-odorous fragrance modulator present in the amount of from about 0.1 wt % to about 20 wt %, relative to the total weight of the composition. The method includes contacting a fragrance composition with the coating composition.

### BRIEF DESCRIPTION OF THE FIGURES

The drawings illustrate generally, by way of example, but not by way of limitation, various embodiments discussed in the present document.

FIG. 1 is a set of graphs identifying the most frequent comments by experts comparing a sample with coating vs. control (without coating) at t=3 h, in accordance with various embodiments.

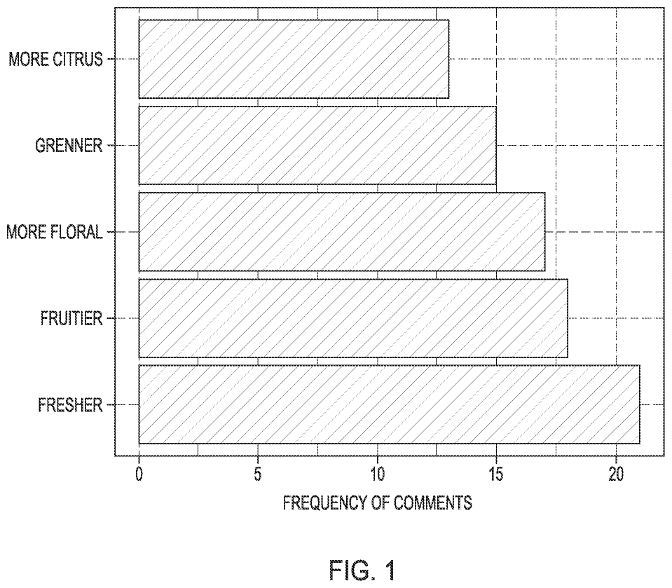

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

As used herein, articles such as “a” and “an” when used in a claim, are understood to mean one or more of what is claimed or described.

As used herein, the terms “include”, “includes” and “including” are meant to be non-limiting.

As used herein, the term “body splash” means a body care formulation that is applied to the body. Typically, the body splash is applied to the body after bathing and provides a subtle hint of scent to the body. Body splashes are commonly used by consumers who prefer less strong fragrance compositions. A body splash may comprise an ethanol-free composition according to the present invention which comprises from 0.2-8 wt %, relative to the total weight of the composition, of a fragrance component. The body splash may further comprise alkyl polyglucosides as non-ionic surfactants.

As used herein, the term “body spray” means a formulation comprising fragrance materials intended to be applied to the body to prevent or mask body odor caused by the bacterial breakdown of perspiration on the body (e.g., armpits, feet, and other areas of the body). The body spray may also provide a fragrance expression to the consumers. Typically, body spray compositions are applied as an aerosol spray in an effective amount on the skin of a consumer.

As used herein, the term “composition” includes a fine fragrance composition intended for application to a body surface, such as for example, skin or hair, e.g., to impart a pleasant odor thereto, or cover a malodour thereof. They are generally in the form of perfume concentrates, perfumes, eau de parfums, eau de toilettes, aftershaves, or colognes. The fine fragrance compositions may be an ethanol-based composition. The term “composition” may also include a cosmetic composition, which comprises a fragrance material for the purposes of delivering a pleasant smell to drive consumer acceptance of the cosmetic composition. The term “composition” may also include body splashes or body sprays. The term “composition” may also include cleaning compositions, such as fabric care composition or home care compositions, including air care compositions (e.g., air fresheners), for use on clothing or other substrates such as hard surfaces (e.g., dishes, floors, countertops). Additional non-limiting examples of “composition” may also include facial or body powder, deodorant, foundation, body/facial oil, mousse, creams (e.g., cold creams), waxes, sunscreens and blocks, bath and shower gels, lip balms, self-tanning compositions, masks and patches.

As used herein, the term “consumer” means both the user of the composition and the observer nearby or around the user.

As used herein, the term “fragrance material” and “fragrance materials” relates to a perfume raw material (“PRM”), or a mixture of perfume raw materials (“PRMs”), that are used to impart an overall pleasant odor or fragrance profile to a composition. “Fragrance materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also known for use as “fragrance materials”. The individual perfume raw materials which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, N.J., USA and more recently re-published by Allured Publishing Corporation Illinois (1994). Additionally, some perfume raw materials are supplied by the fragrance houses (Firmenich, International Flavors & Fragrances, Givaudan, Symrise) as mixtures in the form of proprietary specialty accords. Non-limiting examples of the fragrance materials useful herein include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH-change, or by enzymatic release.

As used herein, the term “fragrance profile” means the description of how the fragrance is perceived by the human nose at any moment in time. The fragrance profile may change over time. It is a result of the combination of the low, moderate and high volatile fragrance materials, if present, of a fragrance. A fragrance profile is composed of 2 characteristics: ‘intensity’ and ‘character’. The ‘intensity’ relates to the perceived strength whilst ‘character’ refers to the odor impression or quality of the perfume, e.g., fruity, floral, woody, etc.

As used herein, the terms “modulator”, and “fragrance modulator” are used interchangeably to designate an agent having the capacity to affect the fragrance profile, such as for example, by impacting the fragrance materials' evaporation rate. The modulator may mediate its effect by lowering the vapor pressure of the fragrance materials and increasing their adherence to the substrate (skin and/or hair) thus ensuring a less harsh impression of the overdosed fragrance.

By incorporating the modulator, it is desired that the fragrance profile, preferably the fragrance components composition attributable to the moderate and low volatile fragrance materials, alone or individually, of the composition can be perceived by a panel of experts or professional evaluators or individual experts or professional evaluators, without the perceived harshness of overdosing (e.g., greater than about 30 wt % of the composition) of the low and moderate fragrance materials is mitigated or absent, as compared to the same perception in the absence of the modulator. Suitable examples of the modulator are provided herein below. However, as discovered by the inventors, simply adding a primer containing modulators to a traditionally constructed fragrance composition (e.g., classical fragrance pyramid construction without overdose) that is commercially available in the market will enhance the perception of medium and high volatile materials and preserve them over time. It is necessary that fragrance construction has a predominantly green, fruity, floral and citrus character to achieve best results. Nonetheless desired character change is achieved with any commercial fragrance as described by experts or professional evaluators or individual experts or professional evaluators experience.

As used herein, the term “substantially non-odorous” means an agent that does not impart an odor of its own when added into a composition of the present invention. For example, a “substantially non-odorous fragrance modulator” does not impart a new odor that alters the character of the fragrance profile of the composition to which it is added. The term “substantially non-odorous” also encompasses an agent that may impart a minimal or slight odor of its own when added into a composition of the present invention. However, the odor imparted by the “substantially non-odorous fragrance modulator” is generally undetectable or tends to not substantively alter the character of the fragrance profile of the composition to which it is added initially or preferably over time. Furthermore, the term “substantially non-odorous” also includes materials that are perceivable only by a minority of people or those materials deemed “anosmic” to the majority of people. Furthermore, the term “substantially non-odorous” also includes materials that may, from particular suppliers, contain an odor due to impurities, such as when the materials contain the impurities at not more than about 5 wt %, preferably not more than 1 wt %, often even not more than 1 part per million (ppm). These impurities maybe removed by purification techniques known in the art as required to make them suitable for use in fragrance compositions of the present invention.

As used herein, the term “vapor pressure” means the partial pressure in air at a defined temperature (e.g., 25° C.) and standard atmospheric pressure (760 mmHg) for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a fragrance material which is defined in an open environment where material is leaving the system. The vapor pressure is determined according to the reference program Advanced Chemistry Development (ACD/Labs) Software Version 14.02, or preferably the latest version update).

It is understood that the test methods that are disclosed in the Test Methods Section of the present application must be used to determine the respective values of the parameters of Applicants' inventions as described and claimed herein.

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety, for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C—C(CH), —C═C(CH₂CH₃), —CH₂C═CH, —CH₂C═C(CH₃), and —CH₂C═C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

### Compositions

Compositions described herein can relate to a coating composition such as a primer or topcoat that includes a substantially non-odorous fragrance modulator. The coating composition can further include a carrier in which the substantially non-odorous fragrance modulator is dispersed. The composition can further include at least one low volatile fragrance, moderate volatile fragrance, high volatile fragrance, or a combination thereof. The fragrance can be present in a range of from about 0.01 wt % to about 50 wt %, about 0.05 wt % to about 25 wt %, less than, equal to, or greater than about 0.01 wt %, 0.05, 0.1, 0.5, 1, 2, 3, 4, 5, 6, 7, 8, 9, 10, 15, 20, 25, 30, 35, 40, 45, or about 50 wt %. The composition can then be added to an existing fragrance composition.

Fragrance Modulators

The coating composition comprises at least one substantially non-odorous fragrance modulator as described herein below. Suitable examples of the substantially non-odorous fragrance modulators are provided in Table 1 below.

The substantially non-odorous fragrance modulator can be present in an amount of from about 0.1 wt % to about 50 wt % relative to the total weight of the coating composition or the composition, about 0.5 wt % to about 40 wt %, about 2.5 wt % to about 15 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50 wt %. If there are more than one substantially non-odorous fragrance modulators, then the ranges provided herein above cover the total of all of the substantially non-odorous fragrance modulators in the composition. According to various embodiments, the modulator can be present exclusively in a primer as opposed to being in a fragrance composition.

The substantially non-odorous fragrance modulator can be a liquid at temperatures lower than 100° C., such as at ambient temperature. The substantially non-odorous fragrance modulators may be fully miscible with a fragrance material(s) to form a single-phase liquid. However, if the fragrance materials are not entirely miscible, or are immiscible, then co-solvents (e.g., dipropylene glycol (DPG), triethyl citrate, or others well known to those skilled in the art) can be added to aid in the solubility of the fragrance materials.

The coating can be used in conjunction with a fragrance composition. For example, the fragrance composition can be applied to the coating and the substantially non-odorous fragrance modulator can permeate to the fragrance component. The coating can be left to dry or be a liquid when a fragrance component is brought into contact with the coating. The effect of the substantially non-odorous fragrance modulator on the fragrance profile, particularly the characters of the fragrance profile which is attributable to the medium and high volatile fragrance materials, can be improved. By “improved” it is meant that the fragrance profile of the composition, particularly the components contributed by at least one of the medium and high volatile fragrance materials, can be perceived by the panel of experts or professional evaluators or individual experts or professional evaluators at later time points such as, for example, 1 hour, 2 hours, 3 hours, 4 hours, 5 hours, 6 hours, 7 hours, 8 hours, 10 hours, and possibly all the way up to 24 hrs after application as compared to controls, e.g., lacking any of the disclosed non-odorous fragrance modulators such as Glucam.

Suitable examples of non-odorous modulators can include methyl glucoside polyol, ethyl glucoside polyol, propyl glucoside polyol, or mixtures thereof. Further examples can include from polypropylene glycol-10 methyl glucose ether, ethoxylated methyl glucose ether, polypropylene glycol-20 methyl glucose ether, caprylyl/capryl glucoside, undecyl glucoside, and mixtures thereof. In some examples, the composition can be substantially free of isocetyl alcohol, diisobutyl adipate, diisoamyl adipate, polypropylene glycol-3 myristyl ether, and neopentyl glycol diethyl hexanoate, neopentyl glycol diisononanoate, cetearyl ethyl hexanoate, and their mixtures, or a mixture thereof, although the composition can optionally include these.

Further examples of non-odorous modulators include:
a compound of formula (I):

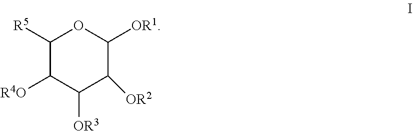

wherein:
   R¹ is hydrogen, alkyl, alkenyl or alkynyl;
   R² is selected from hydrogen, (C₂-C₂₀)alkyl, (C₂-C₂₀)alkenyl, (C₂-C₂₀)alkynyl, —[R⁶R⁷(R⁸)0]_{w}R⁹, wherein w is from 1 to 10, preferably 2 to 9;
   R³ is selected from hydrogen, alkyl, alkenyl, alkynyl, —[R⁶R\R⁸)0]yR⁹, wherein y is
from 1 to 10 or 2 to 9;
   R⁴ is selected from hydrogen, alkyl, alkenyl, alkynyl, —[R⁶R\R⁸)0]xR⁹, wherein x is from 1 to 10, preferably 2 to 9;
   R⁵ is selected from hydrogen, alkyl, alkenyl, alkynyl, —R⁶0 R⁹, —R⁶0 [R⁶R⁷(R⁸)0]zR⁹,
   wherein z is from 1 to 10, preferably 2 to 9;
   each R⁶ and R⁷ are independently selected from (C₂-C₂₀)alkylene, (C₂-C₂₀)alkenylene, or (C₂-C₂₀)alkynylene; and
   each R⁸ and R⁹ is independently selected from hydrogen or alkyl, a compound of formula (II):

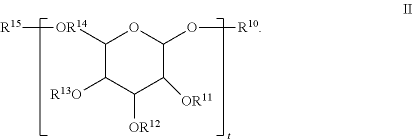

wherein:
   R¹⁰ is hydrogen, (C₂-C₂₀)alkyl, (C₂-C₂₀)alkenyl or (C₂-C₂₀)alkynyl;
   each R¹¹ is independently selected from hydrogen, (C₂-C₂N)alkyl, (C₂-C₂₀)alkenyl, (C₂-C₂₀)alkynyl;
   each R¹² is independently selected from hydrogen, (C₂-C₂N)alkyl, (C₂-C₂₀)alkenyl, or (C₂-C₂₀)alkynyl;
   each R¹³ is independently selected from hydrogen, (C₂-C₂₀)alkyl, (C₂-C₂₀)alkenyl, or (C₂-C₂₀)alkynyl;
   each R¹⁴ is selected from (C₂-C₂₀)alkylene, (C₂-C₂₀)alkenylene, or (C₂-C₂₀)alkynylene; and
   R¹⁵ is hydrogen, (C₂-C₂₀)alkyl, (C₂-C₂₀)alkenyl or (C₂-C₂₀) alkynyl; wherein tis 5 or less, preferably 1, 2 or 3;
Sucrose Laurate, Sucrose Dilaurate, Sucrose Myristate, Sucrose Palmitate, Sucrose Stearate, Sucrose Distearate, Sucrose Tristearate, and their mixtures;
Trimethylcyclohexane derivatives having the formula (III):

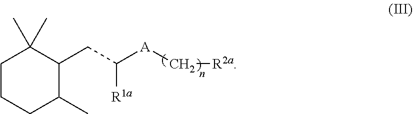

wherein:
   n is 0, 1 or 2;
   A is C═O or CH—OH;
   R^{1a} is hydrogen or methyl;
   R^{2a} is a C₂-C₁₀ hydrocarbon group; and
      is a saturated or unsaturated carbon-carbon bond;
L-menthoxy ether derivatives having the formula (IV):

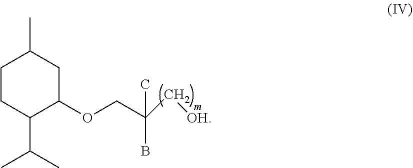

wherein:
   m is 0, 1 or 2;
   B is hydrogen or OH; and
   C is hydrogen or methyl;
Tetra-hydronaphthalene derivatives having the formula (V):

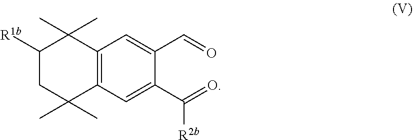

wherein:
   R^{1b} is hydrogen or methyl; and
   R^{2b} is alkyl;
   140
Hyaluronic acid disaccharide sodium salt, sodium hyaluronate and their mixtures;
Ether derivatives having the formula (VI) or formula (VII):

C₅HₗOₘ—(OR^{1c})ₙ (VI).
wherein:
   C₅HₗOₘ is a pentose residue, wherein l is an integer from 6 to 9, and m is an integer from 1 to 4;
   n is an integer from 1 to 4; and
   R^{1c} is C₄-C₂₀ hydrocarbon group; and

C₅H_{c}O_{d}—(OCH₂CH₂—O—CH₂CH₂—O—R^{1c})ₑ (VII).
wherein:
   C₆HₓO_{y} is a hexose residue, wherein x is an integer from 7 to 11, and y is an integer from 1 to 5;
   z is an integer from 1 to 5; and
   R^{1d} is C₄-C₂₀ hydrocarbon group; and
Diethylene Glycol Ether derivatives having the formula (VIII) or formula (IX):

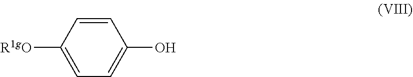

wherein:
   C₅H_{c}O_{d} is a pentose residue, wherein c is an integer from 6 to 8, and d is an integer from 1 to 3;
   e is an integer from 2 to 4; and
   R^{1e} is C₁-C₆ alkyl group; and

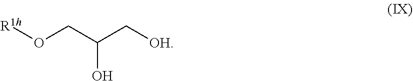

wherein:
   C₆H_{f}O_{g} is a hexose residue, wherein f is an integer from 7 to 10, and g is an integer from 1 to 4;
   h is an integer from 2 to 5; and
   R^{1f} is C₁-C₆ alkyl group;
   Hydroquinone Glycoside derivatives having the formula (X):

R¹ⁱOCOR²ⁱCOOR³ⁱ (X)
wherein:
   R^{1g} is selected from the group consisting of; (i) pentose residue, hexose residue, aminosaccharide residue, uronic acid residue and their mixtures; (ii) methylated versions of group (i); and (iii) mixtures of groups (i) and (ii); and Propylene Glycol Propyl Ether; Dicetyl Ether; Polyglycerin-4 Ethers; Isoceteth-5; Isoceteth-7, Isoceteth-10; Isoceteth-12; Isoceteth-15; Isoceteth-20; Isoceteth-25; Isoceteth-30; Disodium Lauroamphodipropionate; Hexaethylene glycol monododecyl ether; and their mixtures:
Glyceryl Ether derivatives having the formula (XI):

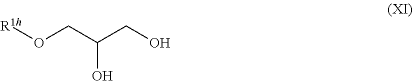

wherein:
   R^{1h} is C₄-C₁₂ aliphatic hydrocarbon group;
Panthenol Ethyl Ether, DL-Panthenol and their mixtures;
Aliphatic Dibasic Acid Diester derivatives having the formula (XII):

R¹ⁱOCOR²ⁱCOOR³ⁱ (XII).
wherein:
   R¹ⁱ is C₄-C₅ alkyl;
   R²ⁱ is C₄ alkylene; and
   R³ⁱ is C₄-C₅ alkyl; and
Aliphatic Ether derivatives having the formula (XIII):

R⁴ⁱ—O—(CH(CH₃)—CH₂O)ₐ(CH₂CH₂O)_{b}—H (XIII).
wherein:
   a and b are integers such that the sum of a and b is from 1 to 4; and
   R⁴ⁱ is an aliphatic chain comprising from 8 to 18 carbons;
N-hexadecyl n-nonanoate, Noctadecyl n-nonanoate and their mixtures;
Tricyclodecane Amide derivatives selected from the group consisting of:
   the compounds of formula (XIV):

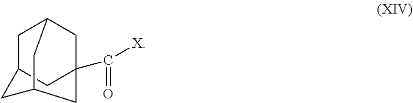

wherein:
   X is selected from:

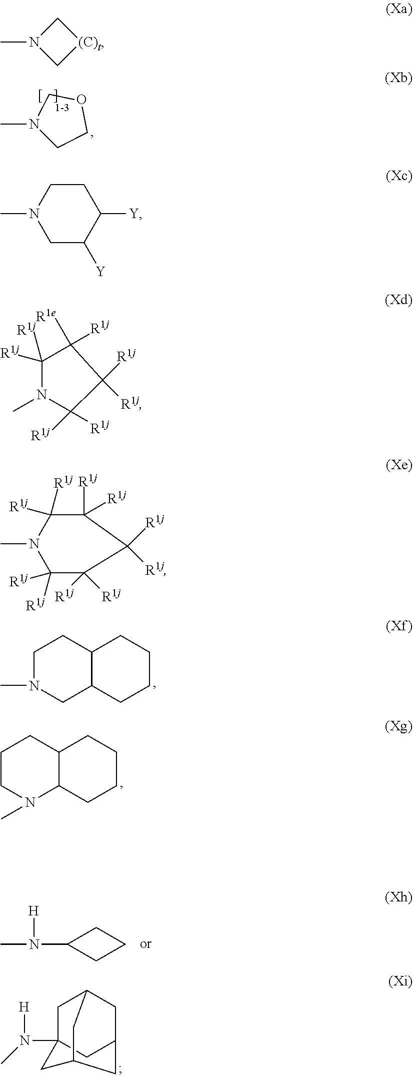

t is 1 to 8;
   Y is hydrogen, or a halogen; and
   each R^{1j} is independently selected from a hydrogen, or C₁-C₄ alkyl;
the compounds of formula (XV):

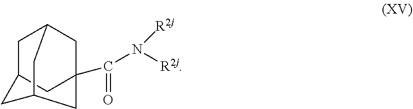

wherein:
   each R^{2j} is independently selected from a hydrogen, methyl, ethyl or C₃-C₁₈ alkyl, cycloalkyl or cycloheteroalkyl, with the proviso that both R^{2e} groups are not hydrogen; and
mixtures of the compounds of formulae (XII) and (XIII); and
mixtures thereof.

Tables 1(a) and 1(b) provide lists of suitable non-odorous fragrance modulators.

TABLE 1(a)
Substantially Non-Odorous Fragrance Modulators

| No. | Group | Chemical Name | CAS Number | Supplier |
|---|---|---|---|---|
| 1. | (a) | PPG-10 Methyl Glucose Ether | 61849-72-7 | Lubrizol |
| 2. | | PPG-20 Methyl Glucose Ether ¹ | 61849-72-7 | |
| 3. | | Ethoxylated Methyl Glucose | 68239-42-9 | |
| | | Ether ² | | |
| 4. | | Caprylyl/Capryl Glucoside ³ | 68515-73-1 | BASF |
| 5. | | Undecyl Glucoside ^{3a} | — | SEPPIC (France) |
| 6. | (b) | Isocetyl Alcohol ⁴ | 36653-82-4 | Ashland Speciality |
| | | | | Ingredients |
| 7. | (c) | PPG-3 Myristyl Ether ⁵ | — | Evonik |
| 8. | | Neopentyl Glycol | 28510-23-8 | Lubrizol |
| | | Diethylhexanoate ⁶ | | |
| 9. | (d) | Sucrose Laurate | 25339-99-5 | Alfa Chemicals Ltd. (UK) |
| 10. | | Sucrose dilaurate | 25915-57-5 | Alfa Chemicals Ltd. (UK) |
| 11. | | Sucrose Myristate | 27216-47-3 | Mitsubishi Chemicals |
| 12. | | Sucrose Palmitate | 26446-38-8 | Alfa Chemicals Ltd. (UK) |
| 13. | | Sucrose Stearate | 25168-73-4 | |
| 14. | | Sucrose Distearate | 27195-16-0 | Mitsubishi Chemicals (JP) |
| 15. | | Sucrose Tristearate | 27923063-3 | Mitsubishi Chemicals (JP) |
| 16. | (e) | (E)-1-(2,2,6- | — | Takasago (Japan) |
| | | trimethylcyclohexyl)oct-1-en-3-one ⁸ | | |
| 17. | (f) | 2-(1-menthoxy)ethane-1-ol ⁹ | — | Takasago (Japan) |
| 18. | | 1-(1-menthoxy)propane-2-ol ⁹ | — | |
| 19. | | 3-(1-menthoxy)propane-1-ol ⁹ | — | |
| 20. | | 3-(1-menthoxy)propane-1,2- | — | |
| | | diol ⁹ | | |
| 21. | | 2-methyl-3-(1- | — | |
| | | menthoxy)propane-1,2-diol ⁹ | | |
| 22. | | 4-(1-menthoxy) butane-1-ol ⁹ | — | |
| 23. | (g) | 1,1,4,4-tetramethy1-6-acetyl-7- | | Givaudan (Switzerland) |
| | | formyl-1,2,3,4- | — | |
| | | tetrahydronaphthalene ¹⁰ | | |
| 24. | | 1,1,2,4,4-pentamethyl-6-acetyl- | — | |
| | | 7-formyl-1,2,3,4- | | |
| | | tetrahydronaphthalene ¹⁰ | | |
| 25. | (h) | Hyaluronic acid disaccharide | 9004-61-9 | Sigma Aldrich (UK) |
| | | sodium salt ¹¹ | | |
| 26. | | Sodium Hyaluronate ¹¹ | 9067-32-7 | |
| 27. | (i) | Mono-o-(linalyl)-glucopyranose ¹² | — | Kanebo (Japan) |
| 28. | | Di-o-(linalyl)-glucopyranose ¹² | — | |
| 29. | | Tri-o-(linalyl)-glucopyranose ¹² | — | |
| 30. | | Tretra-o-(linalyl)-glucopyranose ¹² | — | |
| 31. | | Penta-o-(linayl)-glucopyranose ¹² | — | |
| 32. | | Mono-o-(cis-3-hexenyl)- | — | |
| | | glactopyranose ¹² | | |
| 33. | | Di-o-(cis-3-hexenyl)- | — | |
| | | glactopyranose ¹² | | |
| 34. | | Tri-o-(cis-3-hexenyl)- | — | |
| | | glactopyranose ¹² | | |
| 35. | | Tetra-o-(cis-3-hexenyl)- | — | |
| | | glactopyranose ¹² | | |
| 36. | | Penta-o-(cis-3-hexenyl)- | — | |
| | | glactopyranose ¹² | | |
| 37. | (j) | Bis-O-(3,6-dioxadecanyl)- | — | |
| | | glucopyranose ¹³ | | |
| 38. | | Tris-O-(3,6-dioxadecanyl)- | — | |
| | | glucopyranose ¹³ | | |
| 39. | | Tetrakis-O-(3,6-dioxadecanyl)- | — | |
| | | glucopyranose ¹³ | | |
| 40. | | Pentakis-O-(3,6-dioxadecanyl)- | — | |
| | | glucopyranose ¹³ | | |
| 41. | | Bis-O-(3,6-dioxaoctanyl)- | — | |
| | | galactopyranose ¹³ | | |
| 42. | | Tris-O-(3,6-dioxaoctanyl)- | — | |
| | | galactopyranose ¹³ | | |
| 43. | | Tetrakis-O-(3,6-dioxaoctanyl)- | — | |
| | | galactopyranose ¹³ | | |
| 44. | | Pentakis-O-(3,6-dioxaoctanyl)- | — | |
| | | galactopyranose ¹³ | | |
| 45. | | Bis-O-(3,6-dioxaheptanyl)- | — | |
| | | xylopyranose ¹³ | | |
| 46. | | Tris-O-(3,6-dioxaheptanyl)- | — | |
| | | xylopyranose ¹³ | | |
| 47. | | Tetrakis-O-(3,6-dioxaheptanyl)- | — | |
| | | xylopyranose ¹³ | | |
| 48. | | Bis-O-(3,6-dioxadodecanyl)- | — | |
| | | glucopyranose ¹³ | | |
| 49. | | Tris-O-(3,6-dioxadodecanyl)- | — | |
| | | glucopyranose ¹³ | | |
| 50. | | Tetrakis-O-(3,6-dioxadodecanyl)- | — | |
| | | glucopyranose ¹³ | | |
| 51. | | Pentakis-O-(3,6-dioxadodecanyl)- | — | |
| | | glucopyranose ¹³ | | |
| 52. | (k) | Hydroquinone beta-D-glycoside ¹⁴ | 497-76-7 | Shiseido |
| 53. | (l) | Propylene Glycol Propyl Ether | 1569-01-3 | Sigma Aldrich (UK) |
| 54. | | Dicetyl Ether | 4113-12-6 | |
| 55. | | Propyglycerin-4 Ethers | 25618-55-7 | Solvay Chemicals |
| 56. | | Isoceteth-5 | 69364-63-2 | Nihon Emulsion |
| 57. | | Isoceteth-7 | 69364-63-2 | Company Ltd. |
| 58. | | Isoceteth-10 | 69364-63-2 | |
| 59. | | Isoceteth-12 | 69364-63-2 | |
| 60. | | Isoceteth-15 | 69364-63-2 | |
| 61. | | Isoceteth-20 | 69364-63-2 | |
| 62. | | Isoceteth-25 | 69364-63-2 | |
| 63. | | Isoceteth-30 | 69364-63-2 | |
| 64. | | Disodium | 68929-04-4 | Rhodia |
| | | Lauroamphodipropionate | | |
| 65. | | Hexaethylene glycol | 3055-96-7 | Sigma Aldrich (UK) |
| | | monododecyl ether ^{14b} | | |
| 66. | (m) | Neopentyl Glycol | 27841-07-2 | Symrise (Germany) |
| | | Diisononanoate ¹⁵ | | |
| 67. | | Cetearyl Ethylhexnoate ¹⁶ | 90411-68-0 | |
| 68. | (n) | 2-ethylhexyloxypropanediol ¹⁷ | 70455-33-9 | Takasago (JP) |
| 69. | (o) | Panthenol Ethyl Ether ¹⁸ | 667-83-4 | DSM Nutritional |
| | | | | Products, Inc. (USA) |
| 70. | | DL-Panthenol | 16485-10-2 | Roche Inc. (USA) |
| 71. | (p) | Diisobutyl Adipate ¹⁹ | 141-04-8 | Sigma Aldrich (UK) |
| 72. | | Diisoamyl Adipate ¹⁹ | 6624-70-0 | |
| 73. | (q) | PPG-11 Stearyl Ether ^{19a} | 25231-21-4 | Kao (JP) |
| 74. | (r) | N-hexadecyl n-nonanoate ^{19b} | 72934-15-7 | Symrise (Germany) |
| | | (e.g., cetyl nonanoate) | | |
| 75. | | Noctadecyl n-nonanoate ^{19b} | 107647-13-2 | |
| | | (e.g., stearyl nonanoate) | | |
| 76. | (s) | methanone, (morphonyl) | — | Unilever (UK) |
| | | tricyclo[3.3.1.1^{3,7}]dec-1-yl- ²⁰ | | |
| 77. | | methanone, (piperidinyl) | — | |
| | | tricyclo[3.3.1.1^{3,7}]dec-1-yl- ²⁰ | | |
| 78. | | methanone, (pyrrolidinyl) | — | |
| | | tricyclo[3.3.1.1^{3,7}]dec-1-yl ²⁰ | | |
| 79. | | methanone, (azetidinyl) | — | |
| | | tricyclo[3.3.1.1^{3,7}]dec-1-yl- ²⁰ | | |
| 80. | | methanone, (hexahydroazepinyl) | — | |
| | | tricyclo[3.3.1.1^{3,7}]dec-1-yl- ²⁰ | | |
| 81. | | methanone, (4-cyano- | — | |
| | | piperidinyl)tricyclo[3.3.1.1^{3,7}]dec- | | |
| | | 1-yl- ²⁰ | | |
| 82. | | methanone, 4-amido- | — | |
| | | piperidinyl)tricyclo[3.3.1.1^{3,7}]dec- | | |
| | | 1-yl- ²⁰ | | |
| 83. | | methanone, | — | |
| | | (Tricyclo[3.3.1.1^{3,7}]decanyl)-N- | | |
| | | tricyclo[3.3.1.1^{3,7}]dec-1-yl- ²⁰ | | |
| 84. | | methanone, | — | |
| | | (decahydroisoquinolinyl)tri- | | |
| | | cyclo[3.3.1.1^{3,7}]dec-1-yl- ²⁰ | | |
| 85. | | methanone, | — | |
| | | (decahydroisoquinolinyl)tri- | | |
| | | cyclo[3.3.1.1^{3,7}]dec-1-yl- ²⁰ | | |
| 86. | | methanone, | — | |
| | | (decahydroquinolinyl)tri- | | |
| | | cyclo[3.3.1.1^{3.7}]dec-1-yl- ²⁰ | | |
| 87. | | methanone, (3,3-dimethyl-1- | — | |
| | | piperidinyl)tricyclo[3.3.1.1^{3,7}] | | |
| | | dec-1-yl- ²⁰ | | |
| 88. | | methanone, (2-methyl-1- | — | |
| | | piperidinyl)tricyclo[3.3.1.1^{3,7}] | | |
| | | dec-1-yl- ²⁰ | | |
| 89. | | methanone, (4-methyl-1- | — | |
| | | piperidinyl)tricyclo[3.3.1.1^{3,7}] | | |
| | | dec-1-yl- ²⁰ | | |
| 90. | | methanone, (3-methyl-1- | — | |
| | | piperidinyl)tricyclo[3.3.1.1^{3,7}] | | |
| | | dec-1-yl- ²⁰ | | |
| 91. | | methanone, (3,5-dimethyl-1- | — | |
| | | piperidinyl)tricyclo[3.3.1.1^{3,7}] | | |
| | | dec-1-yl- ²⁰ | | |
| 92. | | methanone, (4-methyl-4-ethy- | — | |
| | | piperidinyl)tricyclo[3.3.1.1^{3,7}] | | |
| | | dec-1-yl- ²⁰ | | |
| 93. | | methanone, (3,3-diethyl-1- | — | |
| | | pyrrolidinyl)tricyclo[3.3.1.1^{3,7}] | | |
| | | dec-1-yl- ²⁰ | | |
| 94. | | methanone, (N,N-diisopropyl) | — | |
| | | tricyclo[3.3.1.1^{3,7}]dec-1-yl- ²⁰ | | |
| 95. | | methanone, (3,3- | — | |
| | | dimethylbutylaminyl) | | |
| | | tricyclo[3.3,1.1^{3,7}]dec-1-yl- ²⁰ | | |
| 96. | | methanone, (2,2- | — | |
| | | dimethylpropylaminyl) | | |
| | | tricyclo[3.3.1.1^{3,7}]dec-1-yl- ²⁰ | | |
| 97. | | methanone, (1,1-dimethy1-3,3- | — | |
| | | dimethylbutylaminyl) | | |
| | | tricyclo[3.3.1.1^{3,7}]dec-1-yl- ²⁰ | | |
| 98. | | methanone, (1,3-dimethyl- | — | |
| | | butylaminyl) | | |
| | | tricycle[3.3.1.1^{3,7}]dec-1-yl- ²⁰ | | |
| 99. | (t) | Bis-methoxy PEG-13 PEG- | 936645-35-1 | PolymerExpert |
| | | 438/PPG-110 SMDI Copolymer ²¹ | | S.A. (Pessac, France) |
| 100. | (u) | propyl {4-[2-(diethylamino)-2- | 61791-12-6 | Sigma Aldrich (US) |
| | | oxoethoxy]-3- | | |
| | | methoxyphenyl}acetate ²² | | |
| 101. | (v) | 3-((2-ethylhexyl)oxy)propane- | 70445-33-9 | — |
| | | 1,2-diol ²³ | | |
| 102. | | 3-((2-propylheptyl)oxy)propane- | — | — |
| | | 1,2-diol ²³ | | |
| 103. | | 1-amino-3-((2- | 99509-00-9 | — |
| | | ethylhexyl)oxy)propan-2-ol ²³ | | |

¹ available as GLUCAM ™ P-20.
² available as Glucam ™ E-20.
³ available as Plantacare ® 810 UP.
^{3a} available as Simulsol ® SL 11W.
⁴ available as CERAPHYL ® ICA.
⁵ available as Tegosoft ® APM.
⁶ available as Schercemol ™ NGDO.
⁷ disclosed in U.S. Pat. No. 6,737,396B2 (Firmenich), column 1, lines 43-47.
⁸ diclosed as compound 1'i in U.S. Pat. No. 6,440,400B1 (Takasago Int. Corp.), col. 5.
^{8a} diclosed in U.S. Pat. No. 4,313,855 (Dragoco Gerberding & Co. GmbH), col. 1, lines 12-13.
⁹ disclosed in U.S. Pat. No. 7,538,081B2 (Takasago Int. Corp.), column 7, lines 50-53.
¹⁰ disclosed in U.S. Pat. No. 6,147,049 (Givaudan Roure), col. 5, line 24, to col. 6, line 17.
¹¹ disclosed in PCT Publication No. WO85/04803 (Diagnostic), pg. 2, line 1 to pg. 4, line 2.
¹² disclosed in JP Patent No. 61-083114 (Kanebo).
¹³ disclosed in JP Patent No. 61-063612 (Kanebo).
¹⁴ disclosed in JP Patent No. 62-084010 (Shiseido).
^{14b} available as: Laureth-6.
¹⁵ disclosed in U.S. Pat. Publication No. 2011/0104089A1 (Symrise), para. [0001].
¹⁶ available as PCL-Liquid ® 100.
¹⁷ disclosed in U.S. Pat. No. 7,196,052 (Takasago Int. Corp.), col. 4, lines 34-35.
¹⁸ disclosed in EP Patent Publication No. 616800A2 (Givaudan), pg. 2, lines 12-25.
¹⁹ disclosed in U.S. Pat. No. 4,110,626 (Shiseido), column 3, lines 54-56.
^{19a} disclosed in PCT Publication No. WO2014/155019 (LVMH).
^{19b} disclosed in U.S. Pat. No. 9,050,261 (Symrise).
²⁰ disclosed as compounds C1-C22 in WO2014/139952 (Unilever).
²¹ available as Expert Gel ® EG56.
²² available as Kolliphor ® EL.
²³ disclosed in U.S. Pat. No. 9,050,261 (Symrise).

Further examples of non-odorous fragrance modulator is selected from the group of materials disclosed in Table 1(b).

TABLE 1(b)
Substantially Non-Odorous Fragrance Modulators

| | Chemical or | | | |
|---|---|---|---|---|
| No. | INCI Name | Trade Name | CAS Number | Supplier |
| 1. | C12-14 Sec- | Tergitol ® | 68131-40-8 | Sigma Aldrich (UK) |
| | Pareth-3 | 15-S-7 | | |
| 2. | Poly(ethylene | PPG-7- | 9038-95-3 | Sigma Aldrich (UK) |
| | glycol-ran- | Buteth-10 | | |
| | propylene glycol) | | | |
| | monobutyl ether | | | |
| 3. | PPG-4-Ceteth-10 | Nikkol PBC-33 | 37311-01-6 | Chemical Navi |
| 4. | Deceth-4 | Ethal DA-4 | 5703-94-6 | Ethox Chemicals, |
| | | | | Inc. |
| 5. | PPG-5-Ceteth-20 | AEC PPG-5- | 9087-53-0 | A & E Connock |
| | | Ceteth-20 | | (Perfumery & |
| | | | | Cosmetics) Ltd. |
| 6. | C14-15 Pareth-7 | Neodol 45-7 | 68951-67-7 | Shell Chemical |
| | | alcohol | | Company |
| | | ethoxylate | | |
| 7. | Linear alcohol | Bio-soft N25-7 | 68131-39-5 | Stephan |
| | (C12-15) Pareth- | | | Company (USA) |
| | 3ethoxylate, | | | |
| | POE-7 | | | |
| 8. | Linear alcohol | Bio-soft N23-6.5 | 66455-14-9 | |
| | (C12-13) Pareth- | | | |
| | 3ethoxylated, | | | |
| | POE-6.5) | | | |
| 9. | Polyethylene | Cremophor ® A 25 | 68439-49-6 | Sigma Aldrich (UK) |
| | glycol 1100 | | | |
| | mono(hexadecyl/ | | | |
| | octadecyl) ether | | | |
| 10. | Linear alcohol | Bio-soft N91-8 | 68439-46-3 | Stephan |
| | (C9-11) | | | Company (USA) |
| | ethoxylated | | | |
| | POE -8 Pareth-3 | | | |
| 11. | Coceth-10 or | Genapol ® C-100 | 61791-13-7 | Sigma Aldrich (UK) |
| | Polyoxyethylene | | | |
| | (10) dodecyl ether | | | |
| 12. | Alcohols, C12-14, | Rhodasurf ® LA 30 | 68439-50-9 | Solvay Solutions |
| | ethoxylated | | | Italia S.p.A. |
| 13. | Poly(ethylene | Poly(ethylene | 9004-74-4 | Sigma Aldrich (UK) |
| | glycol) | glycol) | | |
| | methyl ether | methyl ether | | |
| 14. | C10-16 Pareth-1 | Neodol ® PC 110 | 68002-97-1 | Shell Chemical |
| | | | | Company |
| 15. | PPG-11 Stearyl | Arlamol ™ PS11E | 25231-21-4 | Croda (UK) |
| | Ether | | | |
| 16. | Steareth-100 | Brij ® S100 | 9005-00-9 | Sigma Aldrich (UK) |
| 17. | Polyethylene | Brij ® C-58 | 9004-95-9 | Sigma Aldrich (UK) |
| | glycol hexadecyl | | | |
| | ether | | | |
| 18. | Pluronic ® F-127 | Pluronic ® F-127 | 9003-11-6 | Sigma Aldrich (UK) |
| 19. | Linear Alcohol | Bio-soft N1-5 | 34398-01-1 | Stepan Canada Inc. |
| | (C11) Ethoxylate, | | | |
| | POE-5 | | | |
| 20. | Laureth-10 | Intrasol FA | 6540-99-4 | Evonik Industries |
| | | 12/18/10 | | AG |
| 21. | Decaethylene | Polyoxyethylene | 9002-92-0 | Sigma Aldrich (UK) |
| | glycol | (10) lauryl ether | | |
| | mono-dodecyl | | | |
| | ether | | | |
| 22. | Ethylene glycol | 2- | 109-86-4 | Sigma Aldrich (UK) |
| | monomethyl ether | Methoxyethanol | | |
| 23. | Myreth-4 | Homulgator | 27306-79-2 | Grau Aromatics |
| | | 920 G | | GmbH & |
| | | | | Company KG |
| 24. | Oleth-16 | Pegnol O-16A | 25190-05-0 | Toho Chemical |
| | Alkoxylated | | | Industry Co., Ltd. |
| | Alcohols | | | |
| 25. | Isosteareth-5 | Emalex 1805 | 52292-17-8 | Nihon Emulsion |
| | | | | Company, Ltd. |
| 26. | PPG-10 Cetyl | Arlamol ™ PC10 | 9035-85-2 | Croda (UK) |
| | Ether | | | |
| 27. | Polyoxy(ethylene | Poly(ethylene | 24938-91-8 | Sigma-Aldrich (UK) |
| | glycol) (18) | glycol) (18) | | |
| | tridecyl ether | tridecyl ether | | |
| 28. | Poly(oxy-1,2- | ALFONIC ® 10-8 | 26183-52-8 | Sasol Chemicals |
| | ethanediyl), | Ethoxylate | | (USA) LLC |
| | a-decyl- | | | |
| | w-hydroxy- | | | |
| 29. | Laureth-1 | Mackam ™ 2LSF | 4536-30-5 | Rhodia (DE) |
| 30. | PEG-5 | Ethox HTAM-5 | 61791-26-2 | Ethox Chemicals, |
| | Hydrogenated | | | Inc. |
| | Tallow Amine | | | |
| 31. | PEG-15 Oleamine | Nikkol | 26635-93-8 | Nikko Chemicals |
| | | TAMNO-15 | | Co., Ltd. |
| 32. | Polyoxyethylene | Brij ® O20-SS | 9004-98-2 | Sigma Aldrich (UK) |
| | (20) oleyl ether | | | |
| 33. | Cetoleth-10 | Brij ® CO10 | 8065-81-4 | Croda, Inc. |
| 34. | Talloweth-7 | Emulmin 70 | 61791-28-4 | Sanyo Chemical |
| | | | | Industries Ltd. |
| 35. | Isobutoxypropanol | Isobutoxypropanol | 34150-35-1 | MolPort |
| | Alcohols | | | |
| 36. | Isobutoxypropanol | Isobutoxypropanol | 23436-19-3 | AKos Consulting |
| | Alcohols | | | & Solutions |
| 37. | Diethylene Glycol | Twincide EDG | 111-46-6 | Roda |
| 38. | Methoxyethanol | Hisolve MC | 109-86-4 | Tolio Chemical |
| | | | | Industry Co., Ltd. |
| 39. | Ethoxyethanol | 2-Ethoxyethanol | 110-80-5 | Sigma-Aldrich (UK) |
| | Alcohols | | | |
| 40. | Methoxyisopropanol | Dowanol ™ PM | 107-98-2 | The Dow |
| | Alcohols | | | Chemical |
| | | | | Company |
| 41. | Methoxyethanol | Hisolve MC | 32718-54-0 | Toho Chemical |
| | | | | Industry Co., Ltd. |
| 42. | Methylal | Dimethoxymethane | 109-87-5 | Sigma-Aldrich (UK) |
| | Ethers | | | |
| 43. | 3-Methoxybutanol | Methoxybutanol | 2517-43-3 | Hans |
| | | | | Schwarzkopf |
| | | | | GmbH/Co. KG |
| 44. | Butoxyethanol | Butyl OXITOL | 111-76-2 | Shell Chemical |
| | | | | Company |
| 45. | Propylene Glycol | Dowanol ™ PnB | 5131-66- | The Dow |
| | n-Butyl Ether | | 8/29387- | Chemical |
| | | | 86-8 | Company |
| 46. | Propylene Glycol | Propylene Glycol | 15821-83-7 | Sigma Aldrich (UK) |
| | Butyl Ether | Butyl Ether | | |
| 47. | 2-(2- | Diethylene glycol | 112-34-5 | Sigma Aldrich (UK) |
| | butoxyethoxy)ethanol | butyl ether | | |
| 48. | Deceth-4 Phosphate | Crodafos ™ D4A | 52019-36-0 | Croda, Inc. |
| 49. | 2- | Ethylene glycol | 2136-71-2 | Sigma-Aldrich (UK) |
| | (Hexadecyloxy)ethanol | monohexadecyl | | |
| | | ether | | |
| 50. | Poly(propylene | Poly(propylene | 9003-13-8 | Sigma-Aldrich (UK) |
| | glycol) | glycol) | | |
| | monobutyl ether | monobutyl ether | | |
| 51. | Propylene Glycol | Dowanol ™ PnP | 30136-13-1 | The Dow |
| | Propyl Ether | | | Chemical |
| | | | | Company |
| 52. | Propylene Glycol | Dowanol ™ PnB | 29387-86- | The Dow |
| | n-Butyl Ether | | 8/5131-66-8 | Chemical |
| | | | | Company |
| 53. | Dipropylene glycol | Di(propylene | 34590-94-8 | Sigma Aldrich (UK) |
| | monomethyl ether | glycol) methyl | | |
| | | ether, mixture | | |
| | | of isomers | | |
| 54. | Dipropylene Glycol | Proglyde ™ DMM | 111109-77-4 | The Dow |
| | Dimethyl Ether | | | Chemical |
| | | | | Company |
| 55. | PPG-2 Methyl Ether | Dowanol ™ DPM | 13429-07-7 | The Dow |
| | | | | Chemical |
| | | | | Company |
| 56. | Methoxydiglycol | OriStar | 111-77-3 | Orient Stars LLC |
| | Ethers | DEGME | | |
| 57. | Diethylene glycol | Di(ethylene | 111-90-0 | Sigma Aldrich (UK) |
| | ethyl ether | glycol) ethyl | | |
| | | ether | | |
| 58. | Dimethoxydiglycol | Dimethyldiglycol | 111-96-6 | H & V Chemicals |
| | Ethers | | | |
| 59. | PPG-3 Methyl Ether | Dowanol ™ TPM | 37286-64-9 | The Dow |
| | | | | Chemical |
| | | | | Company |
| 60. | Methyl Morpholine | 224286 | 7529-22-8 | Sigma-Aldrich (UK) |
| | Oxide Amine Oxides | ALDRICH | | |
| | | 4-Methylmorpholine | | |
| | | N-oxide | | |
| 61. | Oleth-3 | Brij ® O3 | 5274-66-8 | Croda Europe, Ltd. |
| 62. | Tri(propylene | Dowanol ™ TPnB | 55934-93-5 | Sigma-Aldrich (UK) |
| | glycol) | | | |
| | n-butyl ether | | | |
| 63. | Tripropylene Glycol | Tripropylene Glycol | 24800-44-0 | Sigma-Aldrich (UK) |
| 64. | PPG-3 Methyl Ether | Dowanol ™ TPM | 25498-49-1 | The Dow |
| | Alkoxylated Alcohols | | | Chemical |
| | | | | Company |
| 65. | Triethylene glycol | Triglycol | 112-27-6 | Sigma Aldrich (UK) |
| 66. | PEG-3 Methyl Ether | Hymol ™ | 112-35-6 | Toho Chemical |
| | | | | Industry Co., Ltd. |
| 67. | Laureth-3 | AEC Laureth-3 | 3055-94-5 | A & E Connock |
| | | | | (Perfumery & |
| | | | | Cosmetics) Ltd. |
| 68. | Ethylhexylglycerin | AG-G-75008 | 70445-33-9 | Angene Chemical |
| 69. | Tetra(ethylene | Tetraethylene | 112-60-7 | Sigma Aldrich (UK) |
| | glycol) | glycol | | |
| 70. | Steareth-3 | Isoxal 5 | 4439-32-1 | Vevy Europe SpA |
| 71. | Ceteth-3 | Emalex 103 | 4484-59-7 | Nihon Emulsion |
| | | | | Company, Ltd. |
| 72. | Myreth-3 | Isoxal 5 | 26826-30-2 | Vevy Europe SpA |
| 73. | Trideceth-3 | Alfonic ® TDA-3 | — | Sasol North |
| | | Ethoxylate | | America, Inc. |
| 74. | Ceteth-2 | Brij ® C2 | 5274-61-3 | Croda Europe, Ltd. |
| 75. | Oleth-2 | Brij ® O2 | 5274-65-7 | Croda, Inc. |
| 76. | Steareth-2 | Brij ® S2 | 16057-43-5 | Croda, Inc. |
| 77. | Cetoleth-10 | Brij ® CO10 | 8065-81-4 | Croda, Inc. |
| 78. | Trimethyl Pentanol | Trimethyl Pentanol | 68959-25-1 | Angene Chemical |
| | Hydroxyethyl | Hydroxyethyl | | |
| | Ether Alcohols | Ether | | |
| 79. | Steareth-10 Allyl Ether | Salcare ® SC80 | 109292-17-3 | BASF |
| 80. | TEA-Lauryl Ether | material ID- | 1733-93-3 | Angene Chemical |
| | | AG-J-99109 | | |
| 81. | Polyglyceryl-2 | Chimexane NB | 71032-90-1 | Chimex |
| | Oleyl Ether | | | |
| 82. | Batyl Alcohol | B402 | 544-62-7 | Sigma-Aldrich (UK) |
| | | ALDRICH | | |
| 83. | Octaethylene Glycol | 15879 | 5117-19-1 | Sigma-Aldrich (UK) |
| | | ALDRICH | | |
| 84. | Triglycerol | Cithrol ™ | 66082-42-6 | Croda (UK) |
| | diisostearate | | | |
| 85. | Diglycerin | Diglycerin 801 | 59113-36-9 | Sakamoto |
| | | | | Yakuhin Kogyo |
| | | | | Co., Ltd. |
| 86. | Polyglycerin #310 | Polyglycerin #310 | 25618-55-7 | Sakamoto |
| | | | | Yakuhin Kogyo |
| | | | | Co., Ltd. |
| 87. | Distearyl Ether | Cosmacol ® SE | 6297-03-6 | Sasol Germany |
| | | | | GmbH |
| 88. | Caprylyl Glyceryl | Caprylyl Glyceryl | 10438-94-5 | AKos Consulting |
| | Ether | Ether | | & Solutions |
| 89. | Chimyl Alcohol | Chimyl Alcohol | 506-03-6 | Nikko Chemicals |
| | | | | Co., Ltd. |
| 90. | Dipentaerythrityl | Liponate ® DPC-6 | 68130-24-5 | Lipo Chemicals, |
| | Hexacaprylate/ | | | Inc. |
| | Hexacap rate | | | |
| 91. | Morpholine | 394467 | 110-91-8 | Sigma-Aldrich (UK) |
| | | ALDRICH | | |
| 92. | Dimethyl Oxazolidine | OXABAN ™-A | 51200-87-4 | The Dow |
| | | | | Chemical |
| | | | | Company |
| 93. | Ethyl Hydroxymethyl | 4- | 68140-98-7 | Angene Chemical |
| | Oleyl Oxazoline | Oxazolemethanol | | |
| 94. | Methyl Hydroxymethyl | Adeka Nol GE-RF | 14408-42-5 | Adeka |
| | Oleyl Oxazoline | | | Corporation |
| 95. | Pramoxine HCl | OriStar | 637-58-1 | Orient Stars LLC |
| | | PMHCL | | |
| 96. | Allantoin Ascorbate | Allantoin Ascorbate | 57448-83-6 | ABI Chem |
| 97. | Stearamidopropyl | Mackalene ™ 326 | 55852-14-7 | Rhodia Inc. |
| | Morpholine Lactate | | | |
| 98. | Dioxolane | Elcotal DX | 646-06-0 | Lambiotte & |
| | | | | CIE S.A. |
| 99. | Glycerol Formal | Glycerol Formal | 5464-28-8 | Sigma Aldrich (UK) |
| 100. | Stearamidopropyl | Mackine 321 | 55852-13-6 | Rhodia Inc. |
| | Morpholine | | | |
| 101. | 2,4,6- | Poly(melamine- | 68002-20-0 | Sigma-Aldrich (UK) |
| | Tris[bis(methoxy- | co- | | |
| | methyl)amino]- | formaldehyde) | | |
| | 1,3,5-triazine | methylated | | |
| 102. | Poloxamine 1307 | Pluracare ® 1307 | 11111-34-5 | BASF |
| 103. | Nonoxynol-8 | Igepal ® CO-610 | 27177-05-5 | Rhodia Inc. |
| 104. | Nonoxynol-10 | Igepal ® CO-710 | 27177-08-8 | Rhodia Inc. |
| 105. | Octoxynol-10 | Nikkol OP-10 | 2315-66-4 | Nikko Chemicals |
| | | | | Co., Ltd. |
| 106. | Nonoxynol-9 | Igepal ® CO-630 | 68987-90-6 | Rhodia Inc. |
| 107. | Nonoxynol-9 | Nonoxynol-9 | 94349-40-3 | Angene Chemical |
| | Iodine | iodine | | |
| 108. | Octylphenoxy | Igepal ® CA-630 | 68987-90-6 | Rhodia Inc. |
| | poly(ethyleneoxy)ethanol, | | | |
| | branched | | | |
| 109. | Sodium Octoxynol-2 | Triton ™ X-200 | 55837-16-6 | The Dow |
| | Ethane Sulfonate | | | Chemical |
| | | | | Company |
| 110. | Benzylhemiformal | Preventol D2 | 14548-60-8 | Lanxess |
| | | | | Corporation |
| 111. | Nonoxynol-2 | Igepal ® CO-210 | 27176-93-8 | Rhodia Inc. |
| 112. | Octoxynol-3 | Igepal ® CA-420 | 2315-62-0 | The Dow |
| | | | | Chemical |
| | | | | Company |
| 113. | Nonoxynol-3 | Marlophen NP 3 | 27176-95-0 | Sasol Germany |
| | | | | GmbH |
| 114. | Alkoxylated | Alkasurf NP-4 | 7311-27-5 | Rhodia Inc. |
| | Alcohols | | | |
| 115. | Nonoxynol-3 | Triethylene | 51437-95-7 | Santa Cruz |
| | | Glycol Mono(p- | | Biotechnology |
| | | nonylphenyl) | | |
| | | Ether | | |
| 116. | Nonoxynol-7 | Lowenol 2689 | 27177-03-3 | Jos. H |
| | | | | Lowenstein & |
| | | | | Sons, Inc. |
| 117. | Nonoxynol-6 | Igepal ® CO-530 | 27177-01-1 | Rhodia Inc. |
| 118. | Nonoxynol-5 | Igepal ® CO-520 | 20636-48-0 | Rhodia Inc. |
| 119. | Nonoxynol-5 | Igepal ® CO-520 | 26264-02-8 | Rhodia Inc. |
| 120. | Nonoxynol-5 | Alkasurf NP-4 | 27176-97-2 | Rhodia Inc. |
| 121. | Polyglyceryl-10 | Nikkol | 102051-00-3 | Nikko Chemicals |
| | Trioleate | Decaglyn 3-OV | | Co., Ltd. |
| 122. | Polyglyceryl-10 | Nikkol | 33940-99-7 | Nikko Chemicals |
| | Dioleate | Decaglyn 2-O | | Co., Ltd. |
| 123. | Polyglyceryl-10 | Caprol 10G40 | 34424-98-1 | Abitec |
| | Tetraoleate | | | Corporation |
| 124. | Polyglyceryl-10 | Nikkol | 79777-30-3 | Nikko Chemicals |
| | Stearate | Decaglyn | | Co., Ltd. |
| | | 1-SVEX | | |
| 125. | Polyglyceryl-10 | S-Face O-1001 P | 79665-93-3 | Sakamoto |
| | Oleate | | | Yakuhin Kogyo |
| | | | | Co., Ltd. |
| 126. | Polyglyceryl-10 | Nikkol | 87390-32-7 | Nikko Chemicals |
| | Myristate | Decaglyn | | Co., Ltd. |
| | | 1-MVEX | | |
| 127. | Dermofeel ® | Dermofeel ® | 34406-66-1 | Dr. Straetmans |
| | G 10 L | G 10 L | | |
| 128. | Polyglyceryl-6 | NIKKOL | 51033-38-6 | Chemical Navi |
| | Laurate | Hexaglyn 1-L | | |
| 129. | Polyglyceryl-6 | S-Face IS-601 P | 126928-07-2 | Sakamoto |
| | Isostearate | | | Yakuhin Kogyo |
| | | | | Co., Ltd. |
| 130. | Choleth-10 | Emalex CS-10 | 27321-96-6 | Nihon Emulsion |
| | | | | Company, Ltd. |
| 131. | Steareth-10 Allyl | Salcare ® SC80 | 109292-17-3 | BASF |
| | Ether/Acrylates | | | |
| | Copolymer | | | |
| 132. | Polyvinyl Stearyl | Giovarez ®1800 | 9003-96-7 | Phoenix |
| | Ether | | | Chemical, Inc. |
| 133. | Dicetyl Ether | Cosmacol Ether 16 | — | Sasol Germany |
| | | | | GmbH |
| 134. | PPG-23-Steareth-34 | Unisafe 34S-23 | 9038-43-1 | Pola Chemical |
| | | | | Industries, Inc. |
| 135. | Stearoxypropyl | Farmin DM E-80 | 17517-01-0 | Kao Corp. |
| | Dimethylamine | | | |
| 136. | Distearyl Ether | Cosmacol SE | 6297-03-6 | Sasol Germany |
| | | | | GmbH |
| 137. | Polyquaternium-10 | AEC | 55353-19-0 | A & E Connock |
| | | Polyquaternium-10 | | (Perfumery & |
| | | | | Cosmetics) Ltd. |
| 138. | Octyl ether | Dioctyl ether | 629-82-3 | Sigma Adirich (UK) |
| 139. | Ethyl Ether | Diethyl Ether | 60-29-7 | EMD Chemicals |
| 140. | Methyl Hexyl | methyl hexyl | 4747-07-3 | TCI AMERICA |
| | Ether Ethers | ether | | |
| 141. | Ceteth-12 | Emalex 112 | 94159-75-8 | Nihon Emulsion |
| | | | | Company, Ltd. |
| 142. | Ceteth-10 or cetyl | Jeecol CA-10 | 14529-40-9 | Jeen |
| | alcohol POE-10 | | | International |
| 143. | Steareth-10 | Jeecol SA-10 | 13149-86-5 | Jeen |
| | | | | International |
| 144. | Nonaethylene glycol | Nonaethylene glycol | 3055-99-0 | Sigma Aldrich (UK) |
| | monododecyl ether | monododecyl ether | | |
| 145. | Oleth-10 | Brij ® O10 | 71976-00-6 | Croda, Inc. |
| 146. | Oleth-10 | Brij ® O10 | 24871-34-9 | Croda, Inc. |
| 147. | PEG-12 | Carbowax ™ | 6790-09-6 | The Dow |
| | | PEG 600 | | Chemical |
| | | | | Company |
| 148. | PEG-9 | Sabopeg 400 | 3386-18-3 | Sabo s.p.a. |
| 149. | PEG-10 | DECAETHYLENE | 5579-66-8 | MolPort |
| | | GLYCOL | | |
| 150. | PEG-6 | Carbowax ™ | 2615-15-8 | The Dow |
| | | PEG 300 | | Chemical |
| | | | | Company |
| 151. | Glycerol | Glycerol | 25791-96-2 | Sigma Aldrich (UK) |
| | propoxylate | propoxylate | | |
| 152. | Glycerol | Glycerol | 31694-55-0 | Sigma Aldrich (UK) |
| | ethoxylate | ethoxylate | | |
| 153. | Laureth-8 | AEC | 3055-98-9 | A & E Connock |
| | | Laureth-8 | | (Perfumery & |
| | | | | Cosmetics) Ltd. |
| 154. | Oleth-8 | Emalex 508 | 27040-03-5 | Nihon Emulsion |
| | | | | Company, Ltd. |
| 155. | Laureth-7 | Alfonic | 3055-97-8 | Sasol North |
| | | 1216CO-7 | | America, Inc. |
| | | Ethoxylate | | |
| 156. | Steareth-7 | Polyoxyethylene | 66146-84-7 | Sigma Aldrich |
| | | (7) stearyl ether | | |
| 157. | Deceth-6 | Alfonic 1012- | 5168-89-8 | Sasol North |
| | | 6.0 Ethoxylate | | America, Inc. |
| 158. | Steareth-6 | Emalex 606 | 2420-29-3 | Nihon Emulsion |
| | | | | Company, Ltd. |
| 159. | Hexaethylene glycol | Hexaethylene glycol | 3055-96-7 | Sigma-Aldrich (UK) |
| | monododecyl ether | monododecyl ether | | |
| 160. | Hexaethylene glycol | Hexaethylene glycol | 5168-91-2 | Sigma-Aldrich (UK) |
| | monohexadecyl ether | monohexadecyl ether | | |
| 161. | Beheneth-5 | Nikkol BB-5 | 136207-49-3 | Nikko Chemicals |
| | | | | Co., Ltd. |
| 162. | Myreth-5 | Isoxal 12 | 92669-01-7 | Vevy Europe SpA |
| 163. | Steareth-5 | Jeecol SA-5 | 71093-13-5 | Jeen |
| | | | | International |
| | | | | Corporation |
| 164. | Ceteth-5 | Emalex 105 | 4478-97-1 | Nihon Emulsion |
| | | | | Company, Ltd. |
| 165. | Oleth-5 | Brij ® O5 | 5353-27-5 | Croda, Inc. |
| 166. | Laureth-5 | Safol ® 23E5 | 3055-95-6 | Sasol North |
| | | Ethoxylate | | America, Inc. |
| 167. | Steareth-4 | Jeecol SA-4 | 59970-10-4 | Jeen |
| | | | | International |
| | | | | Corporation |
| 168. | Laureth-4 | Brij ® L4 | 5274-68-0 | Croda, Inc. |
| 169. | Myreth-4 | Homulgator 920 G | 39034-24-7 | Grau Aromatics |
| | | | | GmbH & |
| | | | | Company KG |
| 170. | Ceteth-4 | Procol CA-4 | 5274-63-5 | Protameen |
| | | | | Chemicals |
| 171. | Oleth-4 | Chemal OA-4 | 5353-26-4 | Chemax, Inc. |
| 172. | Oleth-4 | Chemal OA-4 | 103622-85-1 | Chemax, Inc. |
| 173. | Polyimide-1 | Aquaflex ™ | 497926-97-3 | Chemwill |
| | | XL-30 | | |
| 174. | Polymethoxy Bicyclic | Caswell No. | 56709-13-8 | Angene Chemical |
| | Oxazolidine | 494CA | | |
| 175. | Hydroxymethyl | Zoldine ™ ZT | 6542-37-6 | Angus Chemical |
| | Dioxoazabicyclooctane | | | Company |
| 176. | Dihydro-7a- | 5-Ethyl-1-aza- | 7747-35-5 | Sigma Aldrich (UK) |
| | ethyloxazolo[3,4- | 3,7-dioxabicy- | | |
| | c]oxazole | clo[3.3.0]octane | | |
| 177. | Dibenzylidene | Disorbene ® | 32647-67-9 | Roquette |
| | Sorbitol | | | America, Inc. |
| 178. | Dimethyldibenzylidene | Millad ® 3988 | 135861-56-2 | Milliken |
| | Sorbitol | | | Chemicals |
| 179. | Laureth-2 | Alfonic | 3055-93-4 | Sasol North |
| | | 1216CO-2 | | America, Inc. |
| | | Ethoxylate | | |
| 180. | 2-(2- | Piperonyl | 51-03-6 | Sigma-Aldrich (UK) |
| | Butoxyethoxy)ethyl | Butoxide | | |
| | (6-propylpiperonyl) | | | |
| | ether | | | |
| 181. | Menthone | Frescolat ® | 63187-91-7 | Symrise |
| | Glycerin Acetal | MGA | | |
| 182. | Propylene Glycol | Mackaderm | 68332-79-6 | Rhodia Inc. |
| | Caprylate | PGC | | |
| 183. | Diethoxynonadiene | SBB016951 | 67674-36-6 | Ambinter |
| 184. | Menthoxypropanediol | Coolact ® 10 | 87061-04-9 | Takasago |
| | Alcohols | | | International |
| | | | | Corporation |
| 185. | 2-Diphenylmethoxy- | Diphenhydramine | 147-24-0 | Sigma-Aldrich (UK) |
| | N,N- | HCl | | |
| | dimethylethylamine | | | |
| | hydrochloride | | | |
| 186. | 3-((2-ethyl- | — | 70445-33-9 | — |
| | hexyl)oxy)propane- | | | |
| | 1,2-diol | | | |
| 187. | 3-((2-propyl- | — | — | — |
| | heptyl)oxy)propane- | | | |
| | 1,2-diol | | | |
| 188. | 1-amino-3-((2- | — | 99509-00-9 | — |
| | ethylhexyl)oxy)propan- | | | |
| | 2-ol | | | |
| 189. | 1-(1-Methyl-2- | Di(propylene | 29911-27-1 | Sigma Aldrich (UK) |
| | propoxyethoxy)- | glycol) propyl | | |
| | 2-propanol | ether | | |

As described further herein. The compounds, as described above in Tables 1(a) and 1(b), act as a substantially non-odorous fragrance modulator of fragrance materials of the present invention. Without wishing to be bound by theory, it is believed that the substantially non-odorous fragrance modulators associate to fragrance materials to allow for high or overdosed wt % values of the fragrance materials (e.g., greater than 30 wt %) in a composition to which the coating contacts will allow the overdosed fragrance materials to drive the perceived character of the fragrance, while mitigating or eliminating a perceived harshness of the composition by the user. The non-odorous fragrance modulators can also be used in materials that do not include overdosed fragrance materials.

The composition including the substantially non-odorous fragrance modulator according to the present invention, can include a volatile solvent, a non-volatile solvent, or a mixture thereof. The solvent can be organic or aqueous. Where present, the volatile solvent can be present in the amount of from about 20 wt % to about 99 wt % relative to the total weight of the composition, about 30 wt % to about 80 wt %, about 55 wt % to about 75 wt %, or less than, equal to, or greater than about 20 wt %, 25, 30, 35, 40, 45, 50, 55, 60, 65, 70, 75, 80, 85, 90, 95, or about 99 wt %, and wherein the solvent is a branch or unbranched C₁ to C₁₀ alkyl, akenyl or alkynyl group having at least one alcohol moiety, preferably ethanol, or isopropanol, or other alcohols (e.g., methanol, propanol, isopropanol, butanol, and mixtures thereof) commonly found in commercial fine fragrance products.

Accordingly, ethanol may be present in any of the compositions of the present invention, and more specifically, it will form from about 5 wt % to about 95 wt %, or even from about 10 wt % to about 80 wt %, 25 wt % to about 75 wt % of the composition, or combinations thereof, relative to the total weight of the composition. Alternatively, ethanol may be present in an amount of from about 10 wt % or 25 wt % to about 75 wt % or 80 wt %, relative to the total weight of the composition. The ethanol useful in the present invention may be any acceptable quality of ethanol, compatible and safe for the specific intended use of the composition such as, for example, topical applications of fine fragrance or cosmetic compositions.

The composition may comprise a non-volatile solvent or a mixture of non-volatile solvents. Non-limiting examples of non-volatile solvents include benzyl benzoate, diethyl phthalate, isopropyl myristate, propylene glycol, dipropylene glycol, triethyl citrate, and mixtures thereof. Where non-volatile solvents are present, introduced either with the perfume materials or separately, then for the purposes of calculating the proportion of fragrance component having a vapor pressure of less than 0.001 Torr (0.000133 kPa) at 25° C. the total fragrance components does not include non-volatile solvents. Where non-volatile solvents are present, introduced either with the perfume materials or separately, then for the purposes of calculating the total level of fragrance component this does not include non-volatile solvents. In addition, if present with cyclic oligosacchrides, the non-volatile solvent may be included at a weight ratio of the non-volatile solvent to the cyclic oligosaccharide of less than 1:1, less than 1:2, less than 1:10, or less than 1:100.

In some examples (e.g., those including a volatile solvent), water may be present in any of the compositions of the present invention, and more specifically, it may not exceed about 95 wt % relative to the total weight of the composition, about 90 wt % or less, about 85 wt % or less, about 80 wt % or less, about 75 wt % or less, about 70 wt % or less, about 65 wt % or less, about 60 wt % or less, about 55 wt % or less, about 50 wt % or less, about 45 wt % or less, about 40 wt % or less, about 35 wt % or less, about 30 wt % or less, about 20 wt % or less, about 10 wt %, or less than, equal to, or greater than about 95 wt %, 90, 85, 80, 75, 70, 65, 60, 55, 50, 45, 40, 30, 35, 30, 25, 20, 15, 10, or 5 wt %. Alternatively, water may be present in an amount of from about 5 wt % or about 95 wt % When the composition is a cosmetic composition the level of water should not be so high that the product becomes cloudy thus negatively impacting the product aesthetics. It is understood that the amount of water present in the composition may be from the water present in the volatile solvent (e.g., ethanol) used in the composition, as the case may be. In some examples, the coating composition contains only substantially non-odorous components.

The coating compositions described herein can be used in conjunction with a fragrance composition. The fragrance composition can include one or more fragrance components that can be characterized by their respective volatilities. For example, fragrance components can be low volatility, moderate volatility, or high volatility fragrance components. According to various embodiments, a fragrance composition can be overdosed with one or more of the fragrance components such that fragrance composition can be considered to be a bottom-heavy fragrance, a middle-heavy fragrance, a top-heavy fragrance composition, or a composition where fragrance components are evenly distributed.

Each group of materials can be associated with various perceptions by a panel of experts or professional evaluators or individual experts or professional evaluators. While not so limited, a high volatile fragrance may be associated with a citrus character; a moderate volatile fragrance may be associated with a spicy character; and a low volatile fragrance may be associated with a woody character. Each group of fragrance materials can include synthetic materials or natural materials. The volatility of the fragrance materials can be in reference to an individual fragrance material. Alternatively, in cases where a combination of materials produce a fragrance the volatility may be in reference to that aggregation.

With respect to a fragrance composition, the fragrance component can be present in an amount of from about 0.04 wt % to 30 wt %, 1 wt % to about 30 wt %, about 5 wt % to about 30 wt %, or less than, equal to, or greater than about 0.04 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or about 30 wt % relative to the composition. Additional components in the fragrance composition can include a carrier such as the organic, inorganic, or aqueous carriers described herein with respect to the coating composition.

Additionally, according to various embodiments, it is possible for the fragrance composition to include any one or more of the modulators described herein with respect to the coating composition. Where present in the coating composition, the modulator can be present in an amount of from about 0.1 wt % to about 20 wt %, about 0.5 wt % to about 20 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 wt % relative to the coating composition.

As described herein, the “fragrance materials” have been classified as low, moderate or high volatile fragrance materials according to their volatility by their vapor pressure. This method of classifying fragrance materials by their vapor pressure avoids the problem of different classifications for the same fragrance material according to the traditional approach that relies on their subjective characteristic character. In the case that the fragrance materials are a natural oil, extract or absolute, which comprises a mixture of several compounds, the vapor pressure of the complete oil should be treated a mixture of the individual perfume raw material components using the reference program cited above. The individual components and their level, in any given natural oil or extract, can be determined by direct injection of the oil into a GC-MS column for analysis as known by one skilled in the art. In the scenario that the fragrance materials are a proprietary specialty accord, so called ‘bases’, the vapor pressure, using the reference program cited above, should preferably be obtained from the supplier. However, it is understood by one skilled in the art that they can physically analyze the composition of a full fragrance oil available commercially to identity the fragrance raw materials and their levels using standard GC-MS techniques. This would be irrespective of whether they had been added to the fragrance oil as individual chemicals, as components of naturals or from proprietary bases. Although proprietary bases and naturals are included in our examples, when analyzing a commercially available fragrance via GC-MS one could simply identify the components of the base or natural oil as part of the overall fragrance mixture and their levels, without being able to identify which proprietary base or natural oil the fragrance had come from.

Low Volatile Fragrance Materials

The fragrance component comprises at least one low volatile fragrance material. Individual low volatile fragrance materials or aggregate low volatile fragrance materials are those having a vapor pressure less than 0.001 Torr (0.000133 kPa) at 25° C. According to some examples, the composition can include at least 3 low volatile fragrance materials, or at least 4 low volatile fragrance materials, or at least 5 low volatile fragrance materials, or at least 7 low volatile fragrance materials. The amount of the low volatile fragrance material present in the fragrance component can vary depending on the specific application. In some embodiments, the low volatile fragrance component can be in a range of from about 0 wt % to about 100 wt % of the fragrance component, about 10 wt % to about 90 wt %, about 20 wt % to about 80 wt %, about 30 wt % to about 70 wt %, or about 40 wt % to about 60 wt %. The exact wt % of the low volatile fragrance component can vary greatly based on the fragrance's construction, for example, in a bottom-heavy embodiment, the low volatile fragrance material can be greater than about 30 wt % of the fragrance component, greater than about 40 wt %, greater than about 50 wt %, greater than about 60 wt %, about 31 wt % to about 60 wt %, about 40 wt % to about 50 wt %, or less than, equal to, or greater than about 30 wt %, 31, 35, 40, 45, 50, 55, 60, 65, 70, or 75 wt %. In embodiments that are top- or middle-heavy the low volatile fragrances can be can be present in an amount ranging from about 0 wt % to about 30 wt % of the fragrance component, about 1 wt % to about 30 wt %, about 1 wt % to about 29 wt %, or less than, equal to, or greater than about 0 wt %, 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.25, 27, 27.5, 28, 28.5, 29, 29.5, or about 30 wt %. In still further embodiments, the low volatile fragrance material can be in an amount ranging from about 30 wt % to about 40 wt % of the fragrance component, about 32 wt % to about 35 wt %, less than, equal to, or greater than about 30 wt % 31, 32, 33, 34, 35, 36, 37, 38, 39, or 40 wt %. In some embodiments, the low-volatile fragrance material can be the only fragrance material present. Relative to any other fragrance materials, the low volatile fragrance component can be present in a ratio ranging from about 20:1 to about 1:20, about 10:1 to about 1:10, about 5:1 to about 1:5, or about 1:1.

In some embodiments, the low volatile fragrance materials can be present as a component of the coating composition as well. In these embodiments, the low volatile fragrance material can be present in a range of from about 0.01 wt % to about 50 wt %.

If there are more than one low volatile fragrance materials, then the ranges provided hereinabove cover the total of all the low volatile fragrance materials. Examples of suitable low volatile fragrances materials are provided in Table 2A and 2B below.

Preferably, the low volatile fragrance material is selected from at least 1 material, or at least 2 materials, or at least 3 materials, or at least 5 materials, at least 7, at least 8, at least 10, or at least 12 low volatile fragrance materials as disclosed in Table 2A. Natural fragrance materials or oils having an aggregate vapor pressure less than 0.001 Torr (0.000133 kPa) at 25° C. are provided in Table 2B. Low Volatile Natural Oils.

TABLE 2A
Low Volatile Fragrance Materials

| | | | | Vapor Pressure |
|---|---|---|---|---|
| No. | CAS Number | IUPAC Name | Common Name** | (Torr at 25° C.)* |
| 1. | 1211-29-6 | Cyclopentaneacetic acid, | Methyl | 0.00096500 |
| | | 3-oxo-2-(2Z)-2-penten-1- | jasmonate | |
| | | yl-, methyl ester, | | |
| | | (1R,2R)- | | |
| 2. | 28219-60-5 | 2-Buten-1-ol, 2-methyl- | Hindinol | 0.00096100 |
| | | 4-(2,2,3-trimethyl-3- | | |
| | | cyclopenten-1-yl)- | | |
| 3. | 93-08-3 | Ethanone, 1-(2- | Methyl beta- | 0.00095700 |
| | | naphthalenyl)- | naphthyl | |
| | | | ketone | |
| 4 | 67633-95-8 | 3-Decanone, 1-hydroxy- | Methyl | 0.00095100 |
| | | | Lavender | |
| | | | Ketone | |
| 5. | 198404-98-7 | Cyclopropanemethanol, | Javanol ® | 0.00090200 |
| | | 1-methyl-2-[(1,2,2- | | |
| | | trimethylbicyclo[3.1.0]hex- | | |
| | | 3-yl)methyl]- | | |
| 6. | 121-32-4 | Benzaldehyde, 3-ethoxy- | Ethyl vanillin | 0.00088400 |
| | | 4-hydroxy- | | |
| 7. | 72403-67-9 | 3-Cyclohexene-1- | Myraldylacetate | 0.00087900 |
| | | methanol, 4-(4-methyl-3- | | |
| | | penten-1-yl)-, 1-acetate | | |
| 8. | 28940-11-6 | 2H-1,5-Benzodioxepin- | Calone | 0.00083100 |
| | | 3(4H)-one, 7-methyl- | | |
| 9. | 139504-68-0 | 2-Butanol, 1-[[2-(1,1- | Amber core | 0.00080300 |
| | | dimethylethyl)cyclohexyl]oxy]- | | |
| 10. | 502847-01-0 | Spiro[5.5]undec- | Spiro[5.5]undec- | 0.00073100 |
| | | 8-en-1-one, | 8-en-1-one, | |
| | | 2,2,7,9-tetramethyl- | 2,2,7,9-tetramethyl- | |
| 11. | 2570-03-8 | Cyclopentaneacetic acid, | trans-Hedione | 0.00071000 |
| | | 3-oxo-2-pentyl-, methyl | | |
| | | ester, (1R,2R)-rel- | | |
| 12. | 24851-98-7 | Cyclopentaneacetic acid, | Methyl | 0.00071000 |
| | | 3-oxo-2-pentyl-, methyl | dihydrojasmonate | |
| | | ester | or alternatives ¹ | |
| 13. | 101-86-0 | Octanal, 2- | Hexyl | 0.00069700 |
| | | (phenylmethylene)- | cinnamic | |
| | | | aldehyde | |
| 14. | 365411-50-3 | Indeno[4,5-d]-1,3-dioxin, | Nebulone | 0.00069200 |
| | | 4,4a,5,6,7,8,9,9b- | | |
| | | octahydro-7,7,8,9,9- | | |
| | | pentamethyl- | | |
| 15. | 37172-53-5 | Cyclopentanecarboxylic | Dihydro Iso | 0.00067500 |
| | | acid, 2-hexyl-3-oxo-, | Jasmonate | |
| | | methyl ester | | |
| 16. | 65113-99-7 | 3-Cyclopentene-1- | Sandalore ® | 0.00062500 |
| | | butanol, α,β,3,2,2,3- | | |
| | | pentamethyl- | | |
| 17. | 68133-79-9 | Cyclopentanone, 2-(3,7- | Apritone | 0.00062000 |
| | | dimethyl-2,6-octadien-1- | | |
| | | yl)- | | |
| 18. | 7212-44-4 | 1,6,10-Dodecatrien-3-ol, | Nerolidol | 0.00061600 |
| | | 3,7,11-trimethyl- | | |
| 19. | 53243-59-7 | 2-Pentenenitrile, 3- | Citronitril | 0.00061500 |
| | | methyl-5-phenyl-, (2Z)- | | |
| 20. | 134123-93-6 | Benzenepropanenitrile, | Fleuranil | 0.00057600 |
| | | 4-ethyl-α,α-dimethyl- | | |
| 21. | 77-53-2 | 1H-3a,7-Methanoazulen- | Cedrol Crude | 0.00056900 |
| | | 6-ol, octahydro-3,6,8,8- | | |
| | | tetramethyl-, | | |
| | | (3R,3aS,6R,7R,8aS)- | | |
| 22. | 68155-66-8 | Ethanone, 1- | Iso Gamma | 0.00056500 |
| | | (1,2,3,5,6,7,8,8a- | Super | |
| | | octahydro-2,3,8,8- | | |
| | | tetramethyl-2- | | |
| | | naphthalenyl)- | | |
| 23. | 54464-57-2 | Ethanone, 1- | Iso-E Super ® | 0.00053800 |
| | | (1,2,3,4,5,6,7,8- | | |
| | | octahydro-2,3,8,8- | | |
| | | tetramethyl-2- | | |
| | | naphthalenyl)- | | |
| 24. | 774-55-0 | Ethanone, 1-(5,6,7,8- | Florantone | 0.00053000 |
| | | tetrahydro-2- | | |
| | | naphthalenyl)- | | |
| 25. | 141-92-4 | 2-Octanol, 8,8- | Hydroxycitronellal | 0.00052000 |
| | | dimethoxy-2,6- | Dimethyl | |
| | | dimethyl- | Acetal | |
| 26. | 20665-85-4 | Propanoic acid, 2- | Vanillin | 0.00051200 |
| | | methyl-, 4-formyl-2- | isobutyrate | |
| | | methoxyphenyl ester | | |
| 27. | 79-78-7 | 1,6-Heptadien-3-one, 1- | Hexalon | 0.00049800 |
| | | (2,6,6-trimethyl-2- | | |
| | | cyclohexen-1-yl)- | | |
| 28. | 6259-76-3 | Benzoic acid, 2-hydroxy-, | Hexyl | 0.00049100 |
| | | hexyl ester | Salicylate | |
| 29. | 93-99-2 | Benzoic acid, phenyl | Phenyl | 0.00047900 |
| | | ester | Benzoate | |
| 30. | 153859-23-5 | Cyclohexanepropanol, | Norlimbanol | 0.00046900 |
| | | 2,2,6-trimethyl-α-propyl-, | | |
| | | (1R,6S)- | | |
| 31. | 70788-30-6 | Cyclohexanepropanol, | Timberol/ | 0.00046900 |
| | | 2,2,6-trimethyl-α-propyl- | Norlimbanol | |
| 32. | 68555-58-8 | Benzoic acid, 2-hydroxy-, | Prenyl | 0.00045700 |
| | | 3-methyl-2-buten-1-yl | Salicylate | |
| | | ester | | |
| 33. | 950919-28-5 | 2H-1,5-Benzodioxepin- | Cascalone | 0.00045500 |
| | | 3(4H)-one, 7-(1- | | |
| | | methylethyl)- | | |
| 34. | 30168-23-1 | Butanal, 4-(octahydro- | Dupical | 0.00044100 |
| | | 4,7-methano-5H-inden-5- | | |
| | | ylidene)- | | |
| 35. | 1222-05-5 | Cyclopenta[g]-2-, | Galaxolide ® | 0.00041400 |
| | | benzopyran, 1,3,4,7,8- | | |
| | | hexahydro-4,6,6,7,8,8- | | |
| | | hexamethyl- | | |
| 36. | 4602-84-0 | 2,6,10-Dodecatrien-1-ol, | Farnesol | 0.00037000 |
| | | 3,7,11-trimethyl- | | |
| 37. | 95962-14-4 | Cyclopentanone, 2-[2-(4- | Nectaryl | 0.00036700 |
| | | methyl-3-cyclohexen-1- | | |
| | | yl)propyl]- | | |
| 38. | 4674-50-4 | 2(3H)-Naphthalenone, | Nootkatone | 0.00035800 |
| | | 4,4a,5,6,7,8-hexahydro- | | |
| | | 4,4a-dimethyl-6-(1- | | |
| | | methylethenyl)-, | | |
| | | (4R,4aS,6R)- | | |
| 39. | 3487-99-8 | 2-Propenoic acid, 3- | Amyl | 0.00035200 |
| | | phenyl-, pentyl ester | Cinnamate | |
| 40. | 10522-41-5 | 2-hydroxy-2-phenylethyl | Styrolyl | 0.00033900 |
| | | acetate | Acetate | |
| 41. | 118-71-8 | 4H-Pyran-4-one, 3- | Maltol | 0.00033700 |
| | | hydroxy-2-methyl- | | |
| 42. | 128119-70-0 | 1-Propanol, 2-methyl-3- | Bornafix | 0.00033400 |
| | | [(1,7,7-tri- | | |
| | | methylbicyclo[2.2.1]hept- | | |
| | | 2-yl)oxy]- | | |
| 43. | 103614-86-4 | 1-Naphthalenol, | Octalynol | 0.00033200 |
| | | 1,2,3,4,4a,5,8,8a- | | |
| | | octahydro-2,2,6,8- | | |
| | | tetramethyl- | | |
| 44. | 7785-33-3 | 2-Butenoic acid, 2- | Geranyl | 0.00033200 |
| | | methyl-, (2E)-3,7- | Tiglate | |
| | | dimethyl-2,6-octadien-1- | | |
| | | yl ester, (2E)- | | |
| 45. | 117933-89-8 | 1,3-Dioxane, 2-(2,4- | Karanal | 0.00033100 |
| | | dimethyl-3-cyclohexen- | | |
| | | 1-yl)-5-methyl-5-(1- | | |
| | | methylpropyl)- | | |
| 46. | 629-92-5 | Nonadecane | Nonadecane | 0.00032500 |
| 47. | 67801-20-1 | 4-Penten-2-ol, 3-methyl- | Ebanol | 0.00028100 |
| | | 5-(2,2,3-trimethyl-3- | | |
| | | cyclopenten-1-yl)- | | |
| 48. | 65416-14-0 | Propanoic acid, 2- | Maltol | 0.00028000 |
| | | methyl-, 2-methyl-4-oxo- | Isobutyrate | |
| | | 4H-pyran-3-yl ester | | |
| 49. | 28219-61-6 | 2-Buten-1-ol, 2-ethyl-4- | Laevo | 0.00028000 |
| | | (2,2,3-trimethyl-3- | Trisandol | |
| | | cyclopenten-1-yl)- | | |
| 50. | 5986-55-0 | 1,6-Methanonaphthalen- | Healingwood | 0.00027800 |
| | | 1(2H)-ol, octahydro- | | |
| | | 4,8a,9,9-tetramethyl-, | | |
| | | (1R,4S,4aS,6R,8aS)- | | |
| 51. | 195251-91-3 | 2H-1,5-Benzodioxepin- | Transluzone | 0.00026500 |
| | | 3(4H)-one, 7-(1,1- | | |
| | | dimethylethyl)- | | |
| 52. | 3100-36-5 | 8- Cyclohexadecen-1-one | Cyclohexadecenone | 0.00025300 |
| 53. | 65405-77-8 | Benzoic acid, 2-hydroxy-, | cis-3-Hexenyl | 0.00024600 |
| | | (3Z)-3-hexen-1-yl ester | salicylate | |
| 54. | 4940-11-8 | 4H-Pyran-4-one, 2-ethyl- | Ethyl Maltol | 0.00022800 |
| | | 3-hydroxy- | | |
| 55. | 541-91-3 | Cyclopentadecanone, 3- | Muskone | 0.00017600 |
| | | methyl- | | |
| 56. | 118-58-1 | Benzoic acid, 2-hydroxy-, | Benzyl | 0.00017500 |
| | | phenylmethyl ester | salicylate | |
| 57. | 81783-01-9 | 6,8-Nonadien-3-one, | Labienoxime | 0.00017300 |
| | | 2,4,4,7-tetramethyl-, | | |
| | | oxime | | |
| 58. | 25485-88-5 | Benzoic acid, 2-hydroxy-, | Cyclohexyl | 0.00017300 |
| | | cyclohexyl ester | Salicylate | |
| 59. | 91-87-2 | Benzene, [2- | Amyl | 0.00016300 |
| | | (dimethoxymethyl)-1- | Cinnamic | |
| | | hepten-1-yl]- | Aldehyde | |
| | | | Dimethyl | |
| | | | Acetal | |
| 60. | 104864-90-6 | 3-Cyclopentene-1- | Firsantol | 0.00016000 |
| | | butanol, β,2,2,3- | | |
| | | tetramethyl-δ-methylene- | | |
| 61. | 224031-70-3 | 4-Penten-1-one, 1- | Spirogalbanone | 0.00015300 |
| | | spiro[4.5]dec-7-en-7-yl- | | |
| 62. | 134-28-1 | 5-Azulenemethanol, | Guaiyl | 0.00013400 |
| | | 1,2,3,4,5,6,7,8- | Acetate | |
| | | octahydro-α,α,3,8- | | |
| | | tetramethyl-, 5-acetate, | | |
| | | (3S,5R,8S)- | | |
| 63. | 236391-76-7 | Acetic acid, 2-(1- | Romandolide ® | 0.00012400 |
| | | oxopropoxy)-, 1-(3,3- | | |
| | | dimethylcyclohexy)ethyl | | |
| | | ester | | |
| 64. | 115-71-9 | 2-Penten-1-ol, 5- | cis-alpha- | 0.00011800 |
| | | [(1R,3R,6S)-2,3- | Santalol | |
| | | dimethyltricy- | | |
| | | clo[2.2.1.02,6]hept- | | |
| | | 3-yl]-2-methyl-, (2Z)- | | |
| 65. | 107898-54-4 | 4-Penten-2-ol, 3,3- | Polysantol ® | 0.00011700 |
| | | dimethyl-5-(2,2,3- | | |
| | | trimethyl-3-cyclopenten- | | |
| | | 1-yl)- | | |
| 66. | 69486-14-2 | 5,8-Methano-2H-1- | Florex ® | 0.00011000 |
| | | benzopyran-2-one, 6- | | |
| | | ethylideneoctahydro- | | |
| 67. | 84697-09-6 | Heptanal, 2-[(4- | Acalea | 0.00010100 |
| | | methylphenyl)methylene]- | | |
| 68. | 14595-54-1 | 4-Cyclopentadecen-1- | Exaltenone | 0.00009640 |
| | | one, (4Z)- | | |
| 69. | 32388-55-9 | Ethanone, 1- | Vertofix ® | 0.00008490 |
| | | [(3R,3aR,7R,8aS)- | | |
| | | 2,3,4,7,8,8a-hexahydro- | | |
| | | 3,6,8,8-tetramethyl-1H- | | |
| | | 3a,7-methanoazulen-5- | | |
| | | yl]- | | |
| 70. | 131812-67-4 | 1,3-Dioxolane, 2,4- | Okoumal ® | 0.00007600 |
| | | dimethyl-2-(5,6,7,8- | | |
| | | tetrahydro-5,5,8,8- | | |
| | | tetramethyl-2- | | |
| | | naphthalenyl)- | | |
| 71. | 106-02-5 | Oxacyclohexadecan-2- | Exaltolide ® | 0.00006430 |
| | | one | | |
| 72. | 141773-73-1 | 1-Propanol, 2-[1-(3,3- | Helvetolide ® | 0.00005790 |
| | | dimethylcy- | | |
| | | clohexyl)ethoxy]-2- | | |
| | | methyl-, 1-propanoate | | |
| 73. | 63314-79-4 | 5-Cyclopentadecen-1- | Delta | 0.00005650 |
| | | one, 3-methyl- | Muscenone | |
| 74. | 77-42-9 | 2-Penten-1-ol, 2-methyl- | cis-beta- | 0.00004810 |
| | | 5-[(1S,2R,4R)-2-methyl-3- | Santalol | |
| | | methylenebicyclo[2.2.1]hept- | | |
| | | 2-yl]-, (2Z)- | | |
| 75. | 362467-67-2 | 2H-1,5-Benzodioxepin- | Azurone | 0.00004770 |
| | | 3(4H)-one, 7-(3- | | |
| | | methylbutyl)- | | |
| 76. | 28371-99-5 | Ethanone, 1-(2,6,10- | Trimofix O | 0.00004580 |
| | | trimethyl-2,5,9- | | |
| | | cyclododecatrien-1-yl)- | | |
| 77. | 16223-63-5 | 1H-3a,6- | Khusimol | 0.00004400 |
| | | Methanoazulene-3- | | |
| | | methanol, octahydro-7,7- | | |
| | | dimethyl-8-methylene-, | | |
| | | (3S,3aR,6R,8aS)- | | |
| 78. | 10461-98-0 | Benzeneacetonitrile, α- | Peonile | 0.00004290 |
| | | cyclohexylidene- | | |
| 79. | 90-17-5 | Benzenemethanol, α- | Rosacetol | 0.00004240 |
| | | (trichloromethyl)-, 1- | | |
| | | acetate | | |
| 80. | 50607-64-2 | Benzoic acid, 2-[(2- | Mevantraal | 0.00004070 |
| | | methylpentylidene)amino]-, | | |
| | | methyl ester | | |
| 81. | 29895-73-6 | 5-Hydroxy-2-benzyl-1,3- | Acetal CD | 0.00004050 |
| | | dioxane | | |
| 82. | 94-47-3 | Benzoic acid, 2- | Phenyl Ethyl | 0.00003480 |
| | | phenylethyl ester | Benzoate | |
| 83. | 3100-36-5 | Cyclohexadec-8-en-1- | Globanone ® | 0.00003310 |
| | | one | | |
| 84. | 37609-25-9 | 5-Cyclohexadecen-1-One | Ambretone | 0.00003310 |
| 85. | 66072-32-0 | Cyclohexanol, 4-(1,7,7-tri- | Iso Bornyl | 0.00003010 |
| | | methylbicyclo[2.2.1]hept- | Cyclohexanol | |
| | | 2-yl)- | | |
| 86. | 31906-04-4 | 3-Cyclohexene-1- | Lyral ® | 0.00002940 |
| | | carboxaldehyde, 4-(4- | | |
| | | hydroxy-4- | | |
| | | methylpentyl)- | | |
| 87. | 21145-77-7 | Ethanone, 1-(5,6,7,8- | Musk Plus | 0.00002860 |
| | | tetrahydro-3,5,5,6,8,8- | | |
| | | hexamethyl-2- | | |
| | | naphthalenyl)- | | |
| 88. | 21145-77-7 | Ethanone, 1-(5,6,7,8- | Fixolide | 0.00002860 |
| | | tetrahydro-3,5,5,6,8,8- | | |
| | | hexamethyl-2- | | |
| | | naphthalenyl)- | | |
| 89. | 22442-01-9 | 2-Cyclopentadecen-1- | Muscenone | 0.00002770 |
| | | one, 3-methyl- | | |
| 90. | 109-29-5 | Oxacycloheptadecan-2- | Silvanone Ci | 0.00002600 |
| | | one | | |
| 91. | 101-94-0 | Benzeneacetic acid, 4- | Para Cresyl | 0.00002330 |
| | | methylphenyl ester | Phenyl Acetate | |
| 92. | 102-20-5 | Benzeneacetic acid, 2- | Phenyl Ethyl | 0.00002300 |
| | | phenylethyl ester | Phenyl Acetate | |
| 93. | 118562-73-5 | Cyclododecaneethanol, | Hydroxyambran | 0.00001800 |
| | | β-methyl- | | |
| 94. | 103-41-3 | 2-Propenoic acid, 3- | Benzyl | 0.00001050 |
| | | phenyl-, phenylmethyl | Cinnamate | |
| | | ester | | |
| 95. | 4707-47-5 | Benzoic acid, 2,4- | Veramoss/ | 0.00001050 |
| | | dihydroxy-3,6-dimethyl-, | LRG201/ | |
| | | methyl ester | Evernyl | |
| 96. | 183551-83-9 | Naphtho[2,1-b]furan- | Myrrhone | 0.00000977 |
| | | 6(7H)-one, 8,9-dihydro- | | |
| | | 1,5,8-trimethyl-, (8R)- | | |
| 97. | 102-17-0 | Benzeneacetic acid, (4- | Para Anisyl | 0.00000813 |
| | | methoxyphenyl)methyl | Phenyl Acetate | |
| | | ester | | |
| 98. | 120-11-6 | Benzene, 2-methoxy-1- | Benzyl Iso | 0.00000676 |
| | | (phenylmethoxy)-4-(1- | Eugenol | |
| | | propen-1-yl)- | | |
| 99. | 102-22-7 | Benzeneacetic acid, (2E)- | Geranyl | 0.00000645 |
| | | 3,7-dimethyl-2,6- | Phenylacetate | |
| | | octadien-1-yl ester | | |
| 100. | 111879-80-2 | Oxacyclohexadec-12-en- | Habanolide | 0.00000431 |
| | | 2-one, (12E)- | 100% | |
| 101. | 87-22-9 | Benzoic acid, 2-hydroxy-, | Phenyl Ethyl | 0.00000299 |
| | | 2-phenylethyl ester | Salicylate | |
| 102. | 78-37-5 | 2-Propenoic acid, 3- | Linalyl | 0.00000174 |
| | | phenyl -, 1-ethenyl-1,5- | Cinnamate | |
| | | dimethyl-4-hexen-1-yl | | |
| | | ester | | |
| 103. | 28645-51-4 | Oxacycloheptadec-10-en- | Ambrettolide | 0.00000139 |
| | | 2-one | | |
| 104. | 123-69-3 | Oxacycloheptadec-8-en- | Ambrettolide | 0.00000136 |
| | | 2-one, (8Z)- | | |
| 105. | 3391-83-1 | 1,7- | Musk RI | 0.00000057 |
| | | Dioxacycloheptadecan-8- | | |
| | | one | | |
| 106. | 68527-79-7 | 7-Octen-2-ol, 8-(1H- | Indolene | 0.000000445 |
| | | indol-1-yl)-2,6-dimethyl- | | |
| 107. | 89-43-0 | Methyl 2-[(7-hydroxy- | Aurantinol | 0.0000000100 |
| | | 3,7-dimethyloc- | | |
| | | tylidene)amino]benzoate | | |
| 108. | 54982-83-1 | 1,4- | Zenolide | 0.00000000834 |
| | | Dioxacyclohexadecane- | | |
| | | 5,16-dione | | |
| 109. | 105-95-3 | 1,4- | Ethylene | 0.00000000313 |
| | | Dioxacycloheptadecane- | Brassylate | |
| | | 5,17-dione | | |
| 110. | 3681-73-0 | Hexadecanoic acid, (2E)- | Hexarose | 0.00000000300 |
| | | 3,7-dimethyl-2,6- | | |
| | | octadien-1-yl ester | | |
| 111. | 4159-29-9 | Phenol, 4-[3- | Coniferyl | 0.00000000170 |
| | | (benzoyloxy)-1-propen- | benzoate | |
| | | 1-yl]-2-methoxy- | | |
| 112. | 144761-91-1 | Benzoic acid, 2-[(1- | Trifone DIPG | 0.00000000093 |
| | | hydroxy-3- | | |
| | | phenylbutyl)amino]-, | | |
| | | methyl ester | | |

¹ Non-limiting examples of alternative qualities from various suppliers can be purchased under the following tradenames: Kharismal ® Super (IFF), Kharismal ® (IFF), Hedione ® (Firmenich), Hedione ® HC (Firmenich), Paradisone (Firmenich), Cepionate (Zenon), Super cepionate (Zenon), Claigeon ® (Zenon).
*Vapor Pressures are acquired as described in the Test Methods Section.
**Origin: The low volatile fragrance materials may be obtained from one or more of the following companies: Firmenich (Geneva, Switzerland), Symrise AG (Holzminden, Germany), Givaudan (Argenteuil, France), IFF (Hazlet, New Jersey), Bedoukian (Danbury, Connecticut), Sigma Aldrich (St. Louis, Missouri), Millennium Specialty Chemicals (Olympia Fields, Illinois), Polarone International (Jersey City, New Jersey), and Aroma & Flavor Specialties (Danbury, Connecticut).

TABLE 2B
Low Volatile Natural Oils.

| No. | Natural oil | Supplier |
|---|---|---|
| 1. | Beeswax Absolute | Robertet |
| 2. | Cedarwood Sawdust SFE | Firmenich |
| 3. | Cedarwood Oil Rect | Firmenich |
| 4. | Cedarwood Texas Light | H. Reynaud & Fils |
| 5. | Ciste Absolute | IFF |
| 6. | Cocoa Colorless Oil | Robertet |
| 7. | Cypriol Coeur Essence | Robertet |
| 8.. | Guaiacwood Oil | Global Essence Inc |
| 9. | Incense Wood Natural | Robertet |
| 10. | Orris CO2 Extract | Mane |
| 11. | Patchouli Oil | IFF |
| 12. | Tolu Baume Res | Robertet |
| 13. | Vanilla Absolute | Robertet |
| 14. | Vanilla CO2 Absolute | Robertet |
| 15. | Vetivert Oil | IFF |
| 16. | Vetyvert Acetate | Robertet |

Suppliers:
Firmenish, Geneva, Switzerland
Global Essence Inc, New Jersey, USA
H. Reynaud & Fils, Montbrun-les-Bains, France
IFF, Hazlet, New Jersey, USA
Mane, Le Bar-sur-Loup, France
Robertet, Grasse, France

Exemplary low volatile fragrance materials selected from the group of Tables 2A or 2B Low Volatile Fragrance Materials are preferred. However, it is understood by one skilled in the art that other low volatile fragrance materials, not recited in Tables 2A or 2B, would also fall within the scope of the present invention, so long as they have a vapor pressure less than 0.001 Torr (0.000133 kPa) at 25° C.

Moderate Volatile Fragrance Materials

The fragrance component includes at least one moderate volatile fragrance material or aggregate of volatile fragrance materials having a vapor pressure in the range of 0.1 Torr to 0.001 Torr (0.0133 kPa to 0.000133 kPa) at 25° C. In some examples, the composition according to the present disclosure can include at least 3 moderate volatile fragrance materials, or at least 5 moderate volatile fragrance materials, or at least 7 moderate volatile fragrance materials. Compositions of the present invention can include any suitable amount of the moderate volatile fragrance materials. For example, in some embodiments, the moderate volatile fragrance component can be in a range of from about 0 wt % to about 100 wt % of the fragrance component, about 10 wt % to about 90 wt %, about 20 wt % to about 80 wt %, about 30 wt % to about 70 wt %, or about 40 wt % to about 60 wt %. The exact wt % of the moderate volatile fragrance component can vary greatly based on the fragrance's construction. The exact wt % of various moderate volatile fragrance components can depend on the fragrance's construction. For example, in some constructions, high levels of the moderate volatile fragrance materials present in an amount of from about 30 wt % to about 75 wt %, about 35 wt % to about 60 wt %, or less than equal to, or greater than about 30 wt %, 35, 40, 45, 50, 55, 60, 65, 70, or 75 wt % of the fragrance component. If there are more than one moderate volatile fragrance materials, then the ranges provided hereinabove cover the total of all of the moderate volatile fragrance materials. Suitable examples of moderate volatile fragrances materials are provided in Table 3A and 3B below. Alternatively, in further embodiments, the moderate volatile fragrance material can be present in an amount ranging from about 0 wt % to about 30 wt % of the fragrance component, about 1 wt % to about 30 wt %, about 1 wt % to about 29 wt %, or less than, equal to, or greater than about 0 wt %, 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.25, 27, 27.5, 28, 28.5, 29, 29.5, or about 30 wt %. In still further embodiments, the moderate volatile fragrance material can be in an amount ranging from about 30 wt % to about 40 wt % of the fragrance component, about 32 wt % to about 35 wt %, less than, equal to, or greater than about 30 wt % 31, 32, 33, 34, 35, 36, 37, 38, 39, or 40 wt %. In some embodiments, the moderate volatile fragrance material can be the only fragrance material present. Relative to any other fragrance materials, the moderate volatile fragrance component can be present in a ratio ranging from about 20:1 to about 1:20, about 10:1 to about 1:10, about 5:1 to about 1:5, or about 1:1.

In some embodiments, the moderate volatile fragrance materials can be present as a component of the coating composition as well. In these embodiments, the moderate volatile fragrance material can be present in a range of from about 0.01 wt % to about 50 wt %.

Preferable examples of moderate volatile fragrance materials having a vapor pressure in the range of 0.1 Torr to 0.001 Torr (0.0133 kPa to 0.000133 kPa) at 25° C. are provided in Table 3 Moderate Volatile Fragrance Materials. Preferably, the moderate volatile fragrance material is selected from at least 1 material, or at least 2 materials, or at least 3 materials, or at least 5 materials, or at least 7 moderate volatile fragrance materials as disclosed in Table 3A. Natural fragrance materials or oils having an aggregate vapor pressure between 0.1 Torr to 0.001 Torr (0.0133 kPa to 0.000133 kPa) at 25° C. are provided in Table 3B. Moderate Volatile Natural Oils.

TABLE 3A
Moderate Volatile Fragrance Materials

| | | | | Vaporat Pressure |
|---|---|---|---|---|
| No. | CAS Number | IUPAC Name | Common Name** | (Torr at 25° C.)* |
| 1. | 24168-70-5 | Pyrazine, 2- | Methoxyisobutyl- | 0.09950000 |
| | | methoxy-3-(1- | pyrazine | |
| | | methylpropyl)- | | |
| 2. | 89-79-2 | Cyclohexanol, 5- | Iso-Pulegol | 0.09930000 |
| | | methyl-2-(1- | | |
| | | methylethenyl)-, | | |
| | | (1R,2S,5R)- | | |
| 3. | 112-12-9 | 2-Undecanone | Methyl Nonyl | 0.09780000 |
| | | | Ketone | |
| 4. | 103-05-9 | Benzenepropanol, | Phenyl Ethyl | 0.09770000 |
| | | α,α-dimethyl- | Dimethyl | |
| | | | Carbinol | |
| 5. | 125-12-2 | Bicyclo[2.2.1]heptan- | Iso Bornyl | 0.09590000 |
| | | 2-ol, 1,7,7- | Acetate | |
| | | trimethyl-, 2-acetate, | | |
| | | (1R,2R,4R)-rel- | | |
| 6. | 78-70-6 | 1,6-Octadien-3-ol, | Linalool | 0.09050000 |
| | | 3,7-dimethyl- | | |
| 7. | 101-97-3 | Benzeneacetic acid, | Ethyl Phenyl | 0.08970000 |
| | | ethyl ester | Acetate | |
| 8. | 100-86-7 | Benzeneethanol, | Dimethyl Benzyl | 0.08880000 |
| | | α,α-dimethyl- | Carbinol | |
| 9. | 188570-78-7 | Cyclopropanecarboxylic | Montaverdi | 0.08640000 |
| | | acid, (3Z)-3- | | |
| | | hexen-1-yl ester | | |
| 10. | 67634-25-7 | 3-Cyclohexene-1- | Floralate | 0.08500000 |
| | | methanol, 3,5- | | |
| | | dimethyl-, 1-acetate | | |
| 11. | 112-44-7 | Undecanal | Undecyl | 0.08320000 |
| | | | Aldehyde | |
| 12. | 32669-00-4 | Ethanone, 1-(3- | Tanaisone ® | 0.08150000 |
| | | cycloocten-1-yl)- | | |
| 13. | 98-53-3 | Cyclohexanone, 4- | Patchi | 0.07780000 |
| | | (1,1-dimethylethyl)- | | |
| 14. | 35854-86-5 | 6-Nonen-1-ol, (6Z)- | cis-6-None-1-ol | 0.07770000 |
| 15. | 5331-14-6 | Benzene, (2- | Butyl phenethyl | 0.07760000 |
| | | butoxyethyl)- | ether | |
| 16. | 80-57-9 | Bicyclo[3.1.1]hept- | Verbenone | 0.07730000 |
| | | 3-en-2-one, 4,6,6- | | |
| | | trimethyl- | | |
| 17. | 22471-55-2 | Cyclohexanecarboxylic | Thesaron | 0.07670000 |
| | | acid, 2,2,6- | | |
| | | trimethyl-, ethyl | | |
| | | ester, (1R,6S)-rel- | | |
| 18. | 60-12-8 | Benzeneethanol | Phenethyl alcohol | 0.07410000 |
| 19. | 106-26-3 | 2,6-Octadienal, 3,7- | Neral | 0.07120000 |
| | | dimethyl-, (2Z)- | | |
| 20. | 5392-40-5 | 2,6-Octadienal, 3,7- | Citral | 0.07120000 |
| | | dimethyl- | | |
| 21. | 89-48-5 | Cyclohexanol, 5- | Menthyl Acetate | 0.07070000 |
| | | methyl-2-(1- | | |
| | | methylethyl)-, 1- | | |
| | | acetate, (1R,2S,5R)- | | |
| | | rel- | | |
| 22. | 119-36-8 | Benzoic acid, 2- | Methyl salicylate | 0.07000000 |
| | | hydroxy-, methyl | | |
| | | ester | | |
| 23. | 104-46-1 | Benzene, 1- | Anethol | 0.06870000 |
| | | methoxy-4-(1E)-1- | | |
| | | propen-1-yl- | | |
| 24. | 7549-37-3 | 2,6-Octadiene, 1,1- | Citral Dimethyl | 0.06780000 |
| | | dimethoxy-3,7- | Acetal | |
| | | dimethyl- | | |
| 25. | 25225-08-5 | Cyclohexanemethanol | Aphermate | 0.06780000 |
| | | α,3,3-trimethyl-, | | |
| | | 1-formate | | |
| 26. | 3913-81-3 | 2-Decenal, (2E)- | 2-Decene-1-al | 0.06740000 |
| 27. | 15373-31-6 | 3-Cyclopentene-1- | Cantryl ® | 0.06700000 |
| | | acetonitrile, 2,2,3- | | |
| | | trimethyl- | | |
| 28. | 6485-40-1 | 2-Cyclohexen-1-one, | Laevo carvone | 0.06560000 |
| | | 2-methyl-5-(1- | | |
| | | methylethenyl)-, | | |
| | | (5R)- | | |
| 29. | 16587-71-6 | Cyclohexanone, 4- | Orivone | 0.06490000 |
| | | (1,1- | | |
| | | dimethylpropyl)- | | |
| 30. | 62406-73-9 | 6,10- | Opalal CI | 0.06290000 |
| | | Dioxaspiro[4.5]decane, | | |
| | | 8,8-dimethyl-7- | | |
| | | (1-methylethyl)- | | |
| 31. | 3720-16-9 | 2-Cyclohexen-1-one, | Livescone | 0.06270000 |
| | | 3-methyl-5-propyl- | | |
| 32. | 13816-33-6 | Benzonitrile, 4-(1- | Cumin Nitrile | 0.06230000 |
| | | methylethyl)- | | |
| 33. | 67019-89-0 | 2,6-Nonadienenitrile | Violet Nitrile | 0.06200000 |
| 34. | 53398-85-9 | Butanoic acid, 2- | cis-3-Hexenyl | 0.06130000 |
| | | methyl-, (3Z)-3- | Alpha Methyl | |
| | | hexen-1-yl ester | Butyrate | |
| 35. | 208041-98-9 | n/a | Jasmonitrile | 0.05920000 |
| 36. | 16510-27-3 | Benzene, 1- | Toscanol | 0.05870000 |
| | | (cyclopropylmethyl)- | | |
| | | 4-methoxy- | | |
| 37. | 111-80-8 | 2-Nonynoic acid, | Methyl Octine | 0.05680000 |
| | | methyl ester | Carbonate | |
| 38. | 103-45-7 | Acetic acid, 2- | Phenyl Ethyl | 0.05640000 |
| | | phenylethyl ester | Acetate | |
| 39. | 2550-26-7 | 2-Butanone, 4- | Benzyl Acetone | 0.05570000 |
| | | phenyl- | | |
| 40. | 13491-79-7 | Cyclohexanol, 2- | Verdol | 0.05430000 |
| | | (1,1-dimethylethyl)- | | |
| 41. | 7786-44-9 | 2,6-Nonadien-1-ol | 2,6-Nonadien-1-ol | 0.05370000 |
| 42. | 103-28-6 | Propanoic acid, 2- | Benzyl Iso | 0.05130000 |
| | | methyl-, | Butyrate | |
| | | phenylmethyl ester | | |
| 43. | 104-62-1 | Formic acid, 2- | Phenyl Ethyl | 0.05050000 |
| | | phenylethyl ester | Formate | |
| 44. | 28462-85-3 | Bicyclo[2.2.1]heptan- | Humus Ether | 0.04870000 |
| | | 2-ol, 1,2,3,3- | | |
| | | tetramethyl-, | | |
| | | (1R,2R,4S)-rel- | | |
| 45. | 122-03-2 | Benzaldehyde, 4-(1- | Cuminic | 0.04820000 |
| | | methylethyl)- | Aldehyde | |
| 46. | 358331-95-0 | 2,5-Octadien-4-one, | Pomarose | 0.04810000 |
| | | 5,6,7-trimethyl-, | | |
| | | (2E)- | | |
| 47. | 562-74-3 | 3-Cyclohexen-1-ol, | Terpinenol-4 | 0.04780000 |
| | | 4-methyl-1-(1- | | |
| | | methylethyl)- | | |
| 48. | 68527-77-5 | 3-Cyclohexene-1- | Isocyclogeraniol | 0.04640000 |
| | | methanol, 2,4,6- | | |
| | | trimethyl- | | |
| 49. | 35852-46-1 | Pentanoic acid, (3Z)- | Cis-3-Hexenyl | 0.04580000 |
| | | 3-hexen-1-yl ester | Valerate | |
| 50. | 2756-56-1 | Bicyclo[2.2.1]heptan- | Iso Bornyl | 0.04540000 |
| | | 2-ol, 1,7,7- | Propionate | |
| | | trimethyl-, 2- | | |
| | | propanoate, | | |
| | | (1R,2R,4R)-rel- | | |
| 51. | 14374-92-6 | Benzene, 1-methyl- | Verdoracine | 0.04460000 |
| | | 4-(1-methylethyl)-2- | | |
| | | (1-propen-1-yl)- | | |
| 52. | 6784-13-0 | 3-Cyclohexene-1- | Limonenal | 0.04380000 |
| | | propanal, β,4- | | |
| | | dimethyl- | | |
| 53. | 8000-41-7 | 2-(4-methyl-1- | Alpha Terpineol | 0.04320000 |
| | | cyclohex-3- | | |
| | | enyl)propan-2-ol | | |
| 54. | 41884-28-0 | 1-Hexanol, 5- | Tetrahydro | 0.04230000 |
| | | methyl-2-(1- | Lavandulol | |
| | | methylethyl)-, (2R)- | | |
| 55. | 22457-23-4 | 3-Heptanone, 5- | Stemone ® | 0.04140000 |
| | | methyl-, oxime | | |
| 56. | 104-50-7 | 2(3H)-Furanone, 5- | Gamma | 0.04080000 |
| | | butyldihydro- | Octalactone | |
| 57. | 143-08-8 | 1-Nonanol | Nonyl Alcohol | 0.04070000 |
| 58. | 3613-30-7 | Octanal, 7-methoxy- | Methoxycitronellal | 0.04020000 |
| | | 3,7-dimethyl- | | |
| 59. | 67634-00-8 | Acetic acid, 2-(3- | Ally Amyl | 0.04000000 |
| | | methylbutoxy)-, 2- | Glycolate | |
| | | propen-1-yl ester | | |
| 60. | 464-45-9 | Bicyclo[2.2.1]heptan- | 1-Borneol | 0.03980000 |
| | | 2-ol, 1,7,7-trimethyl-, | | |
| | | (1S,2R,4S)- | | |
| 61. | 124-76-5 | Bicyclo[2.2.1]heptan- | 1.7.7-Trimethyl- | 0.03980000 |
| | | 2-ol, 1,7,7- | Bicyclo-1.2.2- | |
| | | trimethyl-, | Heptanol-2 | |
| | | (1R,2R,4R)-rel- | | |
| 62. | 67874-72-0 | Cyclohexanol, 2- | Coniferan | 0.03980000 |
| | | (1,1- | | |
| | | dimethylpropyl)-, 1- | | |
| | | acetate | | |
| 63. | 80-26-2 | 3-Cyclohexene-1- | Terpinyl Acetate | 0.03920000 |
| | | methanol, α,α,4- | | |
| | | trimethyl-, 1-acetate | | |
| 64. | 498-81-7 | Cyclohexanemethanol, | Dihydro | 0.03920000 |
| | | α,α,4-trimethyl- | Terpineol | |
| 65. | 112-45-8 | 10-Undecenal | Undecylenic | 0.03900000 |
| | | | aldehyde | |
| 66. | 35044-57-6 | 2,4-Cyclohexadiene- | Ethyl Safranate | 0.03880000 |
| | | 1-carboxylic acid, | | |
| | | 2,6,6-trimethyl-, | | |
| | | ethyl ester | | |
| 67. | 106-21-8 | 1-Octanol, 3,7- | Dimethyl Octanol | 0.03860000 |
| | | dimethyl- | | |
| 68. | 84560-00-9 | Cyclopentanol, 2- | Cyclopentol | 0.03790000 |
| | | pentyl- | | |
| 69. | 82461-14-1 | Furan, tetrahydro- | Rhubafuran ® | 0.03780000 |
| | | 2,4-dimethyl-4- | | |
| | | phenyl- | | |
| 70. | 56011-02-0 | Benzene, [2-(3- | Phenyl Ethyl | 0.03690000 |
| | | methylbutoxy)ethyl]- | Isoamyl Ether | |
| 71. | 103-37-7 | Butanoic acid, | Benzyl Butyrate | 0.03660000 |
| | | phenylmethyl ester | | |
| 72. | 6378-65-0 | Hexyl hexanoate | Hexyl hexanoate | 0.03490000 |
| 73. | 118-61-6 | Benzoic acid, 2- | Ethyl salicylate | 0.03480000 |
| | | hydroxy-, ethyl ester | | |
| 74. | 98-52-2 | Cyclohexanol, 4- | Patchon | 0.03480000 |
| | | (1,1-dimethylethyl)- | | |
| 75. | 115-99-1 | 1,6-Octadien-3-ol, | Linalyl Formate | 0.03440000 |
| | | 3,7-dimethyl-, 3- | | |
| | | formate | | |
| 76. | 112-54-9 | Dodecanal | Lauric Aldehyde | 0.03440000 |
| 77. | 53046-97-2 | 3,6-Nonadien-1-ol, | 3,6-Nonadien-1-ol | 0.03360000 |
| | | (3Z,6Z)- | | |
| 78. | 76649-25-7 | 3,6-Nonadien-1-ol | 3,6-Nonadien-1-ol | 0.03360000 |
| 79. | 141-25-3 | 3,7-Dimethyloct-6- | Rhodinol | 0.03290000 |
| | | en-1-ol | | |
| 80. | 1975-78-6 | Decanenitrile | Decanonitrile | 0.03250000 |
| 81. | 2216-51-5 | Cyclohexanol, 5- | L-Menthol | 0.03230000 |
| | | methyl-2-(1- | | |
| | | methylethyl)-, | | |
| | | (1R,2S,5R)- | | |
| 82. | 3658-77-3 | 4-hydroxy-2,5- | Pineapple Ketone | 0.03200000 |
| | | dimethylfuran-3-one | | |
| 83. | 103-93-5 | Propanoic acid, 2- | Para Cresyl iso- | 0.03120000 |
| | | methyl-, 4- | Butyrate | |
| | | methylphenyl ester | | |
| 84. | 24717-86-0 | Propanoic acid, 2- | Abierate | 0.03110000 |
| | | methyl-, (1R,2S,4R)- | | |
| | | 1,7,7-trimethylbicy- | | |
| | | clo[2.2.1]hept- | | |
| | | 2-yl ester, rel- | | |
| 85. | 67845-46-9 | Acetaldehyde, 2-(4- | Aldehyde XI | 0.03090000 |
| | | methylphenoxy)- | | |
| 86. | 67883-79-8 | 2-Butenoic acid, 2- | Cis-3-Hexenyl | 0.03060000 |
| | | methyl-, (3Z)-3- | Tiglate | |
| | | hexen-1-yl ester, | | |
| | | (2E)- | | |
| 87. | 33885-51-7 | Bicyclo[3.1.1]hept- | Pino | 0.03040000 |
| | | 2-ene-2-propanal, | Acetaldehyde | |
| | | 6,6-dimethyl- | | |
| 88. | 105-85-1 | 6-Octen-1-ol, 3,7- | Citronellyl | 0.03000000 |
| | | dimethyl-, 1-formate | Formate | |
| 89. | 70214-77-6 | 2-Nonanol, 6,8- | Nonadyl | 0.03010000 |
| | | dimethyl- | | |
| 90. | 215231-33-7 | Cyclohexanol, 1- | Rossitol | 0.02990000 |
| | | methyl-3-(2- | | |
| | | methylpropyl)- | | |
| 91. | 120-72-9 | 1H-Indole | Indole | 0.02980000 |
| 92. | 2463-77-6 | 2-Undecenal | 2-Undecene-1-al | 0.02970000 |
| 93. | 675-09-2 | 2H-Pyran-2-one, | Levistamel | 0.02940000 |
| | | 4,6-dimethyl- | | |
| 94. | 98-55-5 | 3-Cyclohexene-1- | Alpha-Terpineol | 0.02830000 |
| | | methanol, α,α,4- | | |
| | | trimethyl- | | |
| 95. | 81786-73-4 | 3-Hepten-2-one, | Koavone | 0.02750000 |
| | | 3,4,5,6,6- | | |
| | | pentamethyl-, (3Z- | | |
| 96. | 122-97-4 | Benzenepropanol | Phenyl Propyl | 0.02710000 |
| | | | Alcohol | |
| 97. | 39212-23-2 | 2(3H)-Furanone, 5- | Methyl | 0.02700000 |
| | | butyldihydro-4- | Octalactone | |
| | | methyl- | | |
| 98. | 53767-93-4 | 7-Octen-2-ol, 2,6- | Dihydro Terpinyl | 0.02690000 |
| | | dimethyl-, 2-acetate | Acetate | |
| 99. | 35044-59-8 | 1,3-Cyclohexadiene- | Ethyl Safranate | 0.02660000 |
| | | 1-carboxylic acid, | | |
| | | 2,6,6-trimethyl-, | | |
| | | ethyl ester | | |
| 100. | 104-55-2 | 2-Propenal, 3- | Cinnamic | 0.02650000 |
| | | phenyl- | Aldehyde | |
| 101. | 144-39-8 | 1,6-Octadien-3-ol, | Linalyl | 0.02630000 |
| | | 3,7-dimethyl-, 3- | Propionate | |
| | | propanoate | | |
| 102. | 61931-80-4 | 1,6-Nonadien-3-ol, | 3,7-Dimethyl-1,6- | 0.02630000 |
| | | 3,7-dimethyl-, 3- | nonadien-3-yl | |
| | | acetate | acetate | |
| 103. | 102-13-6 | Benzeneacetic acid, | Iso Butyl | 0.02630000 |
| | | 2-methylpropyl ester | Phenylacetate | |
| 104. | 65443-14-3 | Cyclopentanone, | Veloutone | 0.02610000 |
| | | 2,2,5-trimethyl-5- | | |
| | | pentyl- | | |
| 105. | 141-12-8 | 2,6-Octadien-1-ol, | Neryl Acetate | 0.02560000 |
| | | 3,7-dimethyl-, 1- | | |
| | | acetate, (2Z)- | | |
| 106. | 105-87-3 | 2,6-Octadien-1-ol, | Geranyl acetate | 0.02560000 |
| | | 3,7-dimethyl-, 1- | | |
| | | acetate, (2E)- | | |
| 107. | 68141-17-3 | Undecane, 1,1- | Methyl Nonyl | 0.02550000 |
| | | dimethoxy-2- | Acetaldehyde | |
| | | methyl- | Dimethyl Acetal | |
| 108. | 2206-94-2 | Benzenemethanol, | Indocolore | 0.02550000 |
| | | α-methylene-, 1- | | |
| | | acetate | | |
| 109. | 10528-67-3 | Cyclohexanepropanol, | Cyclohexylmagnol | 0.02550000 |
| | | α-methyl- | | |
| 110. | 123-11-5 | Benzaldehyde, 4- | Anisic Aldehyde | 0.02490000 |
| | | methoxy- | | |
| 111. | 57576-09-7 | Cyclohexanol, 5- | Iso Pulegol | 0.02480000 |
| | | methyl-2-(1- | Acetate | |
| | | methylethenyl)-, 1- | | |
| | | acetate, (1R,2S,5R)- | | |
| 112. | 51566-62-2 | 6-Octenenitrile, 3,7- | Citronellyl Nitrile | 0.02470000 |
| | | dimethyl- | | |
| 113. | 60335-71-9 | 2H-Pyran, 3,6- | Rosyrane Super | 0.02470000 |
| | | dihydro-4-methyl-2- | | |
| | | phenyl- | | |
| 114. | 30385-25-2 | 6-Octen-2-ol, 2,6- | Dihydromyrcenol | 0.02440000 |
| | | dimethyl- | | |
| 115. | 101-84-8 | Benzene, 1,1′- | Diphenyl Oxide | 0.02230000 |
| | | oxybis- | | |
| 116. | 136-60-7 | Benzoic acid, butyl | Butyl Benzoate | 0.02170000 |
| | | ester | | |
| 117. | 93939-86-7 | 5,8-Methano-2H-1- | Rhuboflor | 0.02120000 |
| | | benzopyran, 6- | | |
| | | ethylideneoctahydro- | | |
| 118. | 83926-73-2 | Cyclohexanepropanol, | Coranol | 0.02100000 |
| | | α,α-dimethyl- | | |
| 119. | 125109-85-5 | Benzenepropanal, β- | Florhydral | 0.02070000 |
| | | methyl-3-(1- | | |
| | | methylethyl)- | | |
| 120. | 104-21-2 | Benzenemethanol, | Anisyl Acetate | 0.02050000 |
| | | 4-methoxy-, | | |
| | | 1-acetate | | |
| 121. | 1365-19-1 | 2-Furanmethanol, 5- | Linalool Oxide | 0.02050000 |
| | | ethenyltetrahydro- | | |
| | | α,α,5-trimethyl- | | |
| 122. | 137-03-1 | Cyclopentanone, 2- | Frutalone | 0.02040000 |
| | | heptyl- | | |
| 123. | 2563-07-7 | Phenol, 2-ethoxy-4- | Ultravanil | 0.02030000 |
| | | methyl- | | |
| 124. | 1128-08-1 | 2-Cyclopenten-1- | Dihydrojasmone | 0.02020000 |
| | | one, 3-methyl-2- | | |
| | | pentyl- | | |
| 125. | 7493-57-4 | Benzene, [2-(1- | Acetaldehyde | 0.01990000 |
| | | propoxyethoxy)ethyl]- | | |
| 126. | 141-25-3 | 7-Octen-1-ol, 3,7- | Rhodinol | 0.01970000 |
| | | dimethyl- | | |
| 127. | 216970-21-7 | Bicyclo[4.3.1]decane, | 3-Methoxy-7,7- | 0.01960000 |
| | | 3-methoxy-7,7- | dimethyl-10- | |
| | | dimethyl-10- | methylenebicyclo | |
| | | methylene- | [4.3.1.]decane | |
| 128. | 319002-92-1 | Propanoic acid, 2- | Sclareolate ® | 0.01960000 |
| | | (1,1- | | |
| | | dimethylpropoxy)-, | | |
| | | propyl ester, (2S)- | | |
| 129. | 85-91-6 | Benzoic acid, 2- | Dimethyl | 0.01930000 |
| | | (methylamino)-, | anthranilate | |
| | | methyl | | |
| 130. | 13828-37-0 | Cyclohexanemethanol, | Mayol | 0.01920000 |
| | | 4-(1- | | |
| | | methylethyl)-, cis- | | |
| 131. | 26330-65-4 | (E)-6-ethyl-3- | Super Muguet | 0.01850000 |
| | | methyloct-6-en-1-ol | | |
| 132. | 7540-51-4 | 6-Octen-1-ol, 3,7- | L-Citronellol | 0.01830000 |
| | | dimethyl-, (3S)- | | |
| 133. | 106-22-9 | 6-Octen-1-ol, 3,7- | Citronellol | 0.01830000 |
| | | dimethyl- | | |
| 134. | 543-39-5 | 7-Octen-2-ol, 2- | Myrcenol | 0.01820000 |
| | | methyl-6-methylene- | | |
| 135. | 7775-00-0 | Benzenepropanal, 4- | Cyclemax | 0.01820000 |
| | | (1-methylethyl)- | | |
| 136. | 18479-54-4 | 4,6-Octadien-3-ol, | Muguol | 0.01800000 |
| | | 3,7-dimethyl- | | |
| 137. | 29214-60-6 | Octanoic acid, 2- | Gelsone | 0.01790000 |
| | | acetyl-, ethyl ester | | |
| 138. | 1209-61-6 | 5-Oxatricy- | Tobacarol | 0.01730000 |
| | | clo[8.2.0.04,6]dodecane, | | |
| | | 4,9,12,12- | | |
| | | tetramethyl- | | |
| 139. | 57934-97-1 | 2-Cyclohexene-1- | Givescone | 0.01710000 |
| | | carboxylic acid, 2- | | |
| | | ethyl-6,6-dimethyl-, | | |
| | | ethyl ester | | |
| 140. | 14901-07-6 | 3-Buten-2-one, 4- | Beta-Ionone | 0.01690000 |
| | | (2,6,6-trimethyl-1- | | |
| | | cyclohexen-1-yl)-, | | |
| | | (3E)- | | |
| 141. | 64001-15-6 | 4,7-Methano-1H- | Dihydro Cyclacet | 0.01630000 |
| | | inden-5-ol, | | |
| | | octahydro-, 5-acetate | | |
| 142. | 95-41-0 | 2-Cyclopenten-1- | Iso Jasmone T | 0.01600000 |
| | | one, 2-hexyl- | | |
| 143. | 134-20-3 | Benzoic acid, 2- | Methyl | 0.01580000 |
| | | amino-, methyl ester | Anthranilate | |
| 144. | 100-06-1 | Ethanone, 1-(4- | Para Methoxy | 0.01550000 |
| | | methoxyphenyl)- | Acetophenone | |
| 145. | 105-86-2 | 2,6-Octadien-1-ol, | Geranyl Formate | 0.01540000 |
| | | 3,7-dimethyl-, 1- | | |
| | | formate, (2E)- | | |
| 146. | 154171-77-4 | Spiro[1,3-dioxolane- | Ysamber K ® | 0.01470000 |
| | | 2,8′(5′H)-[2H- | | |
| | | 2,4a]methanonaphthalene], | | |
| | | hexahydro- | | |
| | | 1′,1′,5′,5′- | | |
| | | tetramethyl, | | |
| | | (2′S,4′aS,8′aS)- | | |
| | | (9CI) | | |
| 147. | 154171-76-3 | Spiro[1,3-dioxolane- | Ysamber | 0.01470000 |
| | | 2,8′(5′H)-[2H- | | |
| | | 2,4a]methanonaphthalene], | | |
| 148. | 127-41-3 | 3-Buten-2-one, 4- | Alpha-Ionone | 0.01440000 |
| | | (2,6,6-trimethyl-2- | | |
| | | cyclohexen-1-yl)-, | | |
| | | (3E)- | | |
| 149. | 151-05-3 | Benzeneethanol, | Dimethyl Benzyl | 0.01390000 |
| | | α,α-dimethyl-, 1- | Carbinyl Acetate | |
| | | acetate | | |
| 150. | 2500-83-6 | 4,7-Methano-1H- | Flor Acetate | 0.01370000 |
| | | inden-5-ol, | | |
| | | 3a,4,5,6,7,7a- | | |
| | | hexahydro-, 5- | | |
| | | acetate | | |
| 151. | 150-84-5 | 6-Octen-1-ol, 3,7- | Citronellyl | 0.01370000 |
| | | dimethyl-, 1-acetate | acetate | |
| 152. | 30310-41-9 | 2H-Pyran, | Pelargene | 0.01350000 |
| | | tetrahydro-2-methyl- | | |
| | | 4-methylene-6- | | |
| | | phenyl- | | |
| 153. | 68845-00-1 | Bicyclo[3.3.1]nonane, | Boisiris | 0.01350000 |
| | | 2-ethoxy-2,6,6- | | |
| | | trimethyl-9- | | |
| | | methylene- | | |
| 154. | 106-24-1 | 2,6-Octadien-1-ol, | Geraniol | 0.01330000 |
| | | 3,7-dimethyl-, (2E)- | | |
| 155. | 106-25-2 | 2,6-Octadien-1-ol, | Nerol | 0.01330000 |
| | | 3,7-dimethyl-, (2Z)- | | |
| 156. | 75975-83-6 | Bicyclo[7.2.0]undec- | Vetyvenal | 0.01280000 |
| | | 4-ene, 4,11,11- | | |
| | | trimethyl-8- | | |
| | | methylene-, | | |
| | | (1R,4E,9S)- | | |
| 157. | 19870-74-7 | 1H-3a,7- | Cedryl methyl | 0.01280000 |
| | | Methanoazulene, | ether | |
| | | octahydro-6- | | |
| | | methoxy-3,6,8,8- | | |
| | | tetramethyl-, | | |
| | | (3R,3aS,6S,7R,8aS)- | | |
| 158. | 87-44-5 | Bicyclo[7.2.0]undec- | Caryophyllene | 0.01280000 |
| | | 4-ene, 4,11,11- | Extra | |
| | | trimethyl-8- | | |
| | | methylene-, | | |
| | | (1R,4E,9S)- | | |
| 159. | 54440-17-4 | 1H-Inden-1-one, | Safraleine | 0.01260000 |
| | | 2,3-dihydro-2,3,3- | | |
| | | trimethyl- | | |
| 160. | 110-98-5 | 2-Propanol, 1,1′- | Dipropylene | 0.01250000 |
| | | oxybis- | Glycol | |
| 161. | 41890-92-0 | 2-Octanol, 7- | Osyrol ® | 0.01250000 |
| | | methoxy-3,7- | | |
| | | dimethyl- | | |
| 162. | 71077-31-1 | 4,9-Decadienal, 4,8- | Floral Super | 0.01230000 |
| | | dimethyl- | | |
| 163. | 65-85-0 | Benzoic Acid | Benzoic Acid | 0.01220000 |
| 164. | 61444-38-0 | 3-Hexenoic acid, | cis-3-hexenyl-cis- | 0.01220000 |
| | | (3Z)-3-hexen-1-yl | 3-hexenoate | |
| | | ester, (3Z)- | | |
| 165. | 116044-44-1 | Bicyclo[2.2.1]hept- | Herbanate | 0.01210000 |
| | | 5-ene-2-carboxylic | | |
| | | acid, 3-(1- | | |
| 166. | 104-54-1 | 2-Propen-1-ol, 3- | Cinnamic | 0.01170000 |
| | | phenyl- | Alcohol | |
| 167. | 78-35-3 | Propanoic acid, 2- | Linalyl | 0.01170000 |
| | | methyl-, 1-ethenyl- | Isobutyrate | |
| | | 1,5-dimethyl-4- | | |
| | | hexen-1-yl ester | | |
| 168. | 23495-12-7 | Ethanol, 2-phenoxy-, | Phenoxy Ethyl | 0.01300000 |
| | | 1-propanoate | Propionate | |
| 169. | 103-26-4 | 2-Propenoic acid, 3- | Methyl | 0.01120000 |
| | | phenyl-, methyl ester | Cinnamate | |
| 170. | 67634-14-4 | Benzenepropanal, 2- | Florazon (ortho- | 0.01110000 |
| | | ethyl-α,α-dimethyl- | isomer) | |
| 171. | 5454-19-3 | Propanoic acid, | N-Decyl | 0.01100000 |
| | | decyl ester | Propionate | |
| 172. | 93-16-3 | Benzene, 1,2- | Methyl Iso | 0.01100000 |
| | | dimethoxy-4-(1- | Eugenol | |
| | | propen-1-yl)- | | |
| 173. | 81782-77-6 | 3-Decen-5-ol, 4- | 4-Methyl-3- | 0.01070000 |
| | | methyl- | decen-5-ol | |
| 174. | 67845-30-1 | Bicyclo[2.2.2]oct-5- | Maceal | 0.01060000 |
| | | ene-2- | | |
| | | carboxaldehyde, 6- | | |
| | | methyl-8-(1- | | |
| | | methylethyl)- | | |
| 175. | 97-53-0 | Phenol, 2-methoxy- | Eugenol | 0.01040000 |
| | | 4-(2-propen-1-yl)- | | |
| 176. | 120-57-0 | 1,3-Benzodioxole-5- | Heliotropin | 0.01040000 |
| | | carboxaldehyde | | |
| 177. | 93-04-9 | Naphthalene, 2- | Beta Naphthyl | 0.01040000 |
| | | methoxy- | Methyl Ether | |
| | | | Extra 99 | |
| 178. | 4826-62-4 | 2-Dodecenal | 2 Dodecene-1-al | 0.01020000 |
| 179. | 20407-84-5 | 2-Dodecenal, (2E)- | Aldehyde | 0.01020000 |
| | | | Mandarin | |
| 180. | 5462-06-6 | Benzenepropanal, 4- | Canthoxal | 0.01020000 |
| | | methoxy-α-methyl- | | |
| 181. | 94-60-0 | 1,4- | Dimethyl 1,4- | 0.01020000 |
| | | Cyclohexanedicarboxylic | cyclohexanedicarbox- | |
| | | acid, 1,4- | ylate | |
| | | dimethyl ester | | |
| 182. | 57378-68-4 | 2-Buten-1-one, 1- | delta-Damascone | 0.01020000 |
| | | (2,6,6-trimethyl-3- | | |
| | | cyclohexen-1-yl)- | | |
| 183. | 17283-81-7 | 2-Butanone, 4- | Dihydro Beta | 0.01020000 |
| | | (2,6,6-trimethyl-1- | Ionone | |
| | | cyclohexen-1-yl)- | | |
| 184. | 1885-38-7 | 2-Propenenitrile, 3- | Cinnamalva | 0.01010000 |
| | | phenyl-, (2E)- | | |
| 185. | 103-48-0 | Propanoic acid, 2- | Phenyl Ethyl Iso | 0.00994000 |
| | | methyl-, 2- | Butyrate | |
| | | phenylethyl ester | | |
| 186. | 488-10-8 | 2-Cyclopenten-1- | Cis Jasmone | 0.00982000 |
| | | one, 3-methyl-2- | | |
| | | (2Z)-2-penten-1-yl- | | |
| 187. | 7492-67-3 | Acetaldehyde, 2- | Citronellyloxy- | 0.00967000 |
| | | [(3,7-dimethyl-6- | acetaldehyde | |
| | | octen-1-yl)oxy]- | | |
| 188. | 68683-20-5 | 1-Cyclohexene-1- | Iso Bergamate | 0.00965000 |
| | | ethanol, 4-(1- | | |
| | | methylethyl)-, 1- | | |
| | | formate | | |
| 189. | 3025-30-7 | 2,4-Decadienoic | Ethyl 2,4- | 0.00954000 |
| | | acid, ethyl ester, | Decadienoate | |
| | | (2E,4Z)- | | |
| 190. | 103-54-8 | 2-Propen-1-ol, 3- | Cinnamyl Acetate | 0.00940000 |
| | | phenyl-, 1-acetate | | |
| 191. | 18127-01-0 | Benzenepropanal, 4- | Bourgeonal | 0.00934000 |
| | | (1,1-dimethylethyl)- | | |
| 192. | 3738-00-9 | Naphtho[2,1- | Ambrox ® or | 0.00934000 |
| | | b]furan, | Cetalox ® or | |
| | | dodecahydro- | Synambran | |
| | | 3a,6,6,9a- | | |
| | | tetramethyl- | | |
| 193. | 51519-65-4 | 1,4- | Tamisone | 0.00932000 |
| | | Methanonaphthalen- | | |
| | | 5(1H)-one, | | |
| | | 4,4a,7,8,8a- | | |
| | | hexahydro- | | |
| 194. | 148-05-1 | Dodecanoic acid, | Dodecalactone | 0.00931000 |
| | | 12-hydroxy-, λ- | | |
| | | lactone (6CI,7CI); | | |
| | | 1,12- | | |
| 195. | 6790-58-5 | (3aR,5aS,9aS,9bR)- | Ambronat ® or | 0.00930000 |
| | | 3a,6,6,9a- | Ambroxan ® | |
| | | tetramethyl- | | |
| | | 2,4,5,5a,7,8,9,9b- | | |
| | | octahydro-1H- | | |
| | | benzo[e][1]benzofuran | | |
| 196. | 86-26-0 | 1,1′-Biphenyl, 2- | Methyl Diphenyl | 0.00928000 |
| | | methoxy- | Ether | |
| 197. | 68738-94-3 | 2- | Cyclomyral ® | 0.00920000 |
| | | Naphthalenecarbox- | | |
| | | aldehyde, octahydro- | | |
| | | 8,8-dimethyl | | |
| 198. | 2705-87-5 | Cyclohexanepropanoic | Allyl | 0.00925000 |
| | | acid, 2-propen-1- | Cyclohexane | |
| | | yl ester | Propionate | |
| 199. | 7011-83-8 | 2(3H)-Furanone, 5- | Lactojasmone ® | 0.00885000 |
| | | hexyldihydro-5- | | |
| | | methyl- | | |
| 200. | 61792-11-8 | 2,6- | Lemonile ® | 0.00884000 |
| | | Nonadienenitrile, | | |
| | | 3,7-dimethyl- | | |
| 201. | 692-86-4 | 10-Undecenoic acid, | Ethyl | 0.00882000 |
| | | ethyl ester | Undecylenate | |
| 202. | 103-95-7 | Benzenepropanal, α- | Cymal | 0.00881000 |
| | | methyl-4-(1- | | |
| | | methylethyl)- | | |
| 203. | 13019-22-2 | 9-Decen-1-ol | Rosalva | 0.00879000 |
| 204. | 94201-19-1 | 1- | Methyl Laitone | 0.00872000 |
| | | Oxaspiro[4.5]decan- | 10% TEC | |
| | | 2-one, 8-methyl- | | |
| 205. | 104-61-0 | 2(3H)-Furanone, | γ-Nonalactone | 0.00858000 |
| | | dihydro-5-pentyl- | | |
| 206. | 706-14-9 | 2(3H)-Furanone, 5- | γ-Decalactone | 0.00852000 |
| | | hexyldihydro- | | |
| 207. | 24720-09-0 | 2-Buten-1-one, 1- | α-Damascone | 0.00830000 |
| | | (2,6,6-trimethyl-2- | | |
| | | cyclohexen-1-yl)-, | | |
| | | (2E)- | | |
| 208. | 39872-57-6 | 2-Buten-1-one, 1- | Isodamascone | 0.00830000 |
| | | (2,4,4-trimethyl-2- | | |
| | | cyclohexen-1-yl)-, | | |
| | | (2E)- | | |
| 209. | 705-86-2 | 2H-Pyran-2-one, | Decalactone | 0.00825000 |
| | | tetrahydro-6-pentyl- | | |
| 210. | 67634-15-5 | Benzenepropanal, 4- | Floralozone | 0.00808000 |
| | | ethyl-α,α-dimethyl - | | |
| 211. | 40527-42-2 | 1,3-Benzodioxole, 5- | Heliotropin | 0.00796000 |
| | | (diethoxymethyl)- | Diethyl Acetal | |
| 212. | 56973-85-4 | 4-Penten-1-one, 1- | Neobutenone α | 0.00763000 |
| | | (5,5-dimethyl-1- | | |
| | | cyclohexen-1-yl)- | | |
| 213. | 128-51-8 | Bicyclo[3.1.1]hept- | Nopyl Acetate | 0.00751000 |
| | | 2-ene-2-ethanol, 6,6- | | |
| | | dimethyl-, 2-acetate | | |
| 214. | 103-36-6 | 2-Propenoic acid, 3- | Ethyl Cinnamate | 0.00729000 |
| | | phenyl-, ethyl ester | | |
| 215. | 5182-36-5 | 1,3-Dioxane, 2,4,6- | Floropal ® | 0.00709000 |
| | | trimethyl-4-phenyl- | | |
| 216. | 42604-12-6 | Cyclododecane, | Boisambrene | 0.00686000 |
| | | (methoxymethoxy)- | | |
| 217. | 33885-52-8 | Bicyclo[3.1,1]hept- | Pinyl Iso Butyrate | 0.00685000 |
| | | 2-ene-2-propanal, | Alpha | |
| | | α,α,6,6-tetramethyl- | | |
| 218. | 92015-65-1 | 2(3H)- | Natactone | 0.00680000 |
| | | Benzofuranone, | | |
| | | hexahydro-3,6- | | |
| | | dimethyl- | | |
| 219. | 63767-86-2 | Cyclohexanemethanol | Mugetanol | 0.00678000 |
| | | α-methyl-4-(1- | | |
| | | methylethyl)- | | |
| 220. | 3288-99-1 | Benzeneacetonitrille, | Marenil CI | 0.00665000 |
| | | 4-(1,1- | | |
| | | dimethylethyl)- | | |
| 221. | 35044-68-9 | 2-Buten-1-one, 1- | beta-Damascone | 0.00655000 |
| | | (2,6,6-trimethyl-1- | | |
| | | cyclohexen-1-yl)- | | |
| 222. | 41724-19-0 | 1,4- | Plicatone | 0.00652000 |
| | | Methanonaphthalen- | | |
| | | 6(2H)-one, | | |
| | | octahydro-7-methyl | | |
| 223. | 75147-23-8 | Bicyclo[3.2,1]octan- | Buccoxime ® | 0.00647000 |
| | | 8-one, 1,5-dimethyl-, | | |
| | | oxime | | |
| 224. | 25634-93-9 | 2-Methyl-5- | Rosaphen ® | 0.00637000 |
| | | phenylpentan-1-ol | 600064 | |
| 225. | 55066-48-3 | 3-Methyl-5- | Phenyl Hexanol | 0.00637000 |
| | | phenylpentanol | | |
| 226. | 495-62-5 | Cyclohexene, 4-(1,5- | Bisabolene | 0.00630000 |
| | | dimethyl-4-hexen-1- | | |
| | | ylidene)-1-methyl- | | |
| 227. | 2785-87-7 | Phenol, 2-methoxy- | Dihydro Eugenol | 0.00624000 |
| | | 4-propyl- | | |
| 228. | 87-19-4 | Benzoic acid, 2- | Iso Butyl | 0.00613000 |
| | | hydroxy-, 2- | Salicylate | |
| | | methylpropyl ester | | |
| 229. | 4430-31-3 | 2H-1-Benzopyran-2- | Octahydro | 0.00586000 |
| | | one, octahydro- | Coumarin | |
| 230. | 38462-22-5 | Cyclohexanone, 2- | Ringonol 50 TEC | 0.00585000 |
| | | (1-mercapto-1- | | |
| | | methylethyl)-5- | | |
| | | methyl- | | |
| 231. | 77-83-8 | 2-Oxiranecarboxylic | Ethyl Methyl | 0.00571000 |
| | | acid, 3-methyl-3- | Phenyl Glycidate | |
| | | phenyl-, ethyl ester | | |
| 232. | 37677-14-8 | 3-Cyclohexene-1- | Iso Hexenyl | 0.00565000 |
| | | carboxaldehyde, 4- | Cyclohexenyl | |
| | | (4-methyl-3-penten- | Carboxaldehyde | |
| | | 1-yl)- | | |
| 233. | 103-60-6 | Propanoic acid, 2- | Phenoxy Ethyl | 0.00562000 |
| | | methyl-, 2- | iso-Butyrate | |
| | | phenoxyethyl ester | | |
| 234. | 18096-62-3 | Indeno[1,2-d]-1,3- | Indoflor ® | 0.00557000 |
| | | dioxin, 4,4a,5,9b- | | |
| | | tetrahydro- | | |
| 235. | 63500-71-0 | 2H-Pyran-4-ol, | Florosa Q/Florol | 0.00557000 |
| | | tetrahydro-4-methyl- | | |
| | | 2-(2-methylpropyl)- | | |
| 236. | 65405-84-7 | Cyclohexanebutanal, | Cetonal ® | 0.00533000 |
| | | α,2,6,6-tetramethyl- | | |
| 237. | 171102-41-3 | 4,7-Methano-1H- | Flor Acetate | 0.00530000 |
| | | inden-6-ol, | | |
| | | 3a,4,5,6,7,7a- | | |
| | | hexahydro-8,8- | | |
| | | dimethyl-, 6-acetate | | |
| 238. | 10339-55-6 | 1,6-Nonadien-3-ol, | Ethyl linalool | 0.00520000 |
| | | 3,7-dimethyl- | | |
| 239. | 23267-57-4 | 3-Buten-2-one, 4- | Ionone Epoxide | 0.00520000 |
| | | (2,2,6-trimethyl-7- | Beta | |
| | | oxabicyclo[4.1.0]hept- | | |
| | | 1-yl)- | | |
| 240. | 97-54-1 | Phenol, 2-methoxy- | Isoeugenol | 0.00519000 |
| | | 4-(1-propen-1-yl)- | | |
| 241. | 67663-01-8 | 2(3H)-Furanone, 5- | Peacholide | 0.00512000 |
| | | hexyldihydro-4- | | |
| | | methyl- | | |
| 242. | 33885-52-8 | Bicyclo[3.1.1]hept- | Pinyl Iso Butyrate | 0.00512000 |
| | | 2-ene-2-propanal, | Alpha | |
| | | α,α,6,6-tetramethyl- | | |
| 243. | 23696-85-7 | 2-Buten-1-one, 1- | Damascenone | 0.00503000 |
| | | (2,6,6-trimethyl-1,3- | | |
| | | cyclohexadien-1-yl)- | | |
| 244. | 80-71-7 | 2-Cyclopenten-1- | Maple Lactone | 0.00484000 |
| | | one, 2-hydroxy-3- | | |
| | | methyl- | | |
| 245. | 67662-96-8 | Propanoic acid, 2,2- | Pivarose Q | 0.00484000 |
| | | dimethyl-, 2- | | |
| | | phenylethyl ester | | |
| 246. | 2437-25-4 | Dodecanenitrile | Clonal | 0.00480000 |
| 247. | 141-14-0 | 6-Octen-1-ol, 3,7- | Citronellyl | 0.00469000 |
| | | dimethyl-, 1- | Propionate | |
| | | propanoate | | |
| 248. | 54992-90-4 | 3-Buten-2-one, 4- | Myrrhone | 0.00460000 |
| | | (2,2,3,6- | | |
| | | tetramethylcyclohexyl)- | | |
| 249. | 55066-49-4 | Benzenepentanal, β- | Mefranal | 0.00455000 |
| | | methyl- | | |
| 250. | 7493-74-5 | Acetic acid, 2- | Allyl Phenoxy | 0.00454000 |
| | | phenoxy-, 2-propen- | Acetate | |
| | | 1-yl ester | | |
| 251. | 80-54-6 | Benzenepropanal, 4- | Lilial ® | 0.00444000 |
| | | (1,1-dimethylethyl)- | | |
| | | α-methyl- | | |
| 252. | 86803-90-9 | 4,7-Methano-1H- | Scentenal ® | 0.00439000 |
| | | indene-2- | | |
| | | carboxaldehyde, | | |
| | | octahydro-5- | | |
| | | methoxy- | | |
| 253. | 68991-97-9 | 2-Naphtha- | Melafleur | 0.00436000 |
| | | lenecarboxaldehyde, | | |
| | | 1,2,3,4,5,6,7,8- | | |
| | | octahydro-8,8- | | |
| | | dimethyl- | | |
| 254. | 18871-14-2 | Pentitol, 1,5- | Jasmal | 0.00434000 |
| | | anhydro-2,4- | | |
| | | dideoxy-2-pentyl-, | | |
| | | 3-acetate | | |
| 255. | 58567-11-6 | Cyclododecane, | Boisambren Forte | 0.00433000 |
| | | (ethoxymethoxy)- | | |
| 256. | 94400-98-3 | Naphth[2,3- | Molaxone | 0.00425000 |
| | | b]oxirene, | | |
| | | 1a,2,3,4,5,6,7,7a- | | |
| | | octahydro- | | |
| | | 1a,3,3,4,6,6- | | |
| | | hexamethyl-, | | |
| | | (1aR,4S,7aS)-rel- | | |
| 257. | 79-69-6 | 3-Buten-2-one, 4- | alpha-Irone | 0.00419000 |
| | | (2,5,6,6-tetramethyl- | | |
| | | 2-cyclohexen-1-yl)- | | |
| 258. | 65442-31-1 | Quinoline, 6-(1- | Iso Butyl | 0.00408000 |
| | | methylpropyl)- | Quinoline | |
| 259. | 87731-18-8 | Carbonic acid, 4- | Violiff | 0.00401000 |
| | | cycloocten-1-yl | | |
| | | methyl ester | | |
| 260. | 173445-65-3 | 1H-Indene-5- | Hivernal (A- | 0.00392000 |
| | | propanal, 2,3- | isomer) | |
| | | dihydro-3,3- | | |
| | | dimethyl- | | |
| 261. | 23911-56-0 | Ethanone, 1-(3- | Nerolione | 0.00383000 |
| | | methyl-2- | | |
| | | benzofuranyl)- | | |
| 262. | 52474-60-9 | 3-Cyclohexene-1- | Precyclemone B | 0.00381000 |
| | | carboxaldehyde, 1- | | |
| | | methyl-3-(4-methyl- | | |
| | | 3-penten-1-yl)- | | |
| 263. | 139539-66-5 | 6-Oxabicyclo | Cassifix | 0.00381000 |
| | | [3.2.1]octane, 5- | | |
| | | methyl-1-(2,2,3- | | |
| | | trimethyl-3- | | |
| | | cyclopenten-1-yl)- | | |
| 264. | 80858-47-5 | Benzene, [2- | Phenafleur | 0.00380000 |
| | | (cyclohexyloxy)ethyl]- | | |
| 265. | 32764-98-0 | 2H-Pyran-2-one, | Jasmolactone | 0.00355000 |
| | | tetrahydro-6-(3- | | |
| | | penten-1-yl)- | | |
| 266. | 78417-28-4 | 2,4,7-Decatrienoic | Ethyl 2,4,7- | 0.00353000 |
| | | acid, ethyl ester | decatrienoate | |
| 267. | 140-26-1 | Butanoic acid, 3- | Beta Phenyl Ethyl | 0.00347000 |
| | | methyl-, 2- | Isovalerate | |
| | | phenylethyl ester | | |
| 268. | 105-90-8 | 2,6-Octadien-1-ol, | Geranyl | 0.003360000 |
| | | 3,7-dimethyl-, 1- | Propionate | |
| | | propanoate, (2E)- | | |
| 269. | 41816-03-9 | Spiro[1,4- | Rhubofix ® | 0.00332000 |
| | | methanonaphthalene- | | |
| | | 2(1H),2′-oxirane], | | |
| | | 3,4,4a,5,8,8a- | | |
| | | hexahydro-3′,7- | | |
| | | dimethyl- | | |
| 270. | 7070-15-7 | Ethanol, 2- | Arbanol | 0.00326000 |
| | | [[(1R,2R,4R)- | | |
| | | 1,7,7-trimethylbicy- | | |
| | | clo[2.2.1]hept- | | |
| | | 2-yl]oxy]-, | | |
| | | rel- | | |
| 271. | 93-29-8 | Phenol, 2-methoxy- | Iso Eugenol | 0.00324000 |
| | | 4-(1-propen-1-yl)-, | Acetate | |
| | | 1-acetate | | |
| 272. | 476332-65-7 | 2H-Indeno[4,5- | Amber Xtreme | 0.00323000 |
| | | b]furan, decahydro- | Compound 1 | |
| | | 2,2,6,6,7,8,8- | | |
| | | heptamethyl- | | |
| 273. | 68901-15-5 | Acetic acid, 2- | Cyclogalbanate | 0.00323000 |
| | | (cyclohexyloxy)-, 2- | | |
| | | propen-1-yl ester | | |
| 274. | 107-75-5 | Octanal, 7-hydroxy- | Hydroxycitronellal | 0.00318000 |
| | | 3,7-dimethyl- | | |
| 275. | 68611-23-4 | Naphtho[2,1- | Grisalva | 0.00305000 |
| | | b]furan, 9b- | | |
| | | ethyldodecahydro- | | |
| | | 3a,7,7-trimethyl- | | |
| 276. | 313973-37-4 | 1,6-Heptadien-3- | Pharaone | 0.00298000 |
| | | one, 2-cyclohexyl- | | |
| 277. | 137-00-8 | 5-Thiazoleethanol, | Sulfurol | 0.00297000 |
| | | 4-methyl- | | |
| 278. | 7779-30-8 | 1-Penten-3-one, 1- | Methyl Ionone | 0.00286000 |
| | | (2,6,6-trimethyl-2- | | |
| | | cyclohexen-1-yl)- | | |
| 279. | 127-51-5 | 3-Buten-2-one, 3- | Isoraldeine Pure | 0.00282000 |
| | | methyl-4-(2,6,6- | | |
| | | trimethyl-2- | | |
| | | cyclohexen-1-yl)- | | |
| 280. | 72903-27-6 | 1,4- | Fructalate ™ | 0.00274000 |
| | | Cyclohexanedicarboxylic | | |
| | | acid, 1,4- | | |
| | | diethyl ester | | |
| 281. | 7388-22-9 | 3-Buten-2-one, 4- | Ionone Gamma | 0.00272000 |
| | | (2,2-dimethyl-6- | Methyl | |
| | | methylenecyclohexyl)- | | |
| | | 3-methyl- | | |
| 282. | 104-67-6 | 2(3H)-Furanone, 5- | gamma- | 0.00271000 |
| | | heptyldihydro- | Undecalactone | |
| | | | (racemic) | |
| 283. | 1205-17-0 | 1,3-Benzodioxole-5- | Helional | 0.00270000 |
| | | propanal, α-methyl- | | |
| 284. | 33704-61-9 | 4H-Inden-4-one, | Cashmeran | 0.00269000 |
| | | 1,2,3,5,6,7- | | |
| | | hexahydro-1,1,2,3,3- | | |
| | | pentamethyl- | | |
| 285. | 36306-87-3 | Cyclohexanone, 4- | Kephalis | 0.00269000 |
| | | (1-ethoxyethenyl)- | | |
| | | 3,3,5,5-tetramethyl- | | |
| 286. | 97384-48-0 | Benzenepropanenitrile, | Citrowanil ® B | 0.00265000 |
| | | α-ethenyl-α- | | |
| | | methyl- | | |
| 287. | 141-13-9 | 9-Undecenal, 2,6,10- | Adoxal | 0.00257000 |
| | | trimethyl- | | |
| 288. | 2110-18-1 | Pyridine, 2-(3- | Corps Racine VS | 0.00257000 |
| | | phenylpropyl)- | | |
| 289. | 27606-09-3 | Indeno[1,2-d]-1,3- | Magnolan | 0.00251000 |
| | | dioxin, 4,4a,5,9b- | | |
| | | tetrahydro-2,4- | | |
| | | dimethyl- | | |
| 290. | 67634-20-2 | Propanoic acid, 2- | Cyclabute | 0.00244000 |
| | | methyl-, | | |
| | | 3a,4,5,6,7,7a- | | |
| | | hexahydro-4,7- | | |
| | | methano-1H-inden- | | |
| | | 5-yl ester | | |
| 291. | 65405-72-3 | 1-Naphthalenol, | Oxyoctaline | 0.00236000 |
| | | 1,2,3,4,4a,7,8,8a- | Formate | |
| | | octahydro-2,4a,5,8a- | | |
| | | tetramethyl-, 1- | | |
| | | formate | | |
| 292. | 122-40-7 | Heptanal, 2- | Amyl Cinnamic | 0.00233000 |
| | | (phenylmethylene)- | Aldehyde | |
| 293. | 103694-68-4 | Benzenepropanol, | Majantol ® | 0.00224000 |
| | | β,β,3-trimethyl- | | |
| 294. | 13215-88-8 | 2-Cyclohexene-1-one, | Tabanone Coeur | 0.00223000 |
| | | 4-(2-buten-1- | | |
| | | ylidene)-3,5,5- | | |
| | | trimethyl- | | |
| 295. | 25152-85-6 | 3-Hexen-1-ol, 1- | Cis-3-Hexenyl | 0.00203000 |
| | | benzoate, (3Z)- | Benzoate | |
| 296. | 406488-30-0 | 2-Ethyl-N-methyl-N- | Paradisamide | 0.00200000 |
| | | (m-tolyl)butanamide | | |
| 297. | 121-33-5 | Benzaldehyde, 4- | Vanillin | 0.00194000 |
| | | hydroxy-3-methoxy- | | |
| 298. | 77-54-3 | 1H-3a,7- | Cedac | 0.00192000 |
| | | Methanoazulen-6-ol, | | |
| | | octahydro-3,6,8,8- | | |
| | | tetramethyl-, 6- | | |
| | | acetate, | | |
| | | (3R,3aS,6R,7R,8aS)- | | |
| 299. | 76842-49-4 | 4,7-Methano-1H- | Frutene | 0.00184000 |
| | | inden-6-ol, | | |
| | | 3a,4,5,6,7,7a- | | |
| | | hexahydro-8,8- | | |
| | | dimethyl-, 6- | | |
| | | propanoate | | |
| 300. | 121-39-1 | 2-Oxiranecarboxylic | Ethyl Phenyl | 0.00184000 |
| | | acid, 3-phenyl-, | Glycidate | |
| | | ethyl ester | | |
| 301. | 211299-54-6 | 4H-4a,9- | Ambrocenide ® | 0.00182000 |
| | | Methanoazuleno[5,6- | | |
| | | d]-1,3-dioxole, | | |
| | | octahydro- | | |
| | | 2,2,5,8,8,9a- | | |
| | | hexamethyl-, | | |
| | | (4aR,5R,7aS,9R)- | | |
| 302. | 285977-85-7 | (2,5-Dimethyl-1,3- | Lilyflore | 0.00180000 |
| | | dihydroinden-2- | | |
| | | yl)methanol | | |
| 303. | 10094-34-5 | Butanoic acid, 1,1- | Dimethyl Benzyl | 0.00168000 |
| | | dimethyl-2- | Carbinyl Butyrate | |
| | | phenylethyl ester | | |
| 304. | 40785-62-4 | Cyclododeca[c] | Muscogene | 0.00163000 |
| | | furan, 1,3,3a,4,5,6,7, | | |
| | | 8,9,10,11,13a- | | |
| | | dodecahydro- | | |
| 305. | 75490-39-0 | Benzenebutanenitrile, | Khusinil | 0.00162000 |
| | | α,α,γ-trimethyl- | | |
| 306. | 55418-52-5 | 2-Butanone, 4-(1,3- | Dulcinyl | 0.00161000 |
| | | benzodioxol-5-yl)- | | |
| 307. | 3943-74-6 | Benzoic acid, 4- | Carnaline | 0.00157000 |
| | | hydroxy-3-methoxy-, | | |
| | | methyl ester | | |
| 308. | 72089-08-8 | 3-Cyclopentene-1- | Brahmanol ® | 0.00154000 |
| | | butanol, β,2,2,3- | | |
| | | tetramethyl-2- | | |
| | | Methyl-4-(2,2,3- | | |
| | | trimethyl-3- | | |
| | | cyclopenten-1- | | |
| | | yl)butanol | | |
| 309. | 3155-71-3 | 2-Butenal, 2-methyl- | Boronal | 0.00147000 |
| | | 4-(2,6,6-trimethyl-1- | | |
| | | cyclohexen-1-yl)- | | |
| 310. | 2050-08-0 | Benzoic acid, 2- | Amyl Salicylate | 0.00144000 |
| | | hydroxy-, pentyl | | |
| | | ester | | |
| 311. | 41199-20-6 | 2-Naphthalenol, | Ambrinol | 0.00140000 |
| | | decahydro-2,5,5- | | |
| | | trimethyl- | | |
| 312. | 12262-03-2 | ndecanoic acid, 3- | Iso Amyl | 0.00140000 |
| | | methylbutyl ester | Undecylenate | |
| 313. | 107-74-4 | 1,7-Octanediol, 3,7- | Hydroxyol | 0.00139000 |
| | | dimethyl- | | |
| 314. | 91-64-5 | 2H-1-Benzopyran-2- | Coumarin | 0.00130000 |
| | | one | | |
| 315. | 68901-32-6 | 1,3-Dioxolane, 2-[6- | Glycolierral | 0.00121000 |
| | | methyl-8-(1- | | |
| | | methylethyl)bicy- | | |
| | | clo[2.2,2]oct-5- | | |
| | | en-2-yl]- | | |
| 316. | 68039-44-1 | Propanoic acid, 2,2- | Pivacyclene | 0.00119000 |
| | | dimethyl-, 3a,4,5,6, | | |
| | | 7,7a-hexahydro-4,7- | | |
| | | methano-1H-inden- | | |
| | | 6-yl ester | | |
| 317. | 106-29-6 | Butanoic acid, (2E)- | Geranyl Butyrate | 0.00116000 |
| | | 3,7-dimethyl-2,6- | | |
| | | octadien-1-yl ester | | |
| 318. | 5471-51-2 | 2-Butanone, 4-(4- | Raspberry ketone | 0.00106000 |
| | | hydroxyphenyl)- | | |
| 319. | 109-42-2 | 10-Undecenoic acid, | Butyl | 0.00104000 |
| | | butyl ester | Undecylenate | |
| 320. | 2785-89-9 | 4-Ethyl-2- | 4-Ethylguaiacol | 0.02000000 |
| | | methoxyphenol | | |
| 321. | 27538-10-9 | 2-ethyl-4-hydroxy- | Homofuronol | 0.01210000 |
| | | 5-methylfuran-3-one | | |

*Vapor Pressures are acquired as described in the Test Methods Section.
**Origin: Same as for Table 2 hereinabove.

TABLE 3B
Moderate Volatile Natural Oils.

| No. | Natural oil | Supplier |
|---|---|---|
| 1. | Bay Oil Terpeneless | IFF |
| 2. | Cade Oil | H. Reynaud & Fils |
| 3. | Cedar Atlas Oil | Robertet |
| 4. | Cinnamon Bark Oil | Robertet |
| 5. | Cinnamon Oleoresin | Citrus & Allied Essences |
| 6. | Clove Bud Oil | Robertet |
| 7. | Clove Leaf Oil Rectified | H. Reynaud & Fils |
| 8. | Clove Stem Oil | H. Reynaud & Fils |
| 9. | Davana Oil | Robertet |
| 10. | Geranium Bourbon | Robertet |
| 11. | Ginger Oil Fresh Madagascar | IFF |
| 12. | Hay Absolute MD 50 PCT | IFF |
| 13. | Juniperberry Oil T'less | Robertet |
| 14. | Papyrus Oil | Robertet |
| 15. | Rose Absolute Oil | Robertet |
| 16. | Tonka Bean Absolute | Robertet |
| 17. | Wormwood Oil | Robertet |

Suppliers:
Citrus & Allied Essences, New York, USAH. Reynaud & Fils, Montbrun-les-Bains, France IFF, Hazlet, New Jersey, USA
Robertet, Grasse, France

Moderate volatile fragrance materials can be selected from the group of Tables 3A or 3B. However, it is understood by one skilled in the art that other moderate volatile fragrance materials, not recited in Tables 3A or 3B, would also fall within the scope of the present invention, so long as they have a vapor pressure of 0.1 to 0.001 Torr at 25° C.

High Volatile Fragrance Materials

The fragrance component includes at least one high volatile fragrance material having a vapor pressure greater than 0.1 Torr (0.0133 kPa) at 25° C. In some examples, the high volatile fragrance material can include at least 2 high volatile fragrance materials, 3 high volatile fragrance materials, or at least 5 high volatile fragrance materials, or at least 7 high volatile fragrance materials. In some embodiments, the high volatile fragrance component can be in a range of from about 0 wt % to about 100 wt % of the fragrance component, about 10 wt % to about 90 wt %, about 20 wt % to about 80 wt %, about 30 wt % to about 70 wt %, or about 40 wt % to about 60 wt %. The exact wt % of the high volatile fragrance component can vary greatly based on the fragrance's construction. For example, in some examples, where the high volatile fragrance material is present in a top-heavy construction, the high volatile fragrance material can be greater than about 30 wt % of the fragrance component, greater than about 40 wt %, greater than about 50 wt %, greater than about 60 wt %, about 31 wt % to about 60 wt %, about 40 wt % to about 50 wt %, or less than, equal to, or greater than about 30 wt %, 31, 35, 40, 45, 50, 55, 60, 65, 70, or 75 wt %. In some other examples, the high volatile fragrance material can be present in an amount ranging from about 0 wt % to about 30 wt % of the fragrance component, about 1 wt % to about 30 wt %, about 5 wt % to about 30 wt %, or less than, equal to, or greater than about 0 wt %, 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.25, 27, 27.5, 28, 28.5, 29, 29.5, or about 30 wt %. In still further embodiments, the high volatile fragrance material can be in an amount ranging from about 30 wt % to about 40 wt % of the fragrance component, about 32 wt % to about 35 wt %, less than, equal to, or greater than about 30 wt % 31, 32, 33, 34, 35, 36, 37, 38, 39, or 40 wt %. In some embodiments, the low-volatile fragrance material can be the only fragrance material present. If there are more than one high volatile fragrance materials, then the ranges provided hereinabove cover the total of all of the high volatile fragrance materials. Suitable examples of high volatile fragrances materials are provided in Tables 4A and 4B below.

In some embodiments, the high volatile fragrance materials can be present as a component of the coating composition as well. In these embodiments, the high volatile fragrance material can be present in a range of from about 0.01 wt % to about 50 wt %.

Preferably, the high volatile fragrance material is selected from at least 1 material, or at least 2 materials, or at least 3 materials, or at least 5 materials, at least 7 materials, or at least 9 high volatile fragrance materials as disclosed in Table 4A. Natural fragrance materials or oils having an aggregate vapor pressure greater than 0.1 Torr (0.0133 kPa) at 25° C. are provided in Table 4B. High Volatile Natural Oils. Relative to any other fragrance materials, the low volatile fragrance component can be present in a ratio ranging from about 20:1 to about 1:20, about 10:1 to about 1:10, about 5:1 to about 1:5, or about 1:1.

TABLE 4A
High Volatile Fragrance Materials

| | | | | Vapor Pressure |
|---|---|---|---|---|
| No. | CAS Number | IUPAC Name | Common Name** | (Torr at 25° C.)* |
| 1. | 107-31-3 | Formic acid, | Methyl Formate | 732.00000000 |
| | | methyl ester | | |
| 2. | 75-18-3 | Methane, 1,1′- | Dimethyl Sulfide | 647.00000000 |
| | | thiobis- | 1.0% In DEP | |
| 3. | 141-78-6 | Acetic acid ethyl | Ethyl Acetate | 112.00000000 |
| | | ester | | |
| 4. | 105-37-3 | Propanoic acid, | Ethyl Propionate | 44.50000000 |
| | | ethyl ester | | |
| 5. | 110-19-0 | Acetic acid, 2- | Isobutyl Acetate | 18.00000000 |
| | | methylpropyl | | |
| | | ester | | |
| 6. | 105-54-4 | Butanoic acid, | Ethyl Butyrate | 13.90000000 |
| | | ethyl ester | | |
| 7. | 14765-30-1 | 1-Butanol | Butyl Alcohol | 8.52000000 |
| 8. | 7452-79-1 | Butanoic acid, 2- | Ethyl-2-Methyl | 7.85000000 |
| | | methyl-, ethyl | Butyrate | |
| | | ester | | |
| 9. | 123-92-2 | 1-Butanol, 3- | Iso Amyl Acetate | 5.68000000 |
| | | methyl-, 1- | | |
| | | acetate | | |
| 10. | 66576-71-4 | Butanoic acid, 2- | Iso Propyl 2- | 5.10000000 |
| | | methyl-, 1- | Methylbutyrate | |
| | | methylethyl ester | | |
| 11. | 110-43-0 | 2-Heptanone | Methyl Amyl | 4.73000000 |
| | | | Ketone | |
| 12. | 6728-26-3 | 2-Hexenal, (2E)- | Trans-2 Hexenal | 4.62000000 |
| 13. | 123-51-3 | 1-Butanol, 3- | Isoamyl Alcohol | 4.16000000 |
| | | methyl- | | |
| 14. | 1191-16-8 | 2-Buten-1-ol, 3- | Prenyl acetate | 3.99000000 |
| | | methyl-, 1- | | |
| | | acetate | | |
| 15. | 57366-77-5 | 1,3-Dioxolane-2- | Methyl Dioxolan | 3.88000000 |
| | | methanamine, N- | | |
| | | methyl- | | |
| 16. | 7785-70-8 | Bicyclo[3.1.1]hept- | Alpha Pinene | 3.49000000 |
| | | 2-ene, 2,6,6- | | |
| | | trimethyl-, | | |
| | | (1R,5R)- | | |
| 17. | 79-92-5 | Bicyclo[2.2.1]heptane, | Camphene | 3.38000000 |
| | | 2,2-dimethyl-3- | | |
| | | methylene- | | |
| 18. | 94087-83-9 | 2-Butanethiol, 4- | 4-Methoxy-2- | 3.31000000 |
| | | methoxy-2- | Methyl-2- | |
| | | methyl- | Butanenthiol | |
| 19. | 39255-32-8 | Pentanoic acid, | Manzanate | 2.91000000 |
| | | 2-methyl-, ethyl | | |
| | | ester | | |
| 20. | 3387-41-5 | Bicyclo[3.1.0]hexane, | Sabinene | 2.63000000 |
| | | 4-methylene-1-(1- | | |
| | | methylethyl)- | | |
| 21. | 127-91-3 | Bicyclo[3.1.1]heptane, | Beta Pinene | 2.40000000 |
| | | 6,6-dimethyl-2- | | |
| | | methylene- | | |
| 22. | 105-68-0 | 1-Butanol, 3- | Amyl Propionate | 2.36000000 |
| | | methyl-, 1- | | |
| | | propanoate | | |
| 23. | 123-35-3 | 1,6-Octadiene, 7- | Myrcene | 2.29000000 |
| | | methyl-3- | | |
| | | methylene- | | |
| 24. | 124-13-0 | Octanal | Octyl Aldehyde | 2.07000000 |
| 25. | 7392-19-0 | 2H-Pyran, 2- | Limetol | 1.90000000 |
| | | ethenyltetrahydro- | | |
| | | 2,6,6-trimethyl- | | |
| 26. | 111-13-7 | 2-Octanone | Methyl Hexyl | 1.72000000 |
| | | | Ketone | |
| 27. | 123-66-0 | Hexanoic acid, | Ethyl Caproate | 1.66000000 |
| | | ethyl ester | | |
| 28. | 470-82-6 | 2- | Eucalyptol | 1.65000000 |
| | | Oxabicyclo[2.2.2]octane, | | |
| | | 1,3,3-trimethyl- | | |
| 29. | 99-87-6 | Benzene, 1- | Para Cymene | 1.65000000 |
| | | methyl-4-(1- | | |
| | | methylethyl)- | | |
| 30. | 104-93-8 | Benzene, 1- | Para Cresyl Methyl | 1.65000000 |
| | | methoxy-4- | Ether | |
| | | methyl- | | |
| 31. | 13877-91-3 | 1,3,6-Octatriene, | Ocimene | 1.56000000 |
| | | 3,7-dimethyl- | | |
| 32. | 138-86-3 | Cyclohexene, 1- | dl-Limonene | 1.54000000 |
| | | methyl-4-(1- | | |
| | | methylethenyl)- | | |
| 33. | 5989-27-5 | Cyclohexene, 1- | d-limonene | 1.54000000 |
| | | methyl-4-(1- | | |
| | | methylethenyl)-, | | |
| | | (4R)- | | |
| 34. | 106-68-3 | 3-Octanone | Ethyl Amyl Ketone | 1.50000000 |
| 35. | 110-41-8 | Undecanal, 2- | Methyl Nonyl | 1.43000000 |
| | | methyl- | Acetaldehyde | |
| 36. | 142-92-7 | Acetic acid, | Hexyl acetate | 1.39000000 |
| | | hexyl ester | | |
| 37. | 110-93-0 | 5-Hepten-2-one, | Methyl Heptenone | 1.28000000 |
| | | 6-methyl- | | |
| 38. | 81925-81-7 | 2-Hepten-4-one, | Filbertone 1% in | 1.25000000 |
| | | 5-methyl- | TEC | |
| 39. | 3681-71-8 | 3-Hexen-1-ol, 1- | cis-3-Hexenyl | 1.22000000 |
| | | acetate, (3Z)- | acetate | |
| 40. | 97-64-3 | Propanoic acid, | Ethyl Lactate | 1.16000000 |
| | | 2-hydroxy-, | | |
| | | ethyl ester | | |
| 41. | 586-62-9 | Cyclohexene, | Terpineolene | 1.13000000 |
| | | 1-methyl-4-(1- | | |
| | | methylethylidene)- | | |
| 42. | 51115-64-1 | Butanoic acid, 2- | Amyl butyrate | 1.09000000 |
| | | methylbutyl ester | | |
| 43. | 106-27-4 | Butanoic acid, 3- | Amyl Butyrate | 1.09000000 |
| | | methylbutyl ester | | |
| 44. | 99-85-4 | 1,4-Cyclohexadiene, | Gamma Terpinene | 1.08000000 |
| | | 1-methyl-4-(1- | | |
| | | methylethyl)- | | |
| 45. | 18640-74-9 | Thiazole, 2-(2- | 2-Isobutylthiazole | 1.07000000 |
| | | methylpropyl)- | | |
| 46. | 928-96-1 | 3-Hexen-1-ol, | cis-3-Hexenol | 1.04000000 |
| | | (3Z)- | | |
| 47. | 100-52-7 | Benzaldehyde | Benzaldehyde | 0.97400000 |
| 48. | 141-97-9 | Butanoic acid, 3- | Ethyl Acetoacetate | 0.89000000 |
| | | oxo-, ethyl ester | | |
| 49. | 928-95-0 | 2-Hexen-1-ol, | Trans-2-Hexenol | 0.87300000 |
| | | (2E)- | | |
| 50. | 928-94-9 | 2-Hexen-1-ol, | Beta Gamma | 0.87300000 |
| | | (2Z)- | Hexenol | |
| 51. | 24691-15-4 | Cyclohexane, 3- | Herbavert | 0.85200000 |
| | | ethoxy-1,1,5- | | |
| | | trimethyl-, cis- | | |
| | | (9CI) | | |
| 52. | 19872-52-7 | 2-Pentanone, 4- | 4-Methyl-4- | 0.84300000 |
| | | mercapto-4- | Mercaptopentan-2- | |
| | | methyl- | one 1 ppm TEC | |
| 53. | 3016-19-1 | 2,4,6-Octatriene, | Allo-Ocimene | 0.81600000 |
| | | 2,6-dimethyl-, | | |
| | | (4E,6E)- | | |
| 54. | 69103-20-4 | Oxirane, 2,2- | Myroxide | 0.80600000 |
| | | dimethyl-3-(3- | | |
| | | methyl-2,4- | | |
| | | pentadien-1-yl)- | | |
| 55. | 189440-77-5 | 4,7-Octadienoic | Anapear | 0.77700000 |
| | | acid, methyl | | |
| | | ester, (4E)- | | |
| 56. | 67633-96-9 | Carbonic acid, | Liffarome ™ | 0.72100000 |
| | | (3Z)-3-hexen-1- | | |
| | | yl methyl ester | | |
| 57. | 123-68-2 | Hexanoic acid, | Allyl Caproate | 0.67800000 |
| | | 2-propen-1-yl | | |
| | | ester | | |
| 58. | 106-72-9 | 5-Heptenal, 2,6- | Melonal | 0.62200000 |
| | | dimethyl- | | |
| 59. | 106-30-9 | Heptanoic acid, | Ethyl Oenanthate | 0.60200000 |
| | | ethyl ester | | |
| 60. | 68039-49-6 | 3-Cyclohexene-1- | Ligustral or Triplal | 0.57800000 |
| | | carboxaldehyde, | | |
| | | 2,4-dimethyl- | | |
| 61. | 101-48-4 | Benzene, (2,2- | Phenyl | 0.55600000 |
| | | dimethoxyethyl)- | Acetaldehyde | |
| | | | Dimethyl Acetal | |
| 62. | 16409-43-1 | 2H-Pyran, | Rose Oxide | 0.55100000 |
| | | tetrahydro-4- | | |
| | | methyl-2-(2- | | |
| | | methyl-1- | | |
| | | propen-1-yl)- | | |
| 63. | 925-78-0 | 3-Nonanone | Ethyl Hexyl Ketone | 0.55100000 |
| 64. | 100-47-0 | Benzonitrile | Benzyl Nitrile | 0.52400000 |
| 65. | 589-98-0 | 3-Octanol | Octanol-3 | 0.51200000 |
| 66. | 58430-94-7 | 1-Hexanol, | Iso Nonyl Acetate | 0.47000000 |
| | | 3,5,5-trimethyl-, | | |
| | | 1-acetate | | |
| 67. | 10250-45-0 | 4-Heptanol, | Alicate | 0.45400000 |
| | | 2,6-dimethyl-, | | |
| | | 4-acetate | | |
| 68. | 105-79-3 | Hexanoic acid, | Iso Butyl Caproate | 0.41300000 |
| | | 2-methylpropyl | | |
| | | ester | | |
| 69. | 2349-07-7 | Propanoic acid, | Hexyl isobutyrate | 0.41300000 |
| | | 2-methyl-, hexyl | | |
| | | ester | | |
| 70. | 23250-42-2 | Cyclohexanecar- | Cyprissate | 0.40500000 |
| | | boxylic acid, 1,4- | | |
| | | dimethyl-, | | |
| | | methyl ester, | | |
| | | trans- | | |
| 71. | 122-78-1 | Benzeneacetaldehyde | Phenyl acetaldehyde | 0.36800000 |
| 72. | 5405-41-4 | Butanoic acid, 3- | Ethyl-3-Hydroxy | 0.36200000 |
| | | hydroxy-, ethyl | Butyrate | |
| | | ester | | |
| 73. | 105-51-3 | Propanedioic | Diethyl Malonate | 0.34400000 |
| | | acid, 1,3-diethyl | | |
| | | ester | | |
| 74. | 93-58-3 | Benzoic acid, | Methyl Benzoate | 0.34000000 |
| | | methyl ester | | |
| 75. | 16356-11-9 | 1,3,5- | Undecatriene | 0.33600000 |
| | | Undecatriene | | |
| 76. | 65405-70-1 | 4-Decenal, (4E)- | Decenal (Trans-4) | 0.33100000 |
| 77. | 54546-26-8 | 1,3-Dioxane, 2- | Herboxane | 0.33000000 |
| | | butyl-4,4,6- | | |
| | | trimethyl- | | |
| 78. | 13254-34-7 | 2-Heptanol, 2,6- | Dimethyl-2 6- | 0.33000000 |
| | | dimethyl- | Heptan-2-ol | |
| 79. | 98-86-2 | Ethanone, 1- | Acetophenone | 0.29900000 |
| | | phenyl- | | |
| 80. | 93-53-8 | Benzeneacetaldehyde, | Hydratropic | 0.29400000 |
| | | α-methyl- | aldehyde | |
| 81. | 80118-06-5 | Propanoic acid, | Iso Pentyrate | 0.28500000 |
| | | 2-methyl-, 1,3- | | |
| | | dimethyl-3- | | |
| | | buten-1-yl ester | | |
| 82. | 557-48-2 | 2,6-Nonadienal, | E Z-2,6-Nonadien- | 0.28000000 |
| | | (2E,6Z)- | 1-al | |
| 83. | 24683-00-9 | Pyrazine, 2- | 2-Methoxy-3- | 0.27300000 |
| | | methoxy-3-(2- | Isobutyl Pyrazine | |
| | | methylpropyl)- | | |
| 84. | 104-57-4 | Formic acid, | Benzyl Formate | 0.27300000 |
| | | phenylmethyl | | |
| | | ester | | |
| 85. | 104-45-0 | Benzene, 1- | Dihydroanethole | 0.26600000 |
| | | methoxy-4- | | |
| | | propyl- | | |
| 86. | 491-07-6 | Cyclohexanone, | Iso Menthone | 0.25600000 |
| | | 5-methyl-2-(1- | | |
| | | methylethyl)-, | | |
| | | (2R,5R)-rel- | | |
| 87. | 89-80-5 | Cyclohexanone, | Menthone Racemic | 0.25600000 |
| | | 5-methyl-2-(1- | | |
| | | methylethyl)-, | | |
| | | (2R,5S)-rel- | | |
| 88. | 2463-53-8 | 2-Nonenal | 2 Nonen-1-al | 0.25600000 |
| 89. | 55739-89-4 | Cyclohexanone, | Thuyacetone | 0.25000000 |
| | | 2-ethyl-4,4- | | |
| | | dimethyl- | | |
| 90. | 150-78-7 | Benzene, 1,4- | Hydroquinone | 0.25000000 |
| | | dimethoxy- | Dimethyl Ether | |
| 91. | 64988-06-3 | Benzene, 1- | Rosacene | 0.24600000 |
| | | (ethoxymethyl)- | | |
| | | 2-methoxy- | | |
| 92. | 76-22-2 | Bicyclo[2,2.1]heptan- | Camphor gum | 0.22500000 |
| | | 2-one, | | |
| | | 1,7,7-trimethyl- | | |
| 93. | 67674-46-8 | 2-Hexene, 6,6- | Methyl | 0.21400000 |
| | | dimethoxy-2,5,5- | Pamplemousse | |
| | | trimethyl- | | |
| 94. | 112-31-2 | Decanal | Decyl Aldehyde | 0.20700000 |
| 95. | 16251-77-7 | Benzenepropanal, | Trifernal | 0.20600000 |
| | | β-methyl- | | |
| 96. | 93-92-5 | Benzenemethanol, | Methylphenyl- | 0.20300000 |
| | | α-methyl-, 1- | carbinol Acetate | |
| | | acetate | | |
| 97. | 143-13-5 | Acetic acid, | Nonyl Acetate | 0.19700000 |
| | | nonyl ester | | |
| 98. | 122-00-9 | Ethanone, 1-(4- | Para Methyl | 0.18700000 |
| | | methylphenyl)- | Acetophenone | |
| 99. | 24237-00-1 | 2H-Pyran, 6- | Gyrane | 0.18600000 |
| | | butyl-3,6- | | |
| | | dihydro-2,4- | | |
| | | dimethyl- | | |
| 100. | 41519-23-7 | Propanoic acid, | Hexenyl | 0.18200000 |
| | | 2-methyl-, (3Z)- | Isobutyrate | |
| | | 3-hexen-1-yl | | |
| | | ester | | |
| 101. | 93-89-0 | Benzoic acid, | Ethyl Benzoate | 0.18000000 |
| | | ethyl ester | | |
| 102. | 20780-48-7 | 3-Octanol, 3,7- | Tetrahydro Linalyl | 0.18000000 |
| | | dimethyl-, 3- | Acetate | |
| | | acetate | | |
| 103. | 101-41-7 | Methyl 2- | Methylphenyl | 0.17600000 |
| | | phenylacetate | acetate | |
| 104. | 40853-55-2 | 1-Hexanol, 5- | Tetrahydro | 0.17300000 |
| | | methyl-2-(1- | Lavandulyl | |
| | | methylethyl)-, 1- | Acetate | |
| | | acetate | | |
| 105. | 933-48-2 | Cyclohexanol, | Trimethylcyclo- | 0.17300000 |
| | | 3,3,5-trimethyl-, | hexanol | |
| | | (1R,5R)-rel- | | |
| 106. | 35158-25-9 | 2-Hexenal, 5- | Lactone of Cis | 0.17200000 |
| | | methyl-2-(1- | Jasmone | |
| | | methylethyl)- | | |
| 107. | 18479-58-8 | 7-Octen-2-ol, | Dihydromyrcenol | 0.16600000 |
| | | 2,6-dimethyl- | | |
| 108. | 140-11-4 | Acetic acid, | Benzyl acetate | 0.16400000 |
| | | phenylmethyl | | |
| | | ester | | |
| 109. | 14765-30-1 | Cyclohexanone, | 2-sec-Butyl Cyclo | 0.16300000 |
| | | 2-(1- | Hexanone | |
| | | methylpropyl)- | | |
| 110. | 20125-84-2 | 3-Octen-1-ol, | Octenol | 0.16000000 |
| | | (3Z)- | | |
| 111. | 142-19-8 | Heptanoic acid, | Allyl Heptoate | 0.16000000 |
| | | 2-propen-1-yl | | |
| | | ester | | |
| 112. | 100-51-6 | Benzenemethanol | Benzyl Alcohol | 0.15800000 |
| 113. | 10032-15-2 | Butanoic acid, 2- | Hexyl-2-Methyl | 0.15800000 |
| | | methyl-, hexyl | Butyrate | |
| | | ester | | |
| 114. | 695-06-7 | 2(3H)-Furanone, | Gamma | 0.15200000 |
| | | 5-ethyldihydro- | Hexalactone | |
| 115. | 21722-83-8 | Cyclohexaneethanol, | Cyclohexyl Ethyl | 0.15200000 |
| | | 1-acetate | Acetate | |
| 116. | 111-79-5 | 2-Nonenoic acid, | Methyl-2- | 0.14600000 |
| | | methyl ester | Nonenoate | |
| 117. | 16491-36-4 | Butanoic acid, | Cis 3 Hexenyl | 0.13500000 |
| | | (3Z)-3-hexen-1- | Butyrate | |
| | | yl ester | | |
| 118. | 111-12-6 | 2-Octynoic acid, | Methyl Heptine | 0.12500000 |
| | | methyl ester | Carbonate | |
| 119. | 59323-76-1 | 1,3-Oxathiane, | Oxane | 0.12300000 |
| | | 2-methyl-4- | | |
| | | propyl- (2R,4S)- | | |
| | | rel- | | |
| 120. | 62439-41-2 | Heptanal, 6- | Methoxy Melonal | 0.11900000 |
| | | methoxy-2,6- | | |
| | | dimethyl- | | |
| 121. | 13851-11-1 | Bicyclo[2,2.1]heptan- | Frenchyl Acetate | 0.11700000 |
| | | 2-ol, 1,3,3- | | |
| | | trimethyl-, 2- | | |
| | | acetate | | |
| 122. | 115-95-7 | 1,6-Octadien-3- | Linalyl acetate | 0.11600000 |
| | | ol, 3,7-dimethyl-, | | |
| | | 3-acetate | | |
| 123. | 18479-57-7 | 2-Octanol, 2,6- | Tetra-Hydro | 0.11500000 |
| | | dimethyl- | Myrcenol | |
| 124. | 78-69-3 | 3,7- | Tetra-Hydro | 0.11500000 |
| | | dimethyloctan-3- | Linalool | |
| | | ol | | |
| 125. | 111-87-5 | 1-Octanol | Octyl Alcohol | 0.11400000 |
| 126. | 71159-90-5 | 3-Cyclohexene- | Grapefruit | 0.10500000 |
| | | 1-methanethiol, | mercaptan | |
| | | α,α,4-trimethyl- | | |
| 127. | 80-25-1 | Cyclohexanemethanol, | Menthanyl Acetate | 0.10300000 |
| | | α,α,4-trimethyl-, 1- | | |
| | | acetate | | |
| 128. | 88-41-5 | Cyclohexanol, 2- | Verdox ™ | 0.10300000 |
| | | (1,1- | | |
| | | dimethylethyl)-, | | |
| | | 1-acetate | | |
| 129. | 32210-23-4 | Cyclohexanol, 4- | Vertenex | 0.10300000 |
| | | (1,1- | | |
| | | dimethylethyl)-, | | |
| | | 1-acetate | | |
| 130. | 112-44-7 | Undecanal | n-Undecanal | 0.10200000 |
| 131. | 124-19-6 | Nonanal | Nonanal Aldehyde | 0.53200000 |
| | | | C-9 | |
| 132. | 929253-05-4 | 6-methoxy-2,6- | 6-methoxy-2,6- | 0.04020000 |
| | | dimethyloctanal | dimethyl octanal | |
| 133. | 68039-47-4 | 2-propan-2- | Phenethyl Isopropyl | 0.24900000 |
| | | yloxyethylbenzene | Ether | |
| 134. | 6413-10-1 | ethyl 2(2- | Apple Ketal | 0.21900000 |
| | | methyl-1,3- | | |
| | | dioxolan-2- | | |
| | | yl)acetate | | |
| 135. | 106-23-0 | 3,7-dimethyloct- | citronellal | 0.21500000 |
| | | 6-enal | | |
| 136. | 14667-55-1 | Trimethyl | Trimethyl Pyrazine- | 1.72400000 |
| | | Pyrazine-2,3,5 | 2,3,5 | |

*Vapor Pressures are acquired as described in the Test Methods Section.
**Origin: Same as for Table 2 hereinabove.

TABLE 4B
High Volatile Fragrance Materials

| No. | Natural oil | Supplier |
|---|---|---|
| 1. | Angelica Seeds Oil | Robertet |
| 2. | Basil Oil Grand Vert | IFF |
| 3. | Bergamot Oil Reggio Early New Crop | Capua |
| 4. | Black Pepper Oil | Robertet |
| 5. | Blackcurrant Buds Absolute | Robertet |
| 6. | Cardamom Guatamala Extract CO2 | IFF |
| 7. | Cardamom Oil Guatemala | IFF |
| 8. | Cedarleaf Oil | Kerry |
| 9. | citronella oil | H. Reynaud & Fils |
| 10. | Clary Sage Oil French | IFF |
| 11. | Coffee Extract CO2 | Firmenich |
| 12. | Cucumber Extract | Firmenich |
| 13. | Cumin Oil | Robertet |
| 14. | Cypress Oil | IFF |
| 15. | Elemi Coeur Oil | Robertet |
| 16. | Ginger oil India | IFF |
| 17. | Grapefruit Zest | Citrus & Allied Essences |
| 18. | It. Bergamot Oil | Capua |
| 19. | Labdanum Cistus Absolute | Biolandes |
| 20. | Lavandin Grosso Oil | H. Reynaud & Fils |
| 21. | Lemon Oil Winter | Capua |
| 22. | Green Mandarin Oil | Simone Gatto |
| 23. | Nutmeg Oil | Robertet |
| 24. | Oil Orange Sinensal | Citrus & Allied Essences |
| 25. | Olibanum Oil Pyrogenous | Firmenich |
| 26. | Pepper Black CO2 Oil | Firmenich |
| 27. | Petitgrain Mandarinier Oil | Misitano & Stracuzzi |
| 28. | Pink Pepper CO2 OIL | Firmenich |
| 29. | Rum CO2 Oil | Firmenich |
| 30. | Sichuan Pepper CO2 oil | Firmenich |
| 31. | Styrax Resoid | IFF |
| 32. | Tangerine Oil | Robertet |
| 33. | Thym Oil | IFF |
| 34. | Violet Leaves Absolute | Robertet |

Suppliers
Biolandes, Le Sen, France
Capua, Campo Calabro, Italy
Citrus & Allied Essence, New York, USA
Firmenich, Geneva, Switzerland
Global Essence Inc, New Jersey, USA
H. Reynaud & Fils, Montbrun-les-Bains, France
IFF, Hazlet, New Jersey, USA
Kerry, Co. Kerry, Ireland
Mane, Le Bar-sur-Loup, France
Misitano & Stracuzzi, Messina, Italy
Robertet, Grasse, France
Simone Gatto, San Pierre Niceto, Italy

Exemplary high volatile fragrance materials selected from the group of Tables 4A or 4B are preferred. However, it is understood by one skilled in the art that other high volatile fragrance materials, not recited in Tables 4A or 4B, would also fall within the scope of the present invention, so long as they have a vapor pressure of greater than 0.1 Torr (0.0133 kPa) at 25° C.

Some embodiments can include a construction including a fragrance component present in an amount of from about 0.04 wt % to about 30 wt %, relative to the total weight of the composition. The fragrance component can include a glucam modulator and at least one low volatile fragrance material having a vapor pressure less than 0.001 Torr (0.000133 kPa) at 25° C. present in an amount of greater than about 30 wt % relative to the total weight of the fragrance component. At least one high volatile fragrance material having a vapor pressure greater than 0.1 Torr (0.0133 kPa) at 25° C. present in an amount greater than about 30 wt % relative to the total weight of the fragrance component. As well as at least one moderate volatile fragrance material having a vapor pressure in the range of 0.1 Torr to 0.001 Torr (0.0133 kPa to 0.000133 kPa) at 25° C. present in an amount of less than about 30 wt %, relative to the total weight of the fragrance component.

### Entrapment Materials

According to various embodiments, coating compositions, fragrance compositions, or both of the present invention can include an entrapment material at a level such that the weight ratio of the entrapment material to the coating or fragrance materials is in the range of from about 1:20 to about 20:1. In some examples, the composition may comprise an entrapment material present in the amount of from about 0.001 wt % to about 40 wt %, from about 0.1 wt % to about 25 wt %, from about 0.3 wt % to about 20 wt %, from about 0.5 wt % to about 10 wt %, or from about 0.75 wt % to about 5 wt %, relative to the total weight of the composition. The compositions disclosed herein may include from 0.001 wt % to 40%, from 0.1 wt % to 25 wt %, from 0.3 wt % to 20 wt %, from 0.5 wt % to 10 wt % or from 0.75 wt % to 5 wt %, relative to the total weight of the composition, of a cyclic oligosaccharide.

Suitable entrapment materials for use herein are selected from polymers; capsules, microcapsules and nanocapsules; liposomes, absorbents; cyclic oligosaccharides and mixtures thereof. Preferred are absorbents and cyclic oligosaccharides and mixtures thereof. Highly preferred are cyclic oligosaccharides (see PCT Publication Nos. WO2000/67721 (Procter & Gamble); and WO2000/67720 (Procter & Gamble); and U.S. Pat. No. 6,893,647 (Procter & Gamble)).

As used herein, the term “cyclic oligosaccharide” means a cyclic structure comprising six or more saccharide units. Preferred for use herein are cyclic oligosaccharides having six, seven or eight saccharide units and mixtures thereof, more preferably six or seven saccharide units and even more preferably seven saccharide units. It is common in the art to abbreviate six, seven and eight membered cyclic oligosaccharides to α, β and γ respectively.

The cyclic oligosaccharide of the compositions used for the present invention may comprise any suitable saccharide or mixtures of saccharides. Examples of suitable saccharides include, but are not limited to, glucose, fructose, mannose, galactose, maltose and mixtures thereof. However, preferred for use herein are cyclic oligosaccharides of glucose. The preferred cyclic oligosaccharides for use herein are α-cyclodextrins or β-cyclodextrins, or mixtures thereof, and the most preferred cyclic oligosaccharides for use herein are β-cyclodextrins.

The cyclic oligosaccharide, or mixture of cyclic oligosaccharides, for use herein may be substituted by any suitable substituent or mixture of substituents. Herein the use of the term “mixture of substituents” means that two or more different suitable substituents can be substituted onto one cyclic oligosaccharide. The derivatives of cyclodextrins consist mainly of molecules wherein some of the OH groups have been substituted. Suitable substituents include, but are not limited to, alkyl groups; hydroxyalkyl groups; dihydroxyalkyl groups; (hydroxyalkyl)alkylenyl bridging groups such as cyclodextrin glycerol ethers; aryl groups; maltosyl groups; allyl groups; benzyl groups; alkanoyl groups; cationic cyclodextrins such as those containing 2-hydroxy-3-(dimethylamino) propyl ether; quaternary ammonium groups; anionic cyclodextrins such as carboxyalkyl groups, sulphobutylether groups, sulphate groups, and succinylates; amphoteric cyclodextrins; and mixtures thereof.

The substituents may be saturated or unsaturated, straight chain, or branched chain. Preferred substituents include saturated and straight chain alkyl groups, hydroxyalkyl groups and mixtures thereof. Preferred alkyl and hydroxyalkyl substituents are selected from C₁-C₈ alkyl or hydroxyalkyl groups or mixtures thereof, more preferred alkyl and hydroxyalkyl substituents are selected from C₁-C₆ alkyl or hydroxyalkyl groups or mixtures thereof, even more preferred alkyl and hydroxyalkyl substituents are selected from C₁-C₄ alkyl or hydroxyalkyl groups and mixtures thereof. Especially preferred alkyl and hydroxyalkyl substituents are propyl, ethyl and methyl, more especially hydroxypropyl and methyl and even more preferably methyl.

Suitable cyclic oligosaccharides for use in the present invention are unsubstituted, or are substituted by only saturated straight chain alkyl, or hydroxyalkyl substituents. Therefore, preferred examples of cyclic oligosaccharides for use herein are α-cyclodextrin, β-cyclodextrin, methyl-α-cyclodextrin, methyl-β-cyclodextrin, hydroxypropyl-α-cyclodextrin and hydroxypropyl-β-cyclodextrin. Most preferred examples of cyclic oligosaccharides for use herein are methyl-α-cyclodextrin and methyl-β-cyclodextrin. These are available from Wacker-Chemie GmbH Hanns-Seidel-Platz 4, Munchen, DE under the tradename Alpha W6 M and Beta W7 M respectively.

The cyclic oligosaccharides of the compositions used for the present invention can be soluble in water, ethanol, or both water and ethanol. As used herein “soluble” means at least about 0.1 g of solute dissolves in 100 mL of solvent, at 25° C. and 1 standard atmospheric pressure (760 mmHg). The cyclic oligosaccharides for use herein have a solubility of at least about 1 g/100 mL, at 25° C. and 1 atm of pressure. In some examples, cyclic oligosaccharides are only present at levels up to their solubility limits in a given composition at room temperature. A person skilled in the art will recognize that the levels of cyclic oligosaccharides used in the present invention will also be dependent on the components of the composition and their levels, for example the solvents used or the exact fragrance oils, or combination of fragrance oils, present in the composition. Therefore, although the limits stated for the entrapment material are preferred, they are not exhaustive.

### Propellants

The coating compositions, fragrance compositions, or both described herein may include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42% by weight of the total fill of materials stored within the container.

### Antiperspirant Active

The fragrance compositions, coating compositions, or both described herein may be free of, substantially free of, or may include an antiperspirant active (e.g., any substance, mixture, or other material having antiperspirant activity). Examples of antiperspirant actives include astringent metallic salts, like the inorganic and organic salts of aluminum, zirconium and zinc, as well as mixtures thereof. Such antiperspirant actives include, for example, the aluminum and zirconium salts, such as aluminum halides, aluminum hydroxyhalides, zirconyl oxyhalides, zirconyl hydroxyhalides, and mixtures thereof.

### Other Ingredients

In yet another aspect, the fragrance compositions, coating compositions or both may consist essentially of the recited ingredients but may contain small amounts (not more than about 10 wt %, preferably no more than 5 wt %, or preferably no more than 2 wt % thereof, relative to the total weight of the composition) of other ingredients that do not impact on the fragrance profile, particularly the evaporation rate and release of the fragrance materials. For example, a fine fragrance composition may comprise stabilizing or anti-oxidant agents, UV filters or quenchers, or coloring agents, commonly used in perfumery. There are a number of other examples of additional ingredients that are suitable for inclusion in the present compositions, particularly in compositions for cosmetic use. These include, but are not limited to, alcohol denaturants such as denatonium benzoate; UV stabilizers such as benzophenone-2; antioxidants such as tocopheryl acetate; preservatives such as phenoxyethanol, benzyl alcohol, methyl paraben, and propyl paraben; dyes; pH adjusting agents such as lactic acid, citric acid, sodium citrate, succinic acid, phosphoric acid, sodium hydroxide, and sodium carbonate; deodorants and anti-microbials such as farnesol and zinc phenolsulphonate; humectants such as glycerine; oils; skin conditioning agents such as allantoin; cooling agents such as trimethyl isopropyl butanamide and menthol; silicones; solvents such as hexylene glycol; hair-hold polymers such as those described in PCT Publication No. WO94/08557 (Procter & Gamble); salts in general, such as potassium acetate and sodium chloride and mixtures thereof.

In yet another aspect, the composition of the present invention, depending on its intended use, is a mixture of fragrance materials possibly together with other ingredients such as, for example, perfume carriers. By the term “perfume carrier”, it is meant to include materials which are practically neutral from a perfumery point of view, e.g., which does not significantly alter the organoleptic properties of perfuming components. The perfume carrier may be a compatible liquid or solid fillers, diluents, and the like. The term “compatible”, as used herein, means that the components of the compositions of this invention are capable of being combined with the primary actives of the present invention, and with each other, in a manner such that there is no interaction which would substantially reduce the efficacy of the composition under ordinary use situations. The type of carrier utilized in the present invention depends on the type of product desired and may comprise, but are not limited to, solutions, aerosols, emulsions (including oil-in-water or water-in-oil), gels, and liposomes. Preferably, the carrier is a liquid and will be a solvent such as, for example, dipropyleneglycol, diethyl phthalate, isopropyl myristate, benzyl benzoate, 2-(2-ethoxyethoxy)-1-ethanol, or ethyl citrate (triethyl citrate).

In yet another aspect, the compositions for use in the present invention may take any form suitable for use, such as for perfumery or cosmetic use. These include, but are not limited to, vapor sprays, aerosols, emulsions, lotions, liquids, creams, gels, sticks, ointments, pastes, mousses, powders, granular products, substrates, cosmetics (e.g., semi-solid or liquid makeup, including foundations) and the like. In some examples, the compositions for use in the present invention take the form of a vapor spray. Compositions of the present invention can be further added as an ingredient to other compositions, preferably fine fragrance or cosmetic compositions, in which they are compatible. As such they can be used within a solid composition or applied substrates etc. Examples of products including the composition can include a fabric care product, an air care product, a home care product, a beauty care product, or a mixture thereof. Specific examples of products can include a perfume, an eau de toilette, an eau de parfum, a cologne, a body splash, a lotion, a cream, a shampoo, a conditioner, a hair mist, a body oil, a deodorant, a solid fragrance, or a body spray. The composition can be contacted with skin, hair, or a fabric.

### Article of Manufacture

The coating compositions, fragrance compositions, or both described herein may be included in an article of manufacture comprising a spray dispenser. The spray dispenser may comprise a vessel for containing the composition to be dispensed. The spray dispenser may comprise an aerosolized composition (e.g., a composition comprising a propellant) within the vessel as well. Other non-limiting examples of spray dispensers include non-aerosol dispensers (e.g., vapor sprays), manually activated dispensers, pump-spray dispensers, or any other suitable spray dispenser available in the art.

### Methods of Using the Compositions

The coating compositions of the present invention, according to any embodiment, can be useful to extend the amount of time, which the fragrances in the fragrance composition are detectable. This can be a result of the modulator in the coating composition diffusing at least partially into the fragrance composition. The coating composition can be configured as a primer, a top-coat, or both.

In embodiments in which the coating composition is configured as a primer, the coating composition can be applied directly to surfaces including human or animal skin or hair, paper (fragranced paper), air in a room (air freshener or aromatherapy composition), fabric, furnishings, dishes, hard surfaces and related materials. Preferred substrates include body surfaces such as, for example, hair and skin, most preferably skin. Following application, one or more of the fragrance compositions can be applied at least partially over the coating composition. Upon or shortly after contact, the modulator or modulators in the coating composition begin to diffuse into the fragrance composition.

Alternatively, in embodiments in which the coating composition is a topcoat, the coating composition can be applied to the fragrance compositions. The fragrance compositions of the present invention according to any embodiments described herein is a useful perfuming composition, which can be advantageously used as consumer products intended to perfume any suitable substrate such as the surfaces mentioned above.

The fragrance compositions of the present invention may be used in a conventional manner for fragrancing a substrate. An effective amount of the composition, such as from about 1 μL to about 100 mL, preferably from about 10 μL to about 1,000 μL, more preferably from about 25 μL to about 500 μL, from about 50 μL to about 100 μL, from about 100 μL to about 20 mL, or combinations thereof, is applied to the suitable substrate. Alternatively, an effective amount of the composition of the present invention is less than, equal to, or greater than about 1 μL, 10 μL, 25 μL or 50 μL to about 100 LL, 500 μL, 1,000 μL, 10,000 LL, 10 mL, 20 mL, 25 mL, 30 mL, 40 mL, 50 mL, 60 mL, 70 mL, 80 mL, 90 mL, or 100 mL. The composition may be applied by hand or applied utilizing a delivery apparatus such as, for example, vaporizer or atomizer. Preferably, the composition is allowed to dry after its application to the substrate. The scope of the present invention should be considered to cover one or more distinct applications of the composition or the continuous release of a composition via a vaporizer or other type of atomizer.

Following application of the fragrance compositions, the coating composition can be at least partially applied to the fragrance composition. Upon or shortly after contact, the modulator or modulators in the coating composition begin to diffuse into the fragrance composition.

The present disclosure provides a method for imparting, intensifying, or modifying an odor on human skin or human hair, comprising applying to human skin and/or human hair the composition of the present invention. Examples of notes or characters that can be enhanced include those chosen from a citrus-type note, green-type note, spicy-type note, cinnamon-type notes, pepper-type notes, cumin-type notes, ginger-type notes, floral-type notes, woody-type notes, cedarwood-type notes, sandalwood type notes, vetyver-type notes, leather-type note, smoky-type note, musk-type notes, and mixtures thereof.

Preferably, the fragrance profile or character of the composition of the present invention is detectable by a panel of experts or professional evaluators or individual experts or professional evaluators at later time points such as, for example, 30 mins, 1 hour, 2 hours, 3 hours, 4 hours, 5 hours, 6 hours, 7 hours, 8 hours, 10 hours, and possibly all the way up to 24 hours after application of the composition to a substrate as compared to controls (e.g., those without modulators). The nature of the fragrance which is applied prior or after the drying of the coating composition is not critical for the present disclosure. The technical teaching works for all fragrances independent if they are dedicated for males or females.

In another aspect, the present invention is also directed to a method of producing a consumer product comprising bringing into contact or mixing into the product an organoleptically active quantity of a composition of the present invention.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Test Method 1: Solubility Test

Fragrance ingredient was weighed, and added progressively to 5 gr of target solution. At each step, 0.050 gr of fragrance raw material was added, and shaken vigorously during 2 minutes to form a clear aqueous solution at room temperature. When insoluble, the mixture was allowed to sit for 8 hours to solubilize. The mixture was considered soluble when not appearing cloudy and no suspended oil or particles were visible after 8 hours.

TABLE 5
Evaluation of character difference (1 to 5 scale) of fragrances with coating compositions
versus fragrances without coating in fragrances with a hourglass-construction

| | | Coating | | Time | No | Mean | | |
|---|---|---|---|---|---|---|---|---|
| Fragrance | Type | Composition | Application | [h] | Evaluators | value | SD | SE |
| Lacoste | Male | IV | Primer | 0 | 3 | 2.33 | 0.58 | 0.33 |
| Essential | | | | | | | | |
| EDT | | | | | | | | |
| Lacoste | Male | IV | Primer | 3 | 3 | 3.00 | 1.00 | 0.58 |
| Essential | | | | | | | | |
| EDT | | | | | | | | |
| Lacoste | Male | IV | Primer | 0 | 3 | 2.67 | 0.58 | 0.33 |
| 1212 Noir | | | | | | | | |
| EDT | | | | | | | | |
| Lacoste | Male | IV | Primer | 3 | 3 | 3.00 | 0.00 | 0.00 |
| 1212 Noir | | | | | | | | |
| EDT | | | | | | | | |

Table 5 shows the effect of coating composition applied as a primer on two commercial products with a unique fragrance composition. These fragrances were constructed with a similar content of high- and low-volatility fragrance ingredients, and a lower content of mid-volatility fragrance ingredients as per the definitions in this patent. As it can be seen from the results, over time the difference in fragrance character becomes more evident. Surprisingly, the use of the primer composition enhances aromatic, citrus and woody notes, but reduced green notes in contrast to what is observed for other constructions.

### Example 2—Solubility Results

Classical perfume contain a high % of ethanol to solubilize perfume raw materials. Surprisingly, by the addition of a high amount of glucam, compensated by a removal of water, some raw materials like Ambroxan and Cedrol Crystals can be further solubilized as shown in Table 6. In the first case, solubility increased 6.49% whereas in the second one a 57.8%. Furthermore, natural raw materials like Coffee Santos SFE could be solubilized into glucam-containing composition. This gives the opportunity to create new olfactive spaces otherwise impossible.

TABLE 6
Comparison of fragrance material solubility between a classical
ethanol solution and a glucam-containing ethanol solution

| | | |
|---|---|---|
| | Max Solubility in | Max Solubility in |
| | 88% Ethanol/12% | 83% Ethanol/17% |
| Fragrance Material | Water solution | Glucam solution |

| | | | |
|---|---|---|---|
| Ambroxan | 0.847 | g | 0.902 g |
| Cerdol Crystals | 0.273 | g | 0.431 g |
| Coffee Santos SFE | 0 | g | 0.054 g |

### Additional Embodiments

The following exemplary embodiments are provided, the numbering of which is not to be construed as designating levels of importance:

Embodiment 1 provides a coating composition comprising:
a carrier;
a substantially non-odorous fragrance modulator present in the amount of from about 0.1 wt % to about 50 wt %, relative to the total weight of the composition; and
a fragrance material in a range of from about 0.01 wt % to about 50 wt %.

Embodiment 2 provides the coating composition of Embodiment 1, where the substantially non-odorous fragrance modulator is present in the amount of from about 5 wt % to about 20 wt %.

Embodiment 3 provides the coating composition of any one of Embodiments 1 or 2, wherein the at least one substantially non-odorous fragrance modulator is chosen from glucam, methyl glucoside polyol, ethyl glucoside polyol, propyl glucoside polyol, polypropylene glycol-10 methyl glucose ether, ethoxylated methyl glucose ether, polypropylene glycol-20 methyl glucose ether, caprylyl, capryl glucoside, undecyl glucoside, a cyclodextrine, an acrylate, a carboxylated acrylate/octyacrylamide copolymer, or mixtures thereof.

Embodiment 4 provides the coating composition of any one of Embodiments 1-3, wherein the at least one substantially non-odorous fragrance modulator is glucam.

Embodiment 5 provides the coating composition of any one of Embodiments 1-4, wherein the composition is substantially free of isocetyl alcohol, diisobutyl adipate, diisoamyl adipate, polypropylene glycol-3 myristyl ether, and neopentyl glycol diethyl hexanoate, neopentyl glycol diisononanoate, cetearyl ethyl hexanoate, and their mixtures, or a mixture thereof.

Embodiment 6 provides the coating composition of any one of Embodiments 1-5, wherein the at least one substantially non-odorous fragrance modulator is chosen from:
a compound of formula (I):

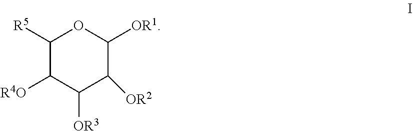

wherein:
   R¹ is hydrogen, alkyl, alkenyl or alkynyl;
   R² is selected from hydrogen, (C₂-C₂₀)alkyl, (C₂-C₂₀)alkenyl, (C₂-C₂₀)alkynyl, —[R⁶R⁷(R⁸)0]_{w}R⁹, wherein w is from 1 to 10, preferably 2 to 9;
   R³ is selected from hydrogen, alkyl, alkenyl, alkynyl, —[R⁶R\R⁸)0]yR⁹, wherein y is
from 1 to 10 or 2 to 9;
   R⁴ is selected from hydrogen, alkyl, alkenyl, alkynyl, —[R⁶R\R⁸)0]xR⁹, wherein x is from 1 to 10, preferably 2 to 9;
   R⁵ is selected from hydrogen, alkyl, alkenyl, alkynyl, —R⁶0 R⁹, —R⁶0 [R⁶R⁷(R⁸)0]zR⁹,
   wherein z is from 1 to 10, preferably 2 to 9;
   each R⁶ and R⁷ are independently selected from (C₂-C₂₀)alkylene, (C₂-C₂₀)alkenylene, or (C₂-C₂₀)alkynylene; and
   each R⁸ and R⁹ is independently selected from hydrogen or alkyl, a compound of formula (II):

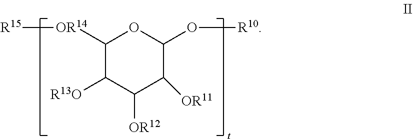

wherein:
   R¹⁰ is hydrogen, (C₂-C₂₀)alkyl, (C₂-C₂₀)alkenyl or (C₂-C₂₀)alkynyl;
   each R¹¹ is independently selected from hydrogen, (C₂-C₂₀)alkyl, (C₂-C₂₀)alkenyl, (C₂-C₂₀)alkynyl;
   each R¹² is independently selected from hydrogen, (C₂-C₂₀)alkyl, (C₂-C₂₀)alkenyl, or (C₂-C₂₀)alkynyl;
   each R¹³ is independently selected from hydrogen, (C₂-C₂₀)alkyl, (C₂-C₂₀)alkenyl, or (C₂-C₂₀)alkynyl;
   each R¹⁴ is selected from (C₂-C₂₀)alkylene, (C₂-C₂₀)alkenylene, or (C₂-C₂₀)alkynylene; and
   R¹⁵ is hydrogen, (C₂-C₂₀)alkyl, (C₂-C₂₀)alkenyl or (C₂-C₂₀) alkynyl;
wherein tis 5 or less, preferably 1, 2 or 3;
Sucrose Laurate, Sucrose Dilaurate, Sucrose Myristate, Sucrose Palmitate, Sucrose Stearate, Sucrose Distearate, Sucrose Tristearate, and their mixtures;
Trimethylcyclohexane derivatives having the formula (III):

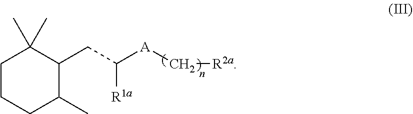

wherein:
   n is 0, 1 or 2;
   A is C═O or CH—OH;
   R^{1a} is hydrogen or methyl;
   R^{2a} is a C₂-C₁₀ hydrocarbon group; and
      is a saturated or unsaturated carbon-carbon bond;
L-menthoxy ether derivatives having the formula (IV):

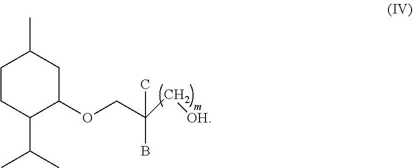

wherein:
   m is 0, 1 or 2;
   B is hydrogen or OH; and
   C is hydrogen or methyl;
Tetra-hydronaphthalene derivatives having the formula (V):

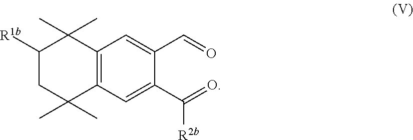

wherein:
   R^{1b} is hydrogen or methyl; and
   R^{2b} is alkyl;
140
Hyaluronic acid disaccharide sodium salt, sodium hyaluronate and their mixtures;
Ether derivatives having the formula (VI) or formula (VII):

C₅HₗOₘ—(OR^{1c})ₙ (VI).
wherein:
   C₅HₗOₘ is a pentose residue, wherein l is an integer from 6 to 9, and m is an integer from 1 to 4;
   n is an integer from 1 to 4; and
   R^{1c} is C₄-C₂₀ hydrocarbon group; and

C₅H_{c}O_{d}—(OCH₂CH₂—O—CH₂CH₂—O—R^{1c})ₑ (VII).
wherein:
   C₆HₓO_{y} is a hexose residue, wherein x is an integer from 7 to 11, and y is an integer from 1 to 5;
   z is an integer from 1 to 5; and
   R^{1d} is C₄-C₂₀ hydrocarbon group; and
Diethylene Glycol Ether derivatives having the formula (VITI) or formula (IX):

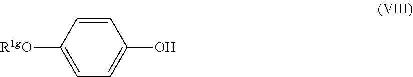

wherein:
   C₅H_{c}O_{d} is a pentose residue, wherein c is an integer from 6 to 8, and d
   is an integer from 1 to 3;
   e is an integer from 2 to 4; and
   R^{1e} is C₁-C₆ alkyl group; and

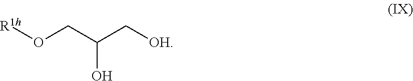

wherein:
   C₆H_{f}O_{g} is a hexose residue, wherein f is an integer from 7 to 10, and g is an integer from 1 to 4;
   h is an integer from 2 to 5; and
   R^{1f} is C₁-C₆ alkyl group;
   Hydroquinone Glycoside derivatives having the formula (X):

R¹ⁱOCOR²ⁱCOOR³ⁱ (X).
wherein:
   R^{1g} is selected from the group consisting of: (i) pentose residue, hexose residue, aminosaccharide residue, uronic acid residue and their mixtures; (ii) methylated versions of group (i); and (iii) mixtures of groups (i) and (ii); and Propylene Glycol Propyl Ether; Dicetyl Ether; Polyglycerin-4 Ethers; Isoceteth-5; Isoceteth-7, Isoceteth-10; Isoceteth-12; Isoceteth-15; Isoceteth-20; Isoceteth-25; Isoceteth-30; Disodium Lauroamphodipropionate; Hexaethylene glycol monododecyl ether; and their mixtures;
Glyceryl Ether derivatives having the formula (XI):

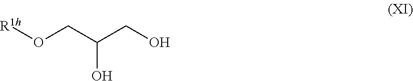

wherein:
   R^{1h} is C₄-C₁₂ aliphatic hydrocarbon group;
Panthenol Ethyl Ether, DL-Panthenol and their mixtures;
Aliphatic Dibasic Acid Diester derivatives having the formula (XII):

R¹ⁱOCOR²ⁱCOOR³ⁱ (XII).
wherein:
   R¹ⁱ is C₄-C₅ alkyl;
   R²ⁱ is C₄ alkylene; and
   R³ⁱ is C₄-C₅ alkyl; and
Aliphatic Ether derivatives having the formula (XIII):

R⁴ⁱ—O—(CH(CH₃)—CH₂O)ₐ—(CH₂—CH₂O)_{b}—H (XIII).
wherein:
   a and b are integers such that the sum of a and b is from 1 to 4; and
   R⁴ⁱ is an aliphatic chain comprising from 8 to 18 carbons;
N-hexadecyl n-nonanoate, Noctadecyl n-nonanoate and their mixtures;
Tricyclodecane Amide derivatives selected from the group consisting of:
   the compounds of formula (XIV):

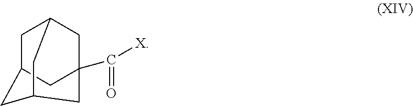

wherein:
   X is selected from:

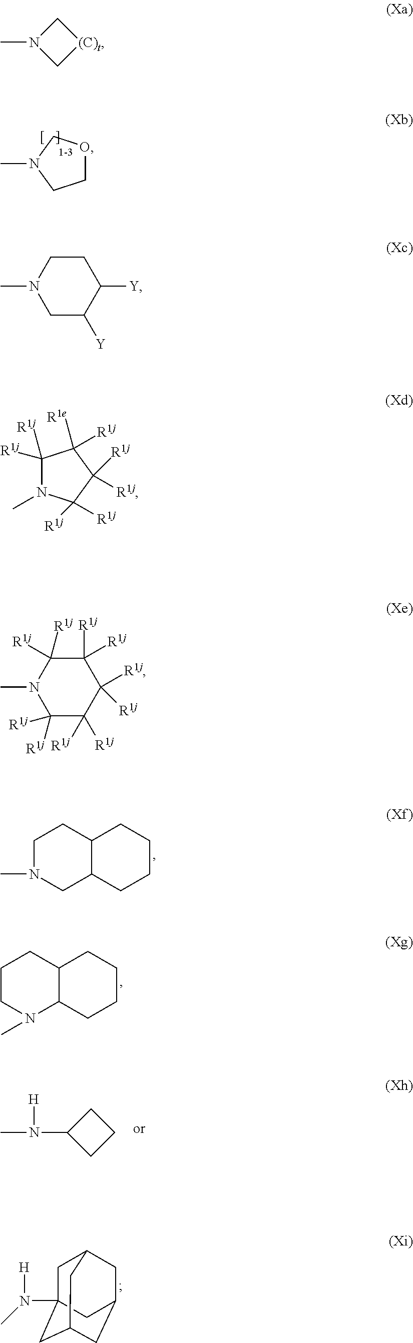

t is 1 to 8;
   Y is hydrogen, or a halogen; and
   each R^{1j} is independently selected from a hydrogen, or C₁-C₄ alkyl; the compounds of formula (XV):

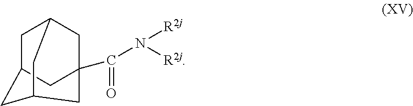

wherein:
   each R^{2j} is independently selected from a hydrogen, methyl, ethyl or C₃-C₁₈ alkyl, cycloalkyl or cycloheteroalkyl, with the proviso that both R^{2e} groups are not hydrogen; and
mixtures of the compounds of formulae (XII) and (XIII); and
mixtures thereof.

Embodiment 7 provides the coating composition of any one of Embodiments 1-6, wherein the coating composition comprises a plurality of modulators.

Embodiment 8 provides the coating composition of any one of Embodiments 1-7, wherein the carrier is an aqueous carrier or an organic carrier.

Embodiment 9 provides the coating composition of Embodiment 8, wherein the aqueous carrier is water.

Embodiment 10 provides the coating composition of any one of Embodiments 1-9, wherein the organic carrier is ethanol, dipropylene glycol, benzyl benzoate, diethyl phthalate, isopropyl myristate, (C₁-C₂₀)alkyl, (C₁-C₂₀)alkenyl, (C₁-C₂₀)alkynyl, (C₁-C₂₀)hydroxyl, or mixtures thereof.

Embodiment 11 provides the coating composition of any one of Embodiments 1-10, further comprising; a chelator, a thickener, an antioxidant, an emulsifier, or a combination thereof.

Embodiment 12 provides the coating composition of any one of Embodiments 1-11, further comprising at least one capsule dispersed in the composition.

Embodiment 13 provides the coating composition of Embodiment 12, wherein the at least one capsule comprises a fragrance component located therein.

Embodiment 14 provides the coating composition of any one of Embodiments 12 or 13, wherein the at least one capsule comprises a capsule wall comprising polyacrylates; polyethylenes; polyamides; polystyrenes; polyisoprenes; polycarbonates; polyesters; polyureas; polyurethanes; polyolefins; polysaccharides; epoxy resins; vinyl polymers; urea cross-linked with formaldehyde or gluteraldehyde; melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization; silk; wool; gelatine; cellulose; proteins; and combinations thereof.

Embodiment 15 provides the coating composition of any one of Embodiments 12-14, wherein the at least one capsule is configured to degrade.

Embodiment 16 provides the coating composition of any one of Embodiments 1-15, wherein the fragrance material is present in a range of from about 0.01 wt % to about 50 wt %

Embodiment 17 provides the coating composition of any one of Embodiments 1-16, wherein the coating composition is a primer or a top-coat.

Embodiment 18 provides the coating composition of any one of Embodiments 1-17, wherein the fragrance material comprises:
at least one low volatile fragrance material having a vapor pressure less than 0.001 Torr (0.000133 kPa) at 25° C.;
at least one moderate volatile fragrance material having a vapor pressure in the range of 0.1 Torr to 0.001 Torr (0.0133 kPa to 0.000133 kPa) at 25° C.;
at least one high volatile fragrance material having a vapor pressure greater than 0.1 Torr (0.0133 kPa) at 25° C.; or mixtures thereof.

Embodiment 19 provides the coating composition of Embodiment 18, wherein the high volatile fragrance material is chosen from any of the materials or combinations of materials listed in any one of Tables 4A and 4B.

Embodiment 20 provides the coating composition of any one of Embodiments 18 or 19, wherein the low volatile fragrance material is chosen from chosen from any of the materials or combinations of materials listed in any one of Tables 2A and 2B.

Embodiment 21 provides the coating composition of any one of Embodiments 18-20, wherein the moderate volatile fragrance material is chosen from chosen from any of the materials or combinations of materials listed in any one of Tables 3A and 3B.

Embodiment 22 provides a multi-component fragrance composition comprising:
the coating composition according to any one of Embodiments 1-21;
a fragrance composition in contact with the coating composition.

Embodiment 23 provides the multi-component fragrance composition of Embodiment 22, wherein the fragrance composition comprises:
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C. present in an amount greater than 30 wt %, relative to the total weight of the fragrance component;
   at least one moderate volatile fragrance material having a vapor pressure in the range of 0.1 Torr to 0.001 Torr (0.0133 kPa to 0.000133 kPa) at 25° C. present in an amount of from about 30 wt % to about 70 wt %, relative to the total weight of the fragrance component; and
   at least one high volatile fragrance material having a vapor pressure greater than 0.1 Torr (0.0133 kPa) at 25° C. present in an amount of from about 0 wt % to about 30 wt % relative to the total weight of the fragrance component.

Embodiment 24 provides the multi-component fragrance composition of Embodiment 23, wherein the fragrance composition further comprises at least one substantially non-odorous fragrance modulator present in the amount of from about 0.1 wt % to about 20 wt %, relative to the total weight of the composition.

Embodiment 25 provides the multi-component fragrance composition of any one of Embodiments 23 or 24, wherein the at least one low volatile fragrance material is present in an amount of from 31 wt % to about 60 wt % relative to the total weight of the fragrance material.

Embodiment 26 provides the multi-component fragrance composition of any one of Embodiments 23-25, wherein the at least one moderate volatile fragrance material is present in an amount of from about 35 wt % to about 60 wt % relative to the total weight of the fragrance material.

Embodiment 27 provides the multi-component fragrance composition of any one of Embodiments 23-26, wherein the at least one high volatile fragrance material is present in an amount of from about 1 wt % to about 30 wt % relative to the total weight of the fragrance material.

Embodiment 28 provides the multi-component fragrance composition of any one of Embodiments 23-27, wherein the high volatile fragrance material is chosen from any of the materials or combinations of materials listed in any one of Tables 4A and 4B.

Embodiment 29 provides the multi-component fragrance composition of any one of Embodiments 23-28, wherein the low volatile fragrance material is chosen from chosen from any of the materials or combinations of materials listed in any one of Tables 2A and 2B.

Embodiment 30 provides the multi-component fragrance composition of any one of Embodiments 23-29, wherein the moderate volatile fragrance material is chosen from chosen from any of the materials or combinations of materials listed in any one of Tables 3A and 3B.

Embodiment 31 provides the multi-component fragrance composition of Embodiment 17, wherein the fragrance composition comprises;
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C. present in an amount of from about 0 wt % to about 30 wt % relative to the total weight of the fragrance component;
   at least one moderate volatile fragrance material having a vapor pressure in the range of 0.1 Torr to 0.001 Torr (0.0133 kPa to 0.000133 kPa) at 25° C. present in an amount of from about 30 wt % to about 70 wt %, relative to the total weight of the fragrance component;
   at least one high volatile fragrance material having a vapor pressure greater than 0.1 Torr (0.0133 kPa) at 25° C. present in an amount greater than about 30 wt % relative to the total weight of the fragrance component or;
   a mixture thereof.

Embodiment 32 provides the multi-component fragrance composition of Embodiment 31, wherein the fragrance composition further comprises at least one substantially non-odorous fragrance modulator present in the amount of from about 0.1 wt % to about 20 wt %, relative to the total weight of the composition.

Embodiment 33 provides the multi-component fragrance composition of any one of Embodiments 31 or 32, wherein the at least one high volatile fragrance material is present in an amount of from 31 wt % to about 60 wt % relative to the total weight of the fragrance material.

Embodiment 34 provides the multi-component fragrance composition of any one of Embodiments 31-33, wherein the at least one moderate volatile fragrance material is present in an amount of from about 35 wt % to about 60 wt % relative to the total weight of the fragrance material.

Embodiment 35 provides the multi-component fragrance composition of any one of Embodiments 31-34, wherein the at least one low volatile fragrance material is present in an amount of from about 1 wt % to about 30 wt % relative to the total weight of the fragrance material.

Embodiment 36 provides the multi-component fragrance composition of any one of Embodiments 3135, wherein the high volatile fragrance material is chosen from any of the materials or combinations of materials listed in any one of Tables 4A and 4B.

Embodiment 37 provides the multi-component fragrance composition of any one of Embodiments 31-36, wherein the low volatile fragrance material is chosen from chosen from any of the materials or combinations of materials listed in any one of Tables 2A and 2B.

Embodiment 38 provides the multi-component fragrance composition of any one of Embodiments 18-37, wherein the moderate volatile fragrance material is chosen from chosen from any of the materials or combinations of materials listed in any one of Tables 3A and 3B.

Embodiment 39 provides the multi-component fragrance composition of Embodiment 17, wherein the fragrance composition comprises:
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C. present in an amount or from about 30 wt % to about 70 wt %, relative to the total weight of the fragrance component;
   at least one moderate volatile fragrance material having a vapor pressure in the range of 0.1 Torr to 0.001 Torr (0.0133 kPa to 0.000133 kPa) at 25° C. present in an amount less than about 30 wt %, relative to the total weight of the fragrance component; and
   at least one high volatile fragrance material having a vapor pressure greater than 0.1 Torr (0.0133 kPa) at 25° C. present in an amount of from about 0.1 wt % to about 30 wt % relative to the total weight of the fragrance component.

Embodiment 40 provides the multi-component fragrance composition of Embodiment 39, wherein the fragrance composition further comprises at least one substantially non-odorous fragrance modulator present in the amount of from about 0.1 wt % to about 20 wt %, relative to the total weight of the composition.

Embodiment 41 provides the multi-component fragrance composition of any one of Embodiments 39 or 40, wherein the at least one moderate volatile fragrance material is present in an amount of from 1 wt % to about 30 wt % relative to the total weight of the fragrance material.

Embodiment 42 provides the multi-component fragrance composition of any one of Embodiments 39-40, wherein the at least one high volatile fragrance material is present in an amount of from about 35 wt % to about 60 wt % relative to the total weight of the fragrance material.

Embodiment 43 provides the multi-component fragrance composition of any one of Embodiments 39-42, wherein the at least one low volatile fragrance material is present in an amount of from about 1 wt % to about 30 wt % relative to the total weight of the fragrance material.

Embodiment 44 provides the multi-component fragrance composition of any one of Embodiments 39-43, wherein the high volatile fragrance material is chosen from any of the materials or combinations of materials listed in any one of Tables 4A and 4B.

Embodiment 45 provides the multi-component fragrance composition of any one of Embodiments 39-44, wherein the low volatile fragrance material is chosen from chosen from any of the materials or combinations of materials listed in any one of Tables 2A and 2B.

Embodiment 46 provides the multi-component fragrance composition of any one of Embodiments 39-45, wherein the moderate volatile fragrance material is chosen from chosen from any of the materials or combinations of materials listed in any one of Tables 3A and 3B.

Embodiment 47 provides the multi-component fragrance composition of Embodiment 17, wherein the fragrance composition comprises;
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C.;
   at least one moderate volatile fragrance material having a vapor pressure in the range of 0.1 Torr to 0.001 Torr (0.0133 kPa to 0.000133 kPa) at 25° C.; and
   at least one high volatile fragrance material having a vapor pressure greater than 0.1 Torr (0.0133 kPa) at 25° C.,
   wherein the at least one low volatile fragrance material, at least one moderate volatile fragrance material, and at least one high volatile fragrance material are individually present in substantially the same amount relative to the total weight of the fragrance component.

Embodiment 48 provides the multi-component fragrance composition of Embodiment 47, wherein the composition maintains a perceived fragrance profile, particularly the characters attributable to the moderate volatile fragrance material, low volatile fragrance material, or high volatile fragrance material, to remain substantively unchanged from application up to 2 hrs, 3 hrs, 4 hrs, 5 hrs, 6 hrs or 8 hrs after application vs. a control composition that does not include the coating.

Embodiment 49 provides the multi-component fragrance composition of any one of Embodiments 47 or 48, wherein the composition maintains a perceived fragrance profile, particularly the characters attributable to the moderate volatile fragrance material, low volatile fragrance material, or high volatile fragrance material, to be substantially enhanced from application up to 2 hrs, 3 hrs, 4 hrs, 5 hrs, 6 hrs or 8 hrs after application vs. a control composition that does not include the coating.

Embodiment 50 provides the multi-component fragrance composition of Embodiment 17, wherein the fragrance composition comprises.
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C. present in an amount of greater than about 30 wt % relative to the total weight of the fragrance component;
   at least one high volatile fragrance material having a vapor pressure greater than 0.1 Torr (0.0133 kPa) at 25° C. present in an amount greater than about 30 wt % relative to the total weight of the fragrance component; and
   at least one moderate volatile fragrance material having a vapor pressure in the range of 0.1 Torr to 0.001 Torr (0.0133 kPa to 0.000133 kPa) at 25° C. present in an amount of less than the low volatile fragrance component or high fragrance component, relative to the total weight of the fragrance component.

Embodiment 51 provides the multi-component fragrance composition of Embodiment 50, wherein the fragrance composition further comprises at least one substantially non-odorous fragrance modulator present in the amount of from about 0.1 wt % to about 20 wt %, relative to the total weight of the composition.

Embodiment 52 provides the multi-component fragrance composition of any one of Embodiments 50 or 51, wherein the at least one high volatile fragrance material is present in an amount of from 31 wt % to about 60 wt % relative to the total weight of the fragrance material.

Embodiment 53 provides the multi-component fragrance composition of any one of Embodiments 50-52, wherein the at least one moderate volatile fragrance material is present in an amount of from about 1 wt % to about 29 wt % relative to the total weight of the fragrance material.

Embodiment 54 provides the multi-component fragrance composition of any one of Embodiments 50-53, wherein the at least one low volatile fragrance material is present in an amount of from about 30 wt % to about 60 wt % relative to the total weight of the fragrance material.

Embodiment 55 provides the multi-component fragrance composition of any one of Embodiments 50-54, wherein the high volatile fragrance material is chosen from any of the materials or combinations of materials listed in any one of Tables 4A and 4B.

Embodiment 56 provides the multi-component fragrance composition of any one of Embodiments 50-55, wherein the low volatile fragrance material is chosen from chosen from any of the materials or combinations of materials listed in any one of Tables 2A and 2B.

Embodiment 57 provides the multi-component fragrance composition of any one of Embodiments 50-56, wherein the moderate volatile fragrance material is chosen from chosen from any of the materials or combinations of materials listed in any one of Tables 3A and 3B.

Embodiment 58 provides the multi-component fragrance composition of Embodiment 17, wherein the fragrance composition comprises:
a fragrance component present in an amount of from about 0.04 wt % to about 30 wt %, relative to the total weight of the composition, and wherein the fragrance component comprises at least one of:
   at least one low volatile fragrance material having a vapor pressure less than 0.001 Torr (0.000133 kPa) at 25° C. present in an amount of from about 30 wt % to about 40 wt %, relative to the total weight of the fragrance component;
   at least one moderate volatile fragrance material having a vapor pressure in the range of 0.1 Torr to 0.001 Torr (0.0133 kPa to 0.000133 kPa) at 25° C. present in an amount of from about 30 wt % to about 40 wt %, relative to the total weight of the fragrance component; and
   at least one high volatile fragrance material having a vapor pressure greater than 0.1 Torr (0.0133 kPa) at 25° C. present in an amount of from about 30 wt % to about 40 wt %, relative to the total weight of the fragrance component.

Embodiment 59 provides the multi-component fragrance composition of Embodiment 58, wherein the fragrance composition further comprises at least one substantially non-odorous fragrance modulator present in the amount of from about 0.1 wt % to about 20 wt %, relative to the total weight of the composition.

Embodiment 60 provides the multi-component fragrance composition of any one of Embodiments 58 or 59, wherein the at least one high volatile fragrance material is present in an amount of from 32 wt % to about 35 wt % relative to the total weight of the fragrance material.

Embodiment 61 provides the multi-component fragrance composition of any one of Embodiments 58-60, wherein the at least one moderate volatile fragrance material is present in an amount of from about 32 wt % to about 35 wt % relative to the total weight of the fragrance material.

Embodiment 62 provides the multi-component fragrance composition of any one of Embodiments 58-61, wherein the at least one low volatile fragrance material is present in an amount of from about 32 wt % to about 35 wt % relative to the total weight of the fragrance material.

Embodiment 63 provides the multi-component fragrance composition of any one of Embodiments 58-62, wherein the high volatile fragrance material is chosen from any of the materials or combinations of materials listed in any one of Tables 4A and 4B.

Embodiment 64 provides the multi-component fragrance composition of any one of Embodiments 58-63, wherein the low volatile fragrance material is chosen from any of the materials or combinations of materials listed in any one of Tables 2A and 2B.

Embodiment 65 provides the multi-component fragrance composition of any one of Embodiments 58-64, wherein the moderate volatile fragrance material is chosen from any of the materials or combinations of materials listed in any one of Tables 3A and 3B.

Embodiment 66 provides the multi-component fragrance composition of Embodiment 17, wherein the fragrance composition comprises;
a fragrance component present in an amount of from about 0.04 wt % to about 30 wt %, relative to the total weight of the composition, and wherein the fragrance component comprises:
   at least one low volatile fragrance material having a vapor pressure less than 0.001 Torr (0.000133 kPa) at 25° C. present in an amount less than 30 wt % relative to the total weight of the fragrance component;
   at least one moderate volatile fragrance material having a vapor pressure in the range of 0.1 Torr to 0.001 Torr (0.0133 kPa to 0.000133 kPa) at 25° C. present in an amount of higher than 45% relative to the total weight of the fragrance component; and
   at least one high volatile fragrance material having a vapor pressure greater than 0.1 Torr (0.0133 kPa) at 25° C. present in an amount less than 25 wt % relative to the total weight of the fragrance component.

Embodiment 67 provides the multi-component fragrance composition of Embodiment 66, wherein the fragrance composition further comprises at least one substantially non-odorous fragrance modulator present in the amount of from about 0.1 wt % to about 20 wt %, relative to the total weight of the composition.

Embodiment 68 provides the multi-component fragrance composition of any one of Embodiments 66 or 67, wherein the at least one moderate volatile fragrance material is present in an amount of from 45 wt % to about 80 wt % relative to the total weight of the fragrance material.

Embodiment 69 provides the multi-component fragrance composition of any one of Embodiments 66-68, wherein the at least one high volatile fragrance material is present in an amount of from about 1 wt % to about 30 wt % relative to the total weight of the fragrance material.

Embodiment 70 provides the multi-component fragrance composition of any one of Embodiments 66-69, wherein the at least one low volatile fragrance material is present in an amount of from about 1 wt % to about 30 wt % relative to the total weight of the fragrance material.

Embodiment 71 provides the multi-component fragrance composition of any one of Embodiments 66-70, wherein the high volatile fragrance material is chosen from any of the materials or combinations of materials listed in any one of Tables 4A and 4B.

Embodiment 72 provides the multi-component fragrance composition of any one of Embodiments 66-71, wherein the low volatile fragrance material is chosen from chosen from any of the materials or combinations of materials listed in any one of Tables 2A and 2B.

Embodiment 73 provides the multi-component fragrance composition of any one of Embodiments 66-72, wherein the moderate volatile fragrance material is chosen from chosen from any of the materials or combinations of materials listed in any one of Tables 3A and 3B.

Embodiment 74 provides a method of using the coating composition of any one of Embodiments 1-17, the method comprising contacting the fragrance composition of any one of Embodiments 18-73 with the coating composition.

Embodiment 75 provides the method of Embodiment 74, wherein the coating composition is applied to a substrate and the fragrance component is applied to the coating composition.

Embodiment 76 provides the method of Embodiment 75, wherein the substrate comprises keratin or a fabric.

Embodiment 77 provides the method of Embodiment 76, wherein the fragrance component is applied to a substrate and the coating composition is applied to the fragrance component.

Embodiment 78 provides the method of Embodiment 77, wherein the substrate comprises keratin or a fabric.

Embodiment 79 provides the coating composition of any one of Embodiments 1-17, wherein the coating composition is free of a fragrance material.

Embodiment 80 provides a method to enhance the fragrance profile of a composition or improve the longevity of an aroma, comprising bringing into contact or mixing at least one non-odorous fragrance modulator with at least one low volatile fragrance material, high volatile fragrance material, and moderate volatile fragrance material according to a coating composition of any one of Embodiments 18-79.

What is claimed is:

1. A coating composition comprising:
a carrier;
a substantially non-odorous fragrance modulator present in the amount of from about 0.1 wt % to about 50 wt %, relative to the total weight of the composition; and
a fragrance material in a range of from about 0.01 wt % to about 50 wt %.

2. The coating composition of claim 1, where the substantially non-odorous fragrance modulator is present in the amount of from about 5 wt % to about 20 wt %1.

3. The coating composition of claim 1, wherein the at least one substantially non-odorous fragrance modulator is chosen from glucam, methyl glucoside polyol, ethyl glucoside polyol, propyl glucoside polyol, polypropylene glycol-10 methyl glucose ether, ethoxylated methyl glucose ether, polypropylene glycol-20 methyl glucose ether, caprylyl, capryl glucoside, undecyl glucoside, a cyclodextrine, an acrylate, a carboxylated acrylate/octyacrylamide copolymer, or mixtures thereof.

4. The coating composition of claim 1, wherein the at least one substantially non-odorous fragrance modulator is chosen from:
a compound of formula (I): 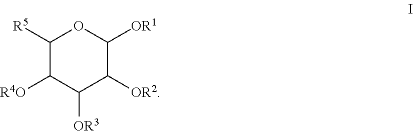
wherein:
R¹ is hydrogen, alkyl, alkenyl or alkynyl;
R² is selected from hydrogen, (C₂-C₂₀)alkyl, (C₂-C₂₀)alkenyl, (C₂-C₂₀)alkynyl, —[R⁶R⁷(R⁸)0]_{w}R⁹, wherein w is from 1 to 10, preferably 2 to 9;
R³ is selected from hydrogen, alkyl, alkenyl, alkynyl, —[R⁶R\R⁸)0]yR⁹, wherein y is
from 1 to 10 or 2 to 9;
R⁴ is selected from hydrogen, alkyl, alkenyl, alkynyl, —[R⁶R\R⁸)0]xR⁹, wherein x is from 1 to 10, preferably 2 to 9;
R⁵ is selected from hydrogen, alkyl, alkenyl, alkynyl, —R⁶0 R⁹, —R⁶0 [R⁶R⁷(R⁸)0]zR⁹,
wherein z is from 1 to 10, preferably 2 to 9;
each R⁶ and R⁷ are independently selected from (C₂-C₂₀)alkylene, (C₂-C₂₀)alkenylene, or (C₂-C₂₀)alkynylene; and
each R⁸ and R⁹ is independently selected from hydrogen or alkyl,
a compound of formula (II): 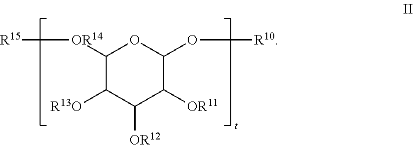
wherein:
R¹⁰ is hydrogen, (C₂-C₂₀)alkyl, (C₂-C₂₀)alkenyl or (C₂-C₂₀)alkynyl;
each R¹¹ is independently selected from hydrogen, (C₂-C₂₀)alkyl, (C₂-C₂₀)alkenyl, (C₂-C₂₀)alkynyl;
each R¹² is independently selected from hydrogen, (C₂-C₂₀)alkyl, (C₂-C₂₀)alkenyl, or (C₂-C₂₀)alkynyl;
each R¹³ is independently selected from hydrogen, (C₂-C₂₀)alkyl, (C₂-C₂₀)alkenyl, or (C₂-C₂₀)alkynyl;
each R¹⁴ is selected from (C₂-C₂₀)alkylene, (C₂-C₂₀)alkenylene, or (C₂-C₂₀)alkynylene; and
R¹⁵ is hydrogen, (C₂-C₂₀)alkyl, (C₂-C₂₀)alkenyl or (C₂-C₂₀) alkynyl;
wherein tis 5 or less, preferably 1, 2 or 3;
Sucrose Laurate, Sucrose Dilaurate, Sucrose Myristate, Sucrose Palmitate, Sucrose Stearate, Sucrose Distearate, Sucrose Tristearate, and their mixtures;
Trimethylcyclohexane derivatives having the formula (III): 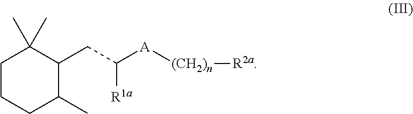
wherein:
n is 0, 1 or 2;
A is C═O or CH—OH;
R^{1a} is hydrogen or methyl;
R^{2a} is a C₂-C₁₀ hydrocarbon group; and
is a saturated or unsaturated carbon-carbon bond;
L-menthoxy ether derivatives having the formula (IV): 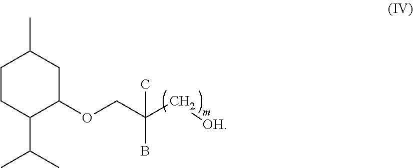
wherein:
m is 0, 1 or 2;
B is hydrogen or OH; and
C is hydrogen or methyl;
Tetra-hydronaphthalene derivatives having the formula (V): 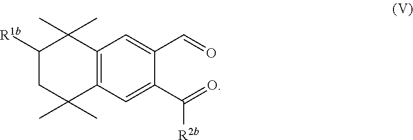
wherein:
R^{1b} is hydrogen or methyl; and
R^{2b} is alkyl;
140
Hyaluronic acid disaccharide sodium salt, sodium hyaluronate and their mixtures;
Ether derivatives having the formula (VI) or formula (VII):
C₅HₗOₘ—(OR^{1c})ₙ (VI).
wherein:
C₅HₗOₘ is a pentose residue, wherein l is an integer from 6 to 9, and m is an integer from 1 to 4;
n is an integer from 1 to 4; and
R^{1c} is C₄-C₂₀ hydrocarbon group; and
C₅H_{c}O_{d}—(OCH₂CH₂—O—CH₂CH₂—O—R^{1c})ₑ (VII).
wherein:
C₆HₓO_{y} is a hexose residue, wherein x is an integer from 7 to 11, and y is an integer from 1 to 5;
z is an integer from 1 to 5; and
R^{1d} is C₄-C₂₀ hydrocarbon group; and
Diethylene Glycol Ether derivatives having the formula (VII) or formula (IX): 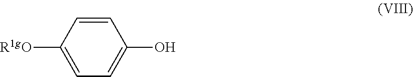
wherein:
C₅H_{c}O_{d} is a pentose residue, wherein c is an integer from 6 to 8, and d is an integer from 1 to 3;
e is an integer from 2 to 4; and
R^{1e} is C₁-C₆ alkyl group; and 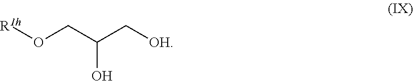
wherein:
C₆H_{f}O_{g} is a hexose residue, wherein f is an integer from 7 to 10, and g is an integer from 1 to 4;
h is an integer from 2 to 5; and
R^{1f} is C₁-C₆ alkyl group;
Hydroquinone Glycoside derivatives having the formula (X):
R¹ⁱOCOR²ⁱCOOR³ⁱ (X).
wherein:
R^{1g} is selected from the group consisting of: (i) pentose residue, hexose residue, aminosaccharide residue, uronic acid residue and their mixtures; (ii) methylated versions of group (i); and (iii) mixtures of groups (i) and (ii); and Propylene Glycol Propyl Ether; Dicetyl Ether; Polyglycerin-4 Ethers; Isoceteth-5; Isoceteth-7, Isoceteth-10; Isoceteth-12; Isoceteth-15; Isoceteth-20; Isoceteth-25; Isoceteth-30; Disodium Lauroamphodipropionate; Hexaethylene glycol monododecyl ether; and their mixtures;
Glyceryl Ether derivatives having the formula (XI): 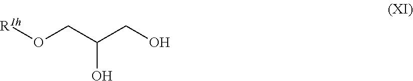
wherein:
R^{1h} is C₄-C₁₂ aliphatic hydrocarbon group;
Panthenol Ethyl Ether, DL-Panthenol and their mixtures;
Aliphatic Dibasic Acid Diester derivatives having the formula (XII):
R¹ⁱOCO^{R2i}COOR³ⁱ (XII).
wherein:
R¹ⁱ is C₄-C₅ alkyl;
R²ⁱ is C₄ alkylene; and
R³ⁱ is C₄-C₅ alkyl; and
Aliphatic Ether derivatives having the formula (XIII):
R⁴ⁱ—O—(CH(CH₃)—CH₂O)ₐ—(CH₂—CH₂)_{b}—H (XIII).
wherein:
a and b are integers such that the sum of a and b is from 1 to 4; and
R⁴ⁱ is an aliphatic chain comprising from 8 to 18 carbons;
N-hexadecyl n-nonanoate, Noctadecyl n-nonanoate and their mixtures;
Tricyclodecane Amide derivatives selected from the group consisting of:
the compounds of formula (XIV): 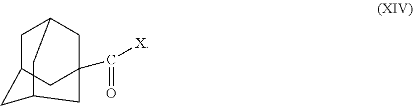
wherein:
X is selected from: 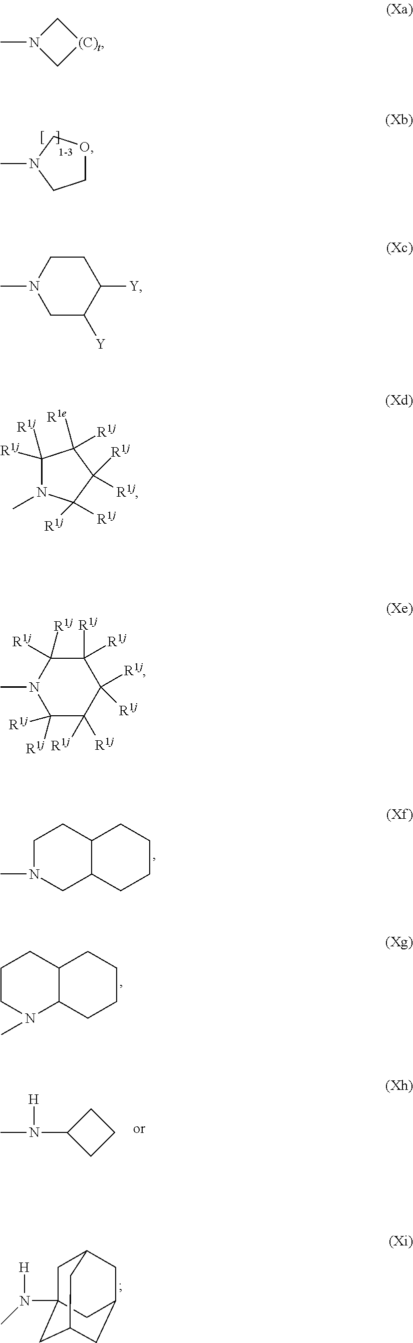
t is 1 to 8;
Y is hydrogen, or a halogen; and
each R^{1j} is independently selected from a hydrogen, or C₁-C₄ alkyl;
the compounds of formula (XV): 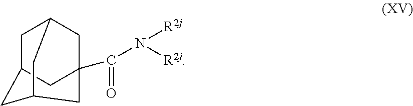
wherein:
each R^{2j} is independently selected from a hydrogen, methyl, ethyl or C₃-C₁₈ alkyl, cycloalkyl or cycloheteroalkyl, with the proviso that both R^{2e} groups are not hydrogen; and
mixtures of the compounds of formulae (XII) and (XIII); and
mixtures thereof.

5. The coating composition of claim 1, wherein the organic carrier is ethanol, dipropylene glycol, benzyl benzoate, diethyl phthalate, isopropyl myristate, (C₁-C₂₀)alkyl, (C₁-C₂₀)alkenyl, (C₁-C₂₀)alkynyl, (C₁-C₂₀)hydroxyl, or mixtures thereof.

6. The coating composition of claim 1, further comprising; a chelator, a thickener, an antioxidant, an emulsifier, or a combination thereof.

7. The coating composition of claim 1, further comprising at least one capsule dispersed in the composition.

8. The coating composition of claim 1, wherein the at least one capsule comprises a fragrance component located therein.

9. The coating composition of claim 7, wherein the at least one capsule comprises a capsule wall comprising polyacrylates; polyethylenes, polyamides; polystyrenes, polyisoprenes, polycarbonates; polyesters; polyureas; polyurethanes, polyolefins; polysaccharides; epoxy resins; vinyl polymers; urea cross-linked with formaldehyde or gluteraldehyde; melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization; silk; wool; gelatine; cellulose; proteins; and combinations thereof.

10. The coating composition of claim 7, wherein the at least one capsule is configured to degrade.

11. The coating composition of claim 1, wherein the fragrance material is present in a range of from about 0.1 wt % to about 25 wt %.

12. The coating composition of claim 1, wherein the coating composition is a primer or a top-coat.

13. The coating composition of claim 1, wherein the fragrance material comprises:
at least one low volatile fragrance material having a vapor pressure less than 0.001 Torr (0.000133 kPa) at 25° C.;
at least one moderate volatile fragrance material having a vapor pressure in the range of 0.1 Torr to 0.001 Torr (0.0133 kPa to 0.000133 kPa) at 25° C.;
at least one high volatile fragrance material having a vapor pressure greater than 0.1 Torr (0.0133 kPa) at 25° C.; or
mixtures thereof.

14. A multi-component fragrance composition comprising:
the coating composition of claim 1;
a fragrance composition in contact with the coating composition.

15. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C. present in an amount greater than 30 wt %, relative to the total weight of the fragrance component;
at least one moderate volatile fragrance material having a vapor pressure in the range of 0.1 Torr to 0.001 Torr (0.0133 kPa to 0.000133 kPa) at 25° C. present in an amount of from about 30 wt % to about 70 wt %, relative to the total weight of the fragrance component; and
at least one high volatile fragrance material having a vapor pressure greater than 0.1 Torr (0.0133 kPa) at 25° C. present in an amount of from about 0 wt % to about 30 wt % relative to the total weight of the fragrance component.

16. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C. present in an amount of from about 0 wt % to about 30 wt % relative to the total weight of the fragrance component;
at least one moderate volatile fragrance material having a vapor pressure in the range of 0.1 Torr to 0.001 Torr (0.0133 kPa to 0.000133 kPa) at 25° C. present in an amount of from about 30 wt % to about 70 wt %, relative to the total weight of the fragrance component;
at least one high volatile fragrance material having a vapor pressure greater than 0.1 Torr (0.0133 kPa) at 25° C. present in an amount greater than about 30 wt % relative to the total weight of the fragrance component or;
a mixture thereof.

17. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C. present in an amount or from about 30 wt % to about 70 wt %, relative to the total weight of the fragrance component;
at least one moderate volatile fragrance material having a vapor pressure in the range of 0.1 Torr to 0.001 Torr (0.0133 kPa to 0.000133 kPa) at 25° C. present in an amount less than about 30 wt %, relative to the total weight of the fragrance component; and
at least one high volatile fragrance material having a vapor pressure greater than 0.1 Torr (0.0133 kPa) at 25° C. present in an amount of from about 0.1 wt % to about 30 wt % relative to the total weight of the fragrance component.

18. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C.;
at least one moderate volatile fragrance material having a vapor pressure in the range of 0.1 Torr to 0.001 Torr (0.0133 kPa to 0.000133 kPa) at 25° C.; and
at least one high volatile fragrance material having a vapor pressure greater than 0.1 Torr (0.0133 kPa) at 25° C.,
wherein the at least one low volatile fragrance material, at least one moderate volatile fragrance material, and at least one high volatile fragrance material are individually present in substantially the same amount relative to the total weight of the fragrance component.

19. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C. present in an amount of greater than about 30 wt % relative to the total weight of the fragrance component;
at least one high volatile fragrance material having a vapor pressure greater than 0.1 Torr (0.0133 kPa) at 25° C. present in an amount greater than about 30 wt % relative to the total weight of the fragrance component; and
at least one moderate volatile fragrance material having a vapor pressure in the range of 0.1 Torr to 0.001 Torr (0.0133 kPa to 0.000133 kPa) at 25° C. present in an amount of less 30 wt %, relative to the total weight of the fragrance component.

20. The multi-component fragrance composition of claim 14, wherein the fragrance composition comprises:
a fragrance component present in an amount of from about 0.04 wt % to about 30 wt %, relative to the total weight of the composition, and wherein the fragrance component comprises at least one of:
at least one low volatile fragrance material having a vapor pressure less than 0.001 Torr (0.000133 kPa) at 25° C. present in an amount of from about 30 wt % to about 40 wt %, relative to the total weight of the fragrance component;
at least one moderate volatile fragrance material having a vapor pressure in the range of 0.1 Torr to 0.001 Torr (0.0133 kPa to 0.000133 kPa) at 25° C. present in an amount of from about 30 wt % to about 40 wt %, relative to the total weight of the fragrance component; and
at least one high volatile fragrance material having a vapor pressure greater than 0.1 Torr (0.0133 kPa) at 25° C. present in an amount of from about 30 wt % to about 40 wt %, relative to the total weight of the fragrance component.
